SEQUENCE LISTING (24 sequences)

SEQ ID NO: 1 - DNA, 1946 bp, Mus musculus
acagtgtgggagatggcggaaccactgaggggacgtggtccgaggtcccgcggtggccgaggcgctcggagagcccgaggcgcccgtggccggtgtcctcgcgcccggcagtctccggctaggctcattccagacaccgtgcttgtggacttggtcagtgacagcgacgaagaggtcttggaagtcgcagacccagtagaggtgccggtcgcccgcctccccgcgccggctaaacctgagcaggacagcgacagtgacagtgaaggggcggccgaggggcctgcgggagccccgcgtacattggtgcgacggcggcggcggcggctgctggatcccggagaggcgccggtggtcccagtgtactccgggaaggtacagagcagcctcaacctcattccagataattcatccctcttgaaactgtgcccttcagagcctgaagatgaggcagatctgacaaattctggcagttctccctctgaggatgatgccctgccttcaggttctccctggagaaagaagctcagaaagaagtgtgagaaagaagaaaagaaaatggaagagtttccggaccaggacatctctcctttgccccaaccttcgtcaaggaacaaaagcagaaagcatacggaggcgctccagaagctaagggaagtgaacaagcgtctccaagatctccgctcctgcctgagccccaagcagcaccagagtccagcccttcagagcacagatgatgaggtggtcctagtggaagggcctgtcttgccacagagctctcgactctttacactcaagatccggtgccgggctgacctagtgagactgcctgtcaggatgtcggagccccttcagaatgtggtggatcacatggccaatcatcttggggtgtctccaaacaggattcttttgctttttggagagagtgaactgtctcctactgccacccctagtaccctaaagcttggagtggctgacatcattgattgtgtggtgctagcaagctcttcagaggccacagagacatcccaggagctccggctccgggtgcaggggaaggagaaacaccagatgttggagatctcactgtctcctgattctcctcttaaggttctcatgtcacactatgaggaagccatgggactctctggacacaagctctccttcttctttgatgggacaaagctttcaggcaaggagctgccagctgatctgggcctggaatccggagatctcatcgaagtctggggctgaagctctcaccctgttcggacgcaaagccaagacatggagacaatagctcccaattttattattgtgatttttcgccccataagggctaacagaaactgaattagaacttgtttacttatttatttctggtgctggggattgaaccccagactatgcacatgctaaggatgtatgaagtggaggcaaaaccaaggcattacctttagccagcctctagtagactgtagtgtcaagcaagtggctacttggtagttgtgtggctctgtgtatgtttgtgctgtatttggcagcccctggggcacatagaagggaccttggcttccctaccatttcacgttcgctggtgccctttccttcatcagatgacttctgtgaagctgcctatgttgagtgtgttgaactaaatgagctctgctttgggtgtccaggcctggggtttgtgccgcagttggagccagcagtgacttcactctgacttgggactgagaatgcatttcctggtggagacactcgggtgcagaaatataacagaaggtgacatacatgctgaagctgaggactaggtcgaaagttaacgacgttgcattttcagccttgggtatcctctctgcctgccaggactctagccagtgtctggtacacacttcttggcatggacacctaggtcgacgcgggcgcgattcggccgactcgag

SEQ ID NO: 2 - protein, 412 aa, Mus musculus
MAEPLRGRGPRSRGGRGARRARGARGRCPRARQSPARLIPDTVLVDLVSDSDEEVLEVADPVEVPVARLPAPAKPEQDSDSDSEGAAEGPAGAPRTLVRRRRRRLLDPGEAPVVPVYSGKVQSSLNLIPDNSSLLKLCPSEPEDEADLTNSGSSPSEDDALPSGSPWRKKLRKKCEKEEKKMEEFPDQDISPLPQPSSRNKSRKHTEALQKLREVNKRLQDLRSCLSPKQHQSPALQSTDDEVVLVEGPVLPQSSRLFTLKIRCRADLVRLPVRMSEPLQNVVDHMANHLGVSPNRILLLFGESELSPTATPSTLKLGVADIIDCVVLASSSEATETSQELRLRVQGKEKHQMLEISLSPDSPLKVLMSHYEEAMGLSGHKLSFFFDGTKLSGKELPADLGLESGDLIEVWG

SEQ ID NO: 3 - DNA, 3469 bp, Mus musculus
atgccaaataccagctttccagtcccttccaagtttccactcggccctccggccgcagtctgcgggagcggagaaactttgcggcccgcgccgccctccggcggcaccatgaaggcggccgaggaagaacactacagttatgtgtcccctagtgtcacctcgaccctgccccttcccacagcacactctgccttgccagcagcatgccacgacctccagacgtccaccccgggtatctcagctgttccttcagccaatcatccccccagttacggaggggctgtggacagcgggccttcgggatacttcctgtcctctggcaacaccagacccaacggggccccgactctggagagtccgagaatcgagatcacctcctacctgggcctacaccatggcagcggccagtttttccacgacgtggaggtggaagacgtacttcctagctgcaagcgctcaccgtctacagcaaccctgcacctgcccagcctggaagcctacagagacccctcctgcctgagcccagccagcagtctctcctccagaagctgtaactctgaggcctcctcctacgagtccaactactcctacccatacgcgtccccccagacctctccgtggcagtcaccctgcgtgtctcccaagaccacggacccggaggagggttttccccgaagcctgggtgcctgccacctgctaggatcgcccaggcactccccatccacctctcctcgggcaagcatcacggaggagagctggctcggtgcccgcggctcccggcccacgtccccctgcaacaagcgcaagtacagtctcaatggccggcagccctcctgctcaccccaccactcacccacaccatccccccatggctcccctcgggtcagtgtgaccgaagatacctggctcggtaacaccacccagtataccagctctgccattgtggcagccatcaacgccctgaccaccgatagcactctggacctgggtgatggggtccctatcaagtctcgaaagacagcactggagcatgcgccctctgtggccctcaaagtagagccagctggggaagacctgggcaccactccacccacttctgacttcccacccgaggagtacaccttccagcaccttcggaagggtgccttttgcgagcagtatctgtcggtgccacaggcctcgtatcagtgggcgaagcccaagtctctttccccgacatcatatatgagcccatccttgcctgcccttgactggcagctcccgtcacattctggtccatacgagcttcggatcgaggtgcagcccaagtctcaccacagggctcactatgagacggaaggcagccggggggctgtgaaggcttcagctggaggacaccccattgtgcagctacacggttacttggagaatgaacctctcacgctacagctgttcattgggacggctgacgaccgcctgctgaggccccacgccttctaccaggtccaccggatcacggggaagactgtctccaccaccagccacgagatcatcctgtccaacaccaaagtcctggagatcccgttgcttccagaaaataacatgcgagccatcatcgactgtgctgggatcctgaagctcagaaactctgatattgagctgaggaaaggggagacagacatcgggaggaagaacaccagggtgaggctggtcttccgagttcacatcccacagcccaatggccggacgctgtctctccaggtggcctcgaaccctatcgagtgttcccagcggtcagcccaggagctgcccctcgtggagaagcagagcacagacagctacccagtcatcggcgggaagaagatggtgctgtctggccataactttctgcaagactccaaagtcattttcgtggagaaggctccagatggccaccacgtctgggagatggaagcaaagactgaccgggacctgtgcaagccaaattccctggtggttgagataccacctttccgcaaccagaggataaccagccccgcccaagtcagtttctatgtctgcaacgggaaacggaagagaagccagtaccagcgtttcacgtaccttcctgccaatggtaactctgtctttctaaccttaagctctgagagtgagctgagaggaggtttttactgagcagccccccgaggctataagaggatgttgttgtaaacaaaacaaaacaaaacaaaacatacctgtagcctcttcacaccacgtgatagccctattcacaagaccaagtcgcccaccccctcaaagaaaagcgaagcctgggtgtgttttcctgtgactggtgcatgctggggtcatcacttgctcgccttttgcaaatacagcagcgcggccaaccaagcagctctgctgcgctcaggggctgatgcggtctgggggtgtatatctaacctctgtgagtctttgggttagaagaaagtatttgtcaacgcagttttgtaagtagcttcgaaaataagcctgccgtggtcactggggaacatacatgatgttgtcctcatggtgacgcttctacacagcgtgcggtgtgtctccactgaataatgctgtcccctggtgacgtgagactttcagatggaagctcttctgctcgagtttactcatttagggaatggcttctttcattcagaagtgatcggctcgccttttcaactttctagggtgttttatttacgaaaataccgtttttaactgctccccgccccgcaagcttctagaaaggtgtgtcccaggcgtccagggtttcctgtgtggtgcaggccattctcctgcagcaggatgtataaacagagagcagagtcggttgttatcctgagttctattgtattttgagtaagctaggctatgtcaacaacctttttaaattgctacttttttttttcctctaaaaacttaagatagtcatgtaatttaagagggaagttatacaataaatactagccatgaaagcagccatattgctatcttagtaaaatcaaggtggttttgttgttgttattttgttttgttttttgttttttaaggtttcaaggttttttgtttttgaagtgtaaaggcatttggaacagtttagacagtacgaaaagttggtattaaaattctgaaaccaattgtcttatcaggaaacccctagaaatgccctttaaaaatgaggacaatagctttgttgcattctcaaacaaggacatcagtgaaagggcagcaactgtctgtgctgtgggtgaccccagaacagcggcccatcccccatcccgtctctgctcttcagattatttcacaggcctcttcctttcgggaaataatgcacactctctcttacaaaaaaaccaaacatttggtcttttattttattttattttattttttgaaagtgcaatgattgtgtcctacctatacttcaagcatggttgatctaagatttttgaaaggtctaaac

SEQ ID NO: 4 - protein, 717 aa, Mus musculus
MPNTSFPVPSKFPLGPPAAVCGSGETLRPAPPSGGTMKAAEEEHYSYVSPSVTSTLPLPTAHSALPAACHDLQTSTPGISAVPSANHPPSYGGAVDSGPSGYFLSSGNTRPNGAPTLESPRIEITSYLGLHHGSGQFFHDVEVEDVLPSCKRSPSTATLHLPSLEAYRDPSCLSPASSLSSRSCNSEASSYESNYSYPYASPQTSPWQSPCVSPKTTDPEEGFPRSLGACHLLGSPRHSPSTSPRASITEESWLGARGSRPTSPCNKRKYSLNGRQPSCSPHHSPTPSPHGSPRVSVTEDTWLGNTTQYTSSAIVAAINALTTDSTLDLGDGVPIKSRKTALEHAPSVALKVEPAGEDLGTTPPTSDFPPEEYTFQHLRKGAFCEQYLSVPQASYQWAKPKSLSPTSYMSPSLPALDWQLPSHSGPYELRIEVQPKSHHRAHYETEGSRGAVKASAGGHPIVQLHGYLENEPLTLQLFIGTADDRLLRPHAFYQVHRITGKTVSTTSHEIILSNTKVLEIPLLPENNMRAIIDCAGILKLRNSDIELRKGETDIGRKNTRVRLVFRVHIPQPNGRTLSLQVASNPIECSQRSAQELPLVEKQSTDSYPVIGGKKMVLSGHNFLQDSKVIFVEKAPDGHHVWEMEAKTDRDLCKPNSLVVEIPPFRNQRITSPAQVSFYVCNGKRKRSQYQRFTYLPANGNSVFLTLSSESELRGGFY

SEQ ID NO: 5 - DNA, 3419 bp, Mus musculus
cacagatacggtgacccctgctctgcgccccgcgactctatacgaacccgcatcccgaacgccccgagccatggacgtcccggagccgcagcccgaccccgatggcggggacggccccggccacgagcccgggggcagtccccaagacgagctggacttttccatcctcttcgattatgactatctgaaccctatcgaagaagaaccgatcgcacataaggccatcagctcaccctccggactcgcatacccggatgatgtcctggactatggcctcaagccatgcaacccccttgccagtctctctggcgagccccctggccggttcggagagccggatagtatagggttccagaactttctgagcccggtcaagccagcaggggcttcgggcccgagccctcggatcgagatcactccatcccacgaactgatgcaggcagggggggccctccgtgggagagacgccggcctgtcccccgagcagccggccctggccctggccggcgtggccgccagcccgaggttcacactgcccgtgcccggctacgagggctaccgcgagccgctttgcttgagccccgctagcagcggctcctctgccagcttcatttctgacaccttctccccctacacctcgccctgcgtctcacccaataacgccgggcccgacgacctgtgtccccagtttcaaaacatccctgctcattattcccccagaacctctccaataatgtcacctcgaaccagcctcgccgaggacagctgcctgggccgacactcgcccgtgccccgtccggcatcccgctcctcctcacccggtgccaagcggaggcattcgtgcgcagaggctttggttgctcctctgcccgcagcctcaccccagcgctcccggagcccctcgccacagccctcgcctcacgtggcaccgcaggacgacagcatccccgctgggtacccccccacggccggctctgctgttctcatggatgccctcaacaccctggccaccgactcgccctgcgggatcccctccaagatatggaagaccagtcctgacccgacgcctgtgtccaccgctccgtccaaggctggcctggcccgccacatctaccctactgtggagttcctggggccatgtgagcaggaggagaggaggaattccgctccagagtccatcctgctggtaccacctacttggcccaagcagttggtgccggccattcccatctgcagcatccctgtgactgcatccctcccaccactcgagtggccactctccaatcagtcgggctcctatgagctacggattgaggtccaacccaagccccatcaccgggcccactatgagacggagggcagccgtggcgctgtcaaagccccaacaggaggacaccctgtggtgcagctccacggctacatggagaacaagcctctggggcttcagatcttcattgggacagcagatgagaggatccttaagccgcacgccttctaccaagtacacaggatcactgggaaaacggtcaccaccacgagctatgagaagatcgtaggcaacaccaaggtcctggagatccccctggagccaaagaacaacatgagagccaccatcgactgtgcaggcatcctgaagctccgaaacgctgacatcgagctgcggaagggcgagacggacatcggcaggaagaacacgcgtgtgcgcctggtgttccgcgtgcacgtcccagagcccagtgggcgcatcgtctccctgcaggctgcgtccaaccccatcgagtgctctcagcgctctgcccacgagctgcccatggtggagagacaagacatggacagctgcctggtctacgggggccagcagatgatcctcacgggccagaacttcacagcggagtccaaggttgtgttcatggagaagactacagatgggcagcagatttgggagatggaagctacggtggataaagacaagagccagcctaacatgctttttgttgagatccccgagtatcggaacaagcacatccgcgtgcccgtgaaagtcaacttctacgtcatcaacggaaagaggaaacgaagtcagccacagcactttacctaccacccagtccctgccatcaagacagagcccagcgatgagtatgaaccatctttgatctgcagccccgcccatggaggcctggggagccagccatattacccacagcacccaatgctggccgagtccccctcctgccttgtggctaccatggccccctgccaacagttccgctcggggctctcatcccccgatgctcgctaccaacagcagagccccgcagctgccctctaccagagaagcaagagcctgagtcccggcctgctgggctaccagcagccgtccctcctggcagcacccttgggtctggctgatgcccaccgctctgtgctggtgcatgctggttctcaggggcaggggcagggctccaccctccgacacacatcctcggccagccagcaggcctcacccgtgatccactactcacccaccaaccagcagcttcgcggtgggggtcaccaggagttccagcatatcatgtactgtgaaaacttcggccccagctctgccaggcctggcccgcctcccatcaaccaaggtcagaggctgagcccgggcgcctaccccacagtcatccaacaacagactgccccgagccaaagagctgccaaaaacggacccagtgaccagaaggaagctctgcccacgggagtgaccgtcaaacaggaacagaacctggaccagacctacctggatgacgcagccacttcagaaagctgggttgggacagaaaggtatatagagagaaaattttggaagaagacccttgtgcagcctgggctcctgccctcatttttacttcttggctccctgtctgctggaccaaggtcacagacaccatcagaaagaaagcccatagaggaagacgtgcccttgagttgcagccagatagcctggtgttgtcagcatcccttggggacctgccctgtcctgccagggcctttagctgtagagtggtgggaagggcagctcgggcgtgggctggagccaattccctgggctccagacagtgccggcagcctccatgaggtggacagtgtaggcctggcgggagtggtcggaatggttctgctcactcttatgcaccacttctccatggatcagaaccagaccccctctcctcactggcaaaggcacaaagaggttgctagcccaggctggatctgacccgaggaagctggtgccaggcccagagtctgaaggggctcgaatcatccttcttgacaccccacgggtatgggagccagggatgaaccagaggcaaccgttctccagcatggcatcctccatcgcaatccacaggcccaacactcggcctagg

SEQ ID NO: 6 - protein, 1064 aa, Mus musculus
MDVPEPQPDPDGGDGPGHEPGGSPQDELDFSILFDYDYLNPIEEEPIAHKAISSPSGLAYPDDVLDYGLKPCNPLASLSGEPPGRFGEPDSIGFQNFLSPVKPAGASGPSPRIEITPSHELMQAGGALRGRDAGLSPEQPALALAGVAASPRFTLPVPGYEGYREPLCLSPASSGSSASFISDTFSPYTSPCVSPNNAGPDDLCPQFQNIPAHYSPRTSPIMSPRTSLAEDSCLGRHSPVPRPASRSSSPGAKRRHSCAEALVAPLPAASPQRSRSPSPQPSPHVAPQDDSIPAGYPPTAGSAVLMDALNTLATDSPCGIPSKIWKTSPDPTPVSTAPSKAGLARHIYPTVEFLGPCEQEERRNSAPESILLVPPTWPKQLVPAIPICSIPVTASLPPLEWPLSNQSGSYELRIEVQPKPHHRAHYETEGSRGAVKAPTGGHPVVQLHGYMENKPLGLQIFIGTADERILKPHAFYQVHRITGKTVTTTSYEKIVGNTKVLEIPLEPKNNMRATIDCAGILKLRNADIELRKGETDIGRKNTRVRLVFRVHVPEPSGRIVSLQAASNPIECSQRSAHELPMVERQDMDSCLVYGGQQMILTGQNFTAESKVVFMEKTTDGQQIWEMEATVDKDKSQPNMLFVEIPEYRNKHIRVPVKVNFYVINGKRKRSQPQHFTYHPVPAIKTEPSDEYEPSLICSPAHGGLGSQPYYPQHPMLAESPSCLVATMAPCQQFRSGLSSPDARYQQQSPAAALYQRSKSLSPGLLGYQQPSLLAAPLGLADAHRSVLVHAGSQGQGQGSTLRHTSSASQQASPVIHYSPTNQQLRGGGHQEFQHIMYCENFGPSSARPGPPPINQGQRLSPGAYPTVIQQQTAPSQRAAKNGPSDQKEALPTGVTVKQEQNLDQTYLDDAATSESWVGTERYIERKFWKKTLVQPGLLPSFLLLGSLSAGPRSQTPSERKPIEEDVPLSCSQIAWCCQHPLGTCPVLPGPLAVEWWEGQLGRGLEPIPWAPDSAGSLHEVDSVGLAGVVGMVLLTLMHHFSMDQNQTPSPHWQRHKEVASPGWI

SEQ ID NO: 7 - DNA, 3638 bp, Mus musculus
cacgccgatgactactgcaaactgtggcgcccacgacgagctcgacttcaaactcgtctttggcgaggacggggcgccggcgccgccgcccccgggctcgcggcctgcagatcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtatccaatttacatctatctctcctaactgtcaacaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgctgcacgattcactcttggttcacctctgacttctccaggtggctctccaggaggttgccctggagaagagtcctggcatcaacagtatggatctggacactccttgtcacctaggcaatctccttgccactctcctagatctagtatcactgatgagaattggctgagccccagaccagcctcaggaccctcatcaaggcccacttctccctgtggtaaacgacggcactccagtgctgaagtatgttatgctggttccctttcaccccatcactcgcctgttccatcccctggtcactctcctagaggaagtgtaacagaagatacctggctcactgctcctgtccacactggatcaggcctcagccctgcaccgtttccatttcagtactgtgtagagactgacatccctttgaaaacaaggaagacttctgaagatcaagctgccatactaccaggaaaattagagatctgttcagatgatcaagggaacttatccccttcccgggagacatcagtagatgatggccttggatctcagtatcctttaaagaaagattcatctggtgaccaatttctttcagttccttcaccctttacctggagcaaaccaaagcctggccacacccctatatttcgcacatcttcattacctccattagactggcctttaccaactcactttggacaatgtgaattgaaaatagaagtgcaacctaaaactcaccatagagcccattatgaaactgaaggtagccgaggggcagtgaaagcctctactggtggccatcctgttgtgaagctcctgggctatagtgaaaagccaataaatctacagatgtttattggaacagccgatgatagatatttacgacctcatgcattttaccaggtgcatcggattactggaaagacagttgctactgcaagtcaagagataataatcgccagcacaaaagttctggaaatcccacttctacctgaaaataatatgtcagcgagtattgactgtgcaggtattttaaagctccgcaactcagatatagaacttcgaaaaggagaaactgatattggcagaaaaaatactagagtccgacttgtatttcgtgtgcacatcccacagcccagtggaaaagttctttctctacagatagcatctattcctgttgagtgctctcagcgatctgctcaagaactccctcatattgagaagtacagtatcaacagttgctctgtcaatggaggccacgaaatgattgtgactggatctaattttcttccagaatccaaaataatttttcttgaaaaaggacaagatggaggacctcattgggaggttgaaggaaaaataatcagggaaaaatgtcaaggggctcacattgtccttgaagttcctccctatcataacccagcagttacatctgccgtgcaggtgcacttttatctttgcaatggcaagaggaaaaaaagccagtctcaacgttttacttacacaccagttttgatgaagcaagaacaaagagaagacacagatttgccttcagttccatctttgcctgtgcctcattctgcccaggcccagaggccctcctcagagacagggcacccccatgaccgtgcgatgtcagcaccgggaggcttgctttgtcaagtgcagccagcatatacatctatggtagcctccacccatttgccacagttgcagtgtagggatgaaggtgctggtaaagagcagcacatagcaacatcttcagtcatgcaccagcctttccaagttacaccaacatctcctatagggtcttcctatcagtctatacaaactagtatgtataacggtccaacctgtcttcctgtgaatcctgcctctagtcaagaatttgacccagttttgtttcagcaagatgcagctctttctagtttagtaaatcttggctgtcaaccactgtcaccaataccatttcattcttcaaattcagatgcgacaggacatctcttagcacattcacctcattctgtgcaaaccccacctcatctgcagtcaatgggataccattgttcaaatgcaggacaaacagctctttcttctccagtggcagaccagatcacaggtcagccttcttctcatttacagcctattacatattgtccttcacacccaggatctgctacagcagcttccccagcagcctctcatcctctggctagttcaccgatttctgggccatcatcccctcagctgcagcctatgccttatcaatctcctagctcaggaactgcctcatcaccatctccaactaccagaatgcattctggacagcactcaactcaagcacaaagtacaggccaaggaggcctttctgttccttcatccttagtatgtcacagtttgtgtgacccagcgtcatttccgcctggtggtgcaactgtgagcattaaacctgaacctgaagatcaagaacctaactttgcaaccattggtctacaggatatcaccttagatgatgtgaacgagattattgggagagacatgtcccagatttctgtttcccaagccacggaggtgatgagagacactcctctcccgggtccggcgtctccagatctgatgacctctcacagtgctcactgagccttttgcccaccacggactgctcagcctgcttctctcccggaacttaaggtccagccagacttcaggatctgttgcaggagttggactcactgtgggaaaagcagcgttccctctccttaagccacaggcagattgtgtaaaacaggaacagcagcggctgtttcagctgtgggaaacgagctttgcttttatatatatatatatatatatatatatatatttaaataggatacttttatatgatgggtgctttgagtgtgaatgcagcaggctctatctcagaggtgctgctcttgcaggtgacctggttgcatagctaggattagtaatttgtactgctttctggtcatttgaagagccctttagttttgatgatatttttaaaatagaactttttgat

SEQ ID NO: 8 - protein, 1075 aa, Mus musculus
MTTANCGAHDELDFKLVFGEDGAPAPPPPGSRPADLEPDDCASIYIFNVDPPPSTLTTPLCLPHHGLPSHSSVLSPSFQLQSHKNYEGTCEIPESKYSPLGGPKPFECPSIQFTSISPNCQQELDAHEDDLQINDPEREFLERPSRDHLYLPLEPSYRESSLSPSPASSISSRSWFSDASSCESLSHIYDDVDSELNEAAARFTLGSPLTSPGGSPGGCPGEESWHQQYGSGHSLSPRQSPCHSPRSSITDENWLSPRPASGPSSRPTSPCGKRRHSSAEVCYAGSLSPHHSPVPSPGHSPRGSVTEDTWLTAPVHTGSGLSPAPFPFQYCVETDIPLKTRKTSEDQAAILPGKLEICSDDQGNLSPSRETSVDDGLGSQYPLKKDSSGDQFLSVPSPFTWSKPKPGHTPIFRTSSLPPLDWPLPTHFGQCELKIEVQPKTHHRAHYETEGSRGAVKASTGGHPVVKLLGYSEKPINLQMFIGTADDRYLRPHAFYQVHRITGKTVATASQEIIIASTKVLEIPLLPENNMSASIDCAGILKLRNSDIELRKGETDIGRKNTRVRLVFRVHIPQPSGKVLSLQIASIPVECSQRSAQELPHIEKYSINSCSVNGGHEMIVTGSNFLPESKIIFLEKGQDGGPHWEVEGKIIREKCQGAHIVLEVPPYHNPAVTSAVQVHFYLCNGKRKKSQSQRFTYTPVLMKQEQREDTDLPSVPSLPVPHSAQAQRPSSETGHPHDRAMSAPGGLLCQVQPAYTSMVASTHLPQLQCRDEGAGKEQHIATSSVMHQPFQVTPTSPIGSSYQSIQTSMYNGPTCLPVNPASSQEFDPVLFQQDAALSSLVNLGCQPLSPIPFHSSNSDATGHLLAHSPHSVQTPPHLQSMGYHCSNAGQTALSSPVADQITGQPSSHLQPITYCPSHPGSATAASPAASHPLASSPISGPSSPQLQPMPYQSPSSGTASSPSPTTRMHSGQHSTQAQSTGQGGLSVPSSLVCHSLCDPASFPPGGATVSIKPEPEDQEPNFATIGLQDITLDDVNEIIGRDMSQISVSQATEVMRDTPLPGPASPDLMTSHSAH

SEQ ID NO: 9 - DNA, 1282 bp, Mus musculus
ggactcgggtgaagatggcggcagccgaggccgcgaactgcatcatggagaattttgtagccaccttggctaatgggatgagcctccagccgcctcttgaagaagtttcctgtggccaagcagaaagtagtgagaagcccaacgctgaggacatgacatccaaagactactactttgactcctatgcccactttggcatccacgaggagatgctgaaggatgaggtgcgcaccctcacataccgcaactccatgtttcacaatcggcatctcttcaaagacaaggtggtgctggatgtgggctcaggcactggcatcctctgcatgtttgctgccaaggcgggggcccgcaaggttattgggattgagtgttccagtatctccgattatgctgtgaagattgtcaaagccaacaagttagaccatgtggtgaccatcatcaagggcaaggtggaggaggtggagctgcccgtggagaaggtggacatcatcatcagcgagtggatgggttactgcctcttctacgagtccatgctcaacaccgtgctgcacgctcgggacaagtggctggcacccgatggcctcatcttcccagaccgggccaccttgtatgtgacagccattgaggaccgacaatataaagactacaagatccactggtgggagaacgtgtatggctttgatatgtcctgcattaaagacgtggccatcaaggagcccctggtggacgtggtggacccaaagcagctggtcaccaatgcctgcctcataaaggaggtggacatttacacagtcaaggtggaggacctgaccttcacctcccccttctgcctgcaagtgaagaggaacgactacgtgcacgcgctggtggcttacttcaacatcgagttcacccgatgccacaagaggaccggcttctccaccagtcctgagtccccgtacacacactggaagcagactgtgttctacatggaggactacctaacagtgaagactggcgaggagatctttggcaccattggaatgaggcccaatgccaaaaacaatcgtgacttggactttaccatcgacctggacttcaagggtcagctgtgtgagctctcttgttccaccgactaccggatgcgctgaggaggtgccaggctggccctcctgcagaagggggctcggggggatgggcttgggggatgggggggtacatcgtgactgtgtttttcataacttatgtttttatatggttgcgtttatgccaataaatcctcagctgaccatgaaaaaaaaaa

SEQ ID NO: 10 - protein, 371 aa, Mus musculus
MAAAEAANCIMENFVATLANGMSLQPPLEEVSCGQAESSEKPNAEDMTSKDYYFDSYAHFGIHEEMLKDEVRTLTYRNSMFHNRHLFKDKVVLDVGSGTGILCMFAAKAGARKVIGIECSSISDYAVKIVKANKLDHVVTIIKGKVEEVELPVEKVDIIISEWMGYCLFYESMLNTVLHARDKWLAPDGLIFPDRATLYVTAIEDRQYKDYKIHWWENVYGFDMSCIKDVAIKEPLVDVVDPKQLVTNACLIKEVDIYTVKVEDLTFTSPFCLQVKRNDYVHALVAYFNIEFTRCHKRTGFSTSPESPYTHWKQTVFYMEDYLTVKTGEEIFGTIGMRPNAKNNRDLDFTIDLDFKGQLCELSCSTDYRMR

SEQ ID NO: 11 - DNA, 21 bp, Artificial Sequence
  RNA molecule with two deoxythymidines at 3′ end
ccucauuccagauaauucatt

SEQ ID NO: 12 - DNA, 21 bp, Artificial Sequence
  RNA molecule with two deoxythymidines at 3′ end
ugaauuaucuggaaugaggtt

SEQ ID NO: 13 - DNA, 21 bp, Artificial Sequence
  RNA molecule with two deoxythymidines at 3′ end
gugaacaagcgucuccaautt

SEQ ID NO: 14 - DNA, 21 bp, Artificial Sequence
  RNA molecule with two deoxythymidines at 3′ end
cuuggagacgcuuguucactt

SEQ ID NO: 15 - DNA, 21 bp, Artificial Sequence
  RNA molecule with two deoxythymidines at 3′ end
uccggagaucucaucgaagtt

SEQ ID NO: 16 - DNA, 21 bp, Artificial Sequence
  RNA molecule with two deoxythymidines at 3′ end
cuucgaugagaucuccggatt

SEQ ID NO: 17 - DNA, 21 bp, Artificial Sequence
  RNA molecule with two deoxythymidines at 3′ end
agacaagguggugcuggautt

SEQ ID NO: 18 - DNA, 21 bp, Artificial Sequence
  RNA molecule with two deoxythymidines at 3′ end
auccagcaccaccuugucutt

SEQ ID NO: 19 - DNA, 21 bp, Artificial Sequence
  RNA molecule with two deoxythymidines at 3′ end
gguggacaucaucaucagctt

SEQ ID NO: 20 - DNA, 21 bp, Artificial Sequence
  RNA molecule with two deoxythymidines at 3′ end
gcugaugaugauguccacctt

SEQ ID NO: 21 - DNA, 21 bp, Artificial Sequence
  RNA molecule with two deoxythymidines at 3′ end
gacuggcgaggagaucuuutt

SEQ ID NO: 22 - DNA, 21 bp, Artificial Sequence
  RNA molecule with two deoxythymidines at 3′ end
aaagaucuccucgccaguctt

SEQ ID NO: 23 - DNA, 1593 bp, Mus musculus
  CDS (1)..(1590)
atgggcatcgtggagccgggctgcggagacatgctgaccggcaccgagccgatgccgagtgacgagggccgggggcccggagcggaccaacagcatcgtttcttctatcccgagccgggcgcacaggacccgaccgatcgccgcgcaggtagcagcctggggacgccctactctgggggcgccctggtgcctgccgcgccgggtcgcttccttggatccttcgcctacccgccccgggctcaggtggctggctttcccgggcctggcgagttcttcccgccgcccgcgggtgcggagggctacccgcccgtggatggctaccctgcccctgacccgcgcgcggggctctacccagggccgcgcgaggactacgcattgcccgcggggttggaggtgtctgggaagctgagagtcgcgctcagcaaccacctgttgtggtccaagttcaaccagcaccagacagagatgatcatcactaagcaaggacggcgaatgttcccattcctgtccttcaccgtggccgggctggagcccacaagccattacaggatgtttgtggatgtggtcttggtggaccagcaccactggcggtaccagagcggcaagtgggtgcagtgtggaaaggcagaaggcagcatgccagggaaccgcttatatgtccacccagactcccccaacaccggagcccactggatgcgccaggaagtttcatttgggaagctaaagctcaccaacaacaagggggcttccaacaatgtgacccagatgatcgtcctgcagtctctccacaagtaccagccccggctgcacatcgtggaggtgaatgatggagagccagaggctgcctgcagtgcttctaacacacacgtctttactttccaagagacccagttcattgcagtgactgcctaccagaacgcagagatcactcagctgaaaatcgacaacaacccctttgccaaaggattccgggagaactttgagtccatgtacgcatctgttgatacgagtgtcccctcgccacctggacccaactgtcaactgcttgggggagaccccttctcacctcttctatccaaccagtatcctgttcccagccgtttctaccccgaccttccaggccagcccaaggatatgatctcacagccttactggctggggacacctcgggaacacagttatgaagcggagttccgagctgtgagcatgaagcccacactcctaccctctgccccggggcccactgtgccctactaccggggccaagacgtcctggcgcctggagctggttggcccgtggcccctcaatacccgcccaagatgagcccagctggctggttccggcccatgcgaactctgcccatggacccgggcctgggatcctcagaggaacagggctcctccccctcgctgtggcctgaggtcacctccctccagccggagcccagcgactcaggactaggcgaaggagacactaagaggaggaggatatccccctatccttccagtggcgacagctcctctcccgctggggccccttctccttttgataaggaaaccgaaggccagttttataattattttcccaactga

SEQ ID NO: 24 - protein, 530 aa, Mus musculus
MGIVEPGCGDMLTGTEPMPSDEGRGPGADQQHRFFYPEPGAQDPTDRRAGSSLGTPYSGGALVPAAPGRFLGSFAYPPRAQVAGFPGPGEFFPPPAGAEGYPPVDGYPAPDPRAGLYPGPREDYALPAGLEVSGKLRVALSNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACSASNTHVFTFQETQFIAVTAYQNAEITQLKIDNNPFAKGFRENFESMYASVDTSVPSPPGPNCQLLGGDPFSPLLSNQYPVPSRFYPDLPGQPKDMISQPYWLGTPREHSYEAEFRAVSMKPTLLPSAPGPTVPYYRGQDVLAPGAGWPVAPQYPPKMSPAGWFRPMRTLPMDPGLGSSEEQGSSPSLWPEVTSLQPEPSDSGLGEGDTKRRRISPYPSSGDSSSPAGAPSPFDKETEGQFYNYFPN

(19) United States
(12) Patent Application Publication
(10) Pub. No.: US 2009/0048117 A1
(43) Pub. Date: Feb. 19, 2009

(54) MODULATION OF IMMUNE SYSTEM FUNCTION BY MODULATION OF POLYPEPTIDE ARGININE METHYLTRANSFERASES

(75) Inventors: Laurie H. Glimcher, West Newton, MA (US); Kerri Mowen, San Diego, CA (US)

Correspondence Address: LAHIVE & COCKFIELD, LLP;FLOOR 30, SUITE 3000, ONE POST OFFICE SQUARE, BOSTON, MA, 02109, US

(73) Assignee: President and Fellows of Harvard College, Cambridge, MA (US)

(21) Appl. No.: 10/583,061

(22) PCT Filed: Dec. 20, 2004
(86) PCT No.: PCT/US2004/044095

§ 371 Date: Oct. 23, 2008

Related U.S. Application Data
(60) Provisional application No. 60/531,482, filed on Dec. 18, 2003.

(51) Int. Cl. C40B 30/04; C40B 30/08; C12Q 1/48; C12Q 1/68; C40B 30/06

(52) U.S. Cl. 506/9; 506/11; 506/10; 435/15; 435/6

(57) ABSTRACT

The instant invention pertains to, e.g., method of identifying a compound that modulates cytokine production or T cell receptor-mediated signaling, by identifying modulators of the expression and/or activity or PRMT polypeptides. The invention further pertains to methods for identifying a compound that modulates cytokine production in a non-T cell, by identifying compounds that modulate the expression and/or activity of NIP45. Methods for modulating cytokine production in cells by modulating the expression and/or activity of at least one molecule selected from the group consisting of: NIP45, PRMT1, and NFAT are also provided. The invention also pertains to methods for modulating the relative number of Th1 or Th2 cells is modulated and to methods of treating a subject that would benefit from the modulation of cytokine production comprising contacting an immune cell from the subject with an agent that modulates PRMT 1 expression and/or activity in the immune cell.

### RELATED APPLICATIONS

This application claims priority to U.S. Provisional Application Ser. No. 60/531,482, titled “Modulation of Immune System Function by Modulation of Polypeptide Arginine Methyltransferases” filed Dec. 18, 2003. This application is related to U.S. Ser. No. 10/448,748, titled “NFAT-INTERACTING PROTEIN NIP45 AND METHODS OF USE THEREFOR,” filed on May 30, 2003, now pending, which is a continuation of U.S. Ser. No. 09/617,923, titled “NFAT-INTERACTING PROTEIN NIP45 AND METHODS OF USE THEREFOR”, filed on Jul. 17, 2000, now U.S. Pat. No. 6,573,365 B1, which is a divisional of U.S. Ser. No. 09/192,611 titled “NFAT-INTERACTING PROTEIN NIP45 AND METHODS OF USE THEREFOR,” filed on Nov. 16, 1998, now U.S. Pat. No. 6,090,561, which is a divisional of U.S. Ser. No. 08/755,584 titled “NFAT-INTERACTING PROTEIN NIP45 AND METHODS OF USE THEREFOR,” filed on Nov. 25, 1996, now U.S. Pat. No. 5,858,711. This application is also related to U.S. Ser. No. 08/636,602; entitled “METHODS AND COMPOSITIONS FOR REGULATING T CELL SUBSETS BY MODULATING TRANSCRIPTION FACTOR ACTIVITY”, filed Apr. 23, 1996, and to a continuation-in-part application thereof, entitled “METHODS FOR REGULATING T CELL SUBSETS BY MODULATING TRANSCRIPTION FACTOR ACTIVITY”, U.S. Ser. No. 08/755,592, filed on Nov. 25, 1996. The entire contents of each of these applications is incorporated herein by reference.

### GOVERNMENT FUNDING

Work described herein was supported, at least in part, under grants AI37833 and AI43953 awarded by the National Institutes of Health. The U.S. government therefore may have certain rights in this invention.

### BACKGROUND OF THE INVENTION

CD4⁺ T helper cells are not a homogeneous population but can be divided on the basis of cytokine secretion into at least two subsets termed T helper type 1 (Th1) and T helper type 2 (Th2) (see e.g., Mosmann, T. R. et al. (1986) J. Immunol. 136:2348-2357; Paul, W. E. and Seder, R. A. (1994) Cell 76:241-251; Seder, R. A. and Paul, W. E. (1994) Ann. Rev. Immunol. 12:635-673). Th1 cells secrete interleukin-2 (IL-2) and interferon-γ (IFN-γ) while Th2 cells produce interleukin-4 (IL-4), Interleukin-5 (IL-5), interleukin-10 (IL-10) and interleukin-13 (IL-13). Both subsets produce cytokines such as tumor necrosis factor (TNF) and granulocyte/macrophage-colony stimulating factor (GM-CSF). In addition to their different pattern of cytokine expression, Th1 and Th2 cells are thought to have differing functional activities. For example, Th1 cells are involved in inducing delayed type hypersensitivity responses, whereas Th2 cells are involved in providing efficient “help” to B lymphocytes and stimulating production of IgG1 and IgE antibodies.

There is now abundant evidence that the ratio of Th1 to Th2 cells is highly relevant to the outcome of a wide array of immunologically-mediated clinical diseases including autoimmune, allergic and infectious diseases. For example, in experimental leishmania infections in mice, animals that are resistant to infection mount predominantly a Th1 response, whereas animals that are susceptible to progressive infection mount predominantly a Th2 response (Heinzel, F. P., et al. (1989) J. Exp. Med. 169:59-72; Locksley, R. M. and Scott, P. (1992) Immunoparasitology Today 1:A58-A61). In murine schistosomiasis, a Th1 to Th2 switch is observed coincident with the release of eggs into the tissues by female parasites and is associated with a worsening of the disease condition (Pearce, E. J., et al. (1991) J. Exp. Med. 173:159-166; Grzych, J-M., et al. (1991) J. Immunol. 141:1322-1327; Kullberg, M. C., et al. (1992) J. Immunol. 148:3264-3270). Many human diseases, including chronic infections (such as with human immunodeficiency virus (HIV) and tuberculosis) and certain metastatic carcinomas, also are characterized by a Th1 to Th2 switch (see e.g., Shearer, G. M. and Clerici, M. (1992) Prog. Chem. Immunol. 54:21-43; Clerici, M. and Shearer, G. M. (1993) Immunology Today 14:107-111; Yamamura, M., et al. (1993) J. Clin. Invest. 91:1005-1010; Pisa, P., et al. (1992) Proc. Natl. Acad. Sci. USA 89:7708-7712; Fauci, A. S. (1988) Science 239:617-623). Furthermore, certain autoimmune diseases have been shown to be associated with a predominant Th1 response. For example, patients with rheumatoid arthritis have predominantly Th1 cells in synovial tissue (Simon, A. K., et al. (1994) Proc. Natl. Acad. Sci. USA 91:8562-8566) and experimental autoimmune encephalomyelitis (EAE) can be induced by autoreactive Th1 cells (Kuchroo, V. K., et al. (1993) J. Immunol. 151:4371-4381).

The ability to alter or manipulate ratios of Th1 and Th2 subsets requires an understanding of the mechanisms by which the differentiation of CD4⁺ T helper precursor bells (Thp), which secrete only IL-2, choose to become Th1 or Th2 effector cells. It is clear that the cytokines themselves are potent Th cell inducers and form an autoregulatory loop (see e.g., Paul, W. E. and Seder, R. A. (1994) Cell 76:241-251; Seder, R. A. and Paul, W. B. (1994) Ann. Rev. Immunol. 12:635-673). Thus, IL-4 promotes the differentiation of Th2 cells while preventing the differentiation of precursors into Th1 cells, while IL-12 and IFN-γ have the opposite effect. One possible means therefore to alter Th1:Th2 ratios is to increase or decrease the level of selected cytokines. Direct administration of cytokines or antibodies to cytokines has been shown to have an effect on certain diseases mediated by either Th1 or Th2 cells. For example, administration of recombinant IL-4 or antibodies to IL-12 ameliorate EAE, a Th1-driven autoimmune disease (see Racke; M. K. et al. (1994) J. Exp. Med. 180:1961-1966; and Leonard, J. P. et al. (1995) J. Exp. Med. 181:381-386), while anti-IL-4 antibodies cure the Th2-mediated parasitic disease, Leishmania major (Sadick, M. D. et al. (1990) J. Exp. Med. 171:115-127). However, as therapeutic options, systemic administration of cytokines or antibodies may have unwanted side effects and, accordingly, alternative approaches to manipulating Th1/Th2 subsets are still needed.

While the molecular basis for the tissue-specific expression of T cell cytokines has remained elusive, study of the transcriptional elements of cytokine genes has provided insight into their regulation. Analysis of the IL-4 cytokine promoter, for example, has revealed functionally critical sites for several transcription factors including members of the NFAT and AP-1 families (Rooney, J. W. et al. (1995) Immunity 2:473-483; Szabo, S. J. et al. (1993) Mol. Cell. Biol. 13:4793-4805). NFAT is a multisubunit transcription complex that contains a cyclosporin A sensitive cytoplasmic phosphoprotein and an inducible nuclear component composed of AP-1 family member proteins (Flanagan, W. M. et al. (1991) Nature 352:803-807; Jain, J. et al. (1992) Nature 356:801-804). Purification and cloning of NFATp revealed a region of limited sequence identity to the Rel Homology Domain (RHD) of the NFκB family of transcription factors (McCaffrey, P. G. et al. (1993) Science 262:750-754). Subsequent cloning and sequencing of three related genes, NFATc, NFAT4/x/c3, and NFAT3/c4 revealed similar domains. NFAT family members share approximately 70% sequence similarity within this domain and approximately 18% sequence similarity to the RHD of the Rel/NFκB family of transcription factors. Consistent with their very limited sequence similarity in the RHD, there are marked differences in the behavior of NFκB and NFAT proteins, and much less is known about the pathways that mediate transcriptional regulation of NFAT target genes. However, considering that NFAT family members can bind to and transactivate the promoters of multiple cytokine genes including IL-2 and IL-4 (Rooney, J. et al. (1995) Immunity 2:545-553; Szabo, S. J. et al. (1993) Mol. Cell. Biol. 13:4793-4805; Flanagan, W. M. et al. (1991) Nature 352:803-807; Northrop, J. P. et al. (1994) Nature 369:497), NFAT proteins are not likely to be directly responsible for mediating Th1- or Th2-specific cytokine transcription.

Most, if not all, NFAT binding sites in cytokine promoter regulatory regions are accompanied by nearby sites that bind auxiliary transcription factors, usually members of the AP-1 family. It has been shown that NFAT and AP-1 proteins bind coordinately and cooperatively and are required for full activity of the IL-2 and IL-4 promoters. Different AP-1 proteins, specifically c-Jun, c-Fos, Fra-1, Fra-2, Jun B and Jun D, have been shown to bind to these sites (Rao, A. et al. (1994) Immunol. Today 15:274-281; Jain, J. et al. (1993) Nature 365:352-355; Boise, L. H. et al. (1993) Mol. Cell. Biol. 13:1911-1919; Rooney, J. et al. (1995) Immunity 2:545-553; Rooney, J. et al. (1995) Mol. Cell. Biol. 15:6299-6310). However, none of these AP-1 proteins is expressed in a Th1- or Th2-specific manner and there is no evidence for the differential recruitment of AP-1 family members to the IL-2 or IL-4 composite sites (Rooney, J. et al. (1995) Mol. Cell. Biol. 15:6299-6310). Thus, neither NFAT proteins nor the AP-1 family members c-Jun, c-Fos, Fra-1, Fra-2, Jun B and Jun D can account for the tissue-specific transcription of IL-4 in Th2 cells. NFAT has also been shown to be a regulator of IFN-γ production (Kiani et al. Blood. 2001 98:1480-8).

The proto-oncogene c-maf is expressed selectively in Th2 cells and is responsible for tissue-specific IL-4 expression. Interestingly, c-Maf acts in synergy with NEAT proteins to transactivate the IL-4 promoter. This is consistent with previous data showing that the inducible expression of multiple cytokine genes and cell surface proteins following T cell receptor stimulation requires members of the NFAT transcription factor family (Rooney, J. W. et al. (1995) Immunity 2:473-483; Cockerill, P. N. et al. (1995) Mol. Cell. Biol. 15:2071-2079; Goldfeld, A. E. et al. (1993) J. Exp. Med. 178:1365-1379; Shaw, J. P. et al. (1988) Science 241:202-205).

The identification of novel molecules involved in control of gene expression, particularly cytokine gene expression, in immune cells would be of great benefit.

### SUMMARY OF THE INVENTION

Post-translational modification adds a layer of complexity to the control of cytokine gene expression. Posttranslational modifications are often utilized to translate changes in the extracellular milieu into environment-sensitive gene expression in a timely and efficient fashion. Phosphorylation of serine, threonine, and tyrosine residues and protein ubiquitination have been widely studied (Roose, J. and Weiss, A. (2000) Nat Immunol 1:317-321). Although methylation of arginine residues was discovered over 30 years ago, it has only recently aroused substantial interest (McBride, A. E., and Silver, P. A. (2001) Cell 106: 5-8). The present invention is based, at least in part, on the finding that NIP45 is a target of arginine methyl transferases (PRMTs) and that NIP45 and PRMTs are involved in cytokine production in both T and non-T cells. As described in more detail below, activation of arginine methyltransferase (PRMT) in cells (e.g., T and non-T cells), results in the methylation of NIP45 which in turn leads to augmented cytokine production (e.g., IL-4 or IFN-γ production). Two types of arginine methyltransferases have been subclassified based on the symmetry of their reaction products. Both Type I (PRMT1, PRMT3, CARM1, PRMT6) and Type II (PRMT5) methyltransferases induce mono-methylation of arginine residues as a reaction intermediate, but type I protein arginine methyltransferases (PRMT) also generate asymmetric di-methylation of arginine residues and type II PRMTs catalyze the formation of symmetric dimethyl arginine residues (McBride, A. E., and Silver, P. A. (2001) Cell 106:5-8; Frankel, A., et al. (2002) J Biol Chem 277:3537-3543). Although isolated by sequence similarity with PRMT1, PRMT2 has not been demonstrated to have enzymatic activity (Scott, H. S., et al. (1998) Genomics 48: 330-340; Qi, C., et al. (2002). J Biol Chem 277:28624-28630). Arginine methylation has been shown to regulate subcellular localization (Shen, E. C, et al. (1998) Genes Dev 12:679-691; Yun, C. Y., and Fu, X. D. (2000) J Cell Biol 150:707-718) as well as to modulate protein-protein interactions. For example, arginine methylation of the proline-rich region of Sam68 prevents its interaction with its SH3 domain binding partners, Fyn, Lck, and Itk, without altering its affinity to WW domains (Bedford, M. T., et al. (2000) J Biol Chem 275: 16030-16036), while arginine methylation of a conserved arginine residue of Stat1 in response to IFNα/β signaling prevents interaction with its inhibitor PIAS1, thereby regulating Stat1 transcriptional ability (Mowen, K. A., et al. (2001) Cell 104: 731-741). PRMT1 deficient mice are embryonic lethal and CARM1 deficient mice die during late embryonic development or perinataly, suggesting a critical role for these PRMTs in cellular processes (Pawlak, M. R, et. al. (2002) J Cell Biochem 87: 394-407; Yadav, N., et al. (2003) Proc Natl Acad Sci USA 100: 6464-6468). Until the present invention, however, it was unknown how post-translational modification such as arginine methylation could affect the regulation of signal transduction and cytokine gene expression.

In one aspect, the invention pertains to a method of identifying a compound that modulates cytokine production, comprising: a) providing an indicator composition comprising a type I polypeptide arginine methyltransferase (PRMT1) polypeptide; b) contacting the indicator composition with a plurality of test compounds; c) selecting from the library of test compounds a compound of interest that modulates an activity of PRMT1; to thereby identify a compound that modulates cytokine production.

In another aspect, the invention pertains to a method of identifying a compound that modulates T cell receptor-mediated signaling, comprising: a) providing an indicator composition comprising a type I polypeptide arginine methyltransferase (PRMT1) polypeptide; b) contacting the indicator composition with a plurality of test compounds; c) selecting from the library of test compounds a compound of interest that modulates an activity of PRMT1; to thereby identify a compound that modulates T cell receptor-mediated signaling.

In one embodiment, the activity of PRMT1 is a NIP45-related activity. In another embodiment, the step of determining the effect of the compound of interest on a NIP45-related activity of PRMT1 comprises measuring cytokine production or cytokine gene transcription

In another aspect, the invention pertains to a method of identifying a compound that modulates cytokine production, comprising: a) providing an indicator composition comprising the upstream regulatory regions controlling expression of a type I polypeptide arginine methyltransferase (PRMT1) polypeptide operably linked to a reporter gene; b) contacting the indicator composition with a plurality of test compounds; c) selecting from the library of test compounds a compound of interest that modulates the expression of the reporter gene, to thereby identify a compound that modulates cytokine production.

In one embodiment the method further comprises determining the effect of the compound of interest on a NIP45-related activity of PRMT1. In another embodiment, the indicator composition is a cell that expresses the PRMT1 polypeptide. In one embodiment, the cell has been engineered to express the PRMT1 polypeptide by introducing into the cell an expression vector encoding the PRMT1 polypeptide. In yet another embodiment, the cell further comprises a NIP45 polypeptide. In still another embodiment, the cell further comprises an NFAT polypeptide.

In one embodiment, the cytokine is IFN-γ or IL-4.

In yet another embodiment the indicator composition is a cell free composition. In one embodiment, the step of determining the effect of the compound of interest on an activity of PRMT1 comprises measuring the ability of the PRMT1 to methylate one or more arginine residues of a target polypeptide. In one embodiment, the target polypeptide is NIP45.

In one aspect, the invention pertains to a method of identifying a compound that modulates cytokine production in a non-T cell, comprising; a) providing non-T cell comprising a NIP45 molecule; b) contacting the non-T cell with a plurality of test compounds; and c) selecting from the library of test compounds a compound of interest that modulates the activity of NIP45; to thereby identify a compound that modulates cytokine production in a non-T cell.

In one embodiment, the cell further comprises PRMT1. In another embodiment, the cell further comprises NFAT.

In one embodiment, the activity of NIP45 is selected from the group consisting of: binding to NFAT, binding to PRMT1, and activation of gene transcription. In one embodiment, the gene is selected from the group consisting of: IL-4, IFN-γ, Egr2, Egr3, c-Rel, and p65.

In another aspect, the invention pertains to a method of identifying a compound that modulates gene expression comprising: a) contacting an indicator composition comprising a first polypeptide comprising amino acids 1-32 of NIP45 and a second polypeptide which is a PRMT1 polypeptide with a plurality of test compounds; b) detecting an activity of the first polypeptide or a NIP45-related activity of the second polypeptide in the presence and absence of a test compound, and c) selecting a compound of interest that modulates an activity of the first or second polypeptide; to thereby identify a compound that modulates gene expression.

In one embodiment, the indicator composition is a cell. In one embodiment, the cell further comprises an NFAT polypeptide and the activity of the first polypeptide is detected by measuring the binding of the first polypeptide to the NFAT polypeptide.

In one embodiment, the NFAT polypeptide is selected from the group consisting of NFATc1, NFATc2, and NFATc3. In another embodiment, the activity of the first polypeptide is detected by measuring transcription from a promoter. In one embodiment, the promoter is an IL-4 or the IFN-γ promoter. In another embodiment, the promoter is selected from the group consisting of: the Egr2, Egr3, c-Rel, and p65 promoter.

In another embodiment, the indicator composition is present in a cell free system.

In yet another embodiment, the NIP45-related activity of the second polypeptide is detected by measuring the methylation of one or more arginine residues of NIP45.

In still another embodiment, the NIP45-related activity of the second polypeptide is detected by measuring the interaction between the second polypeptide and the first polypeptide.

In a further embodiment, the test compounds are present in a library of small molecules. In one embodiment, the test compound decreases the degree of arginine methylation of NIP45 as compared to the degree of arginine methylation of NIP45 in the absence of the test compound, and the test compound is identified as an agent that reduces cytokine production. In one embodiment, the test compound increases the degree of arginine methylation of NIP45 as compared to the degree of arginine methylation of NIP45 in the absence of the test compound, and the test compound is identified as an agent that increases cytokine production.

In another aspect, the invention pertains to a method of identifying a compound that modulates an interaction between NIP45 and a PRMT polypeptide, comprising: a) contacting an indicator composition comprising a polypeptide comprising amino acids 1-32 of NIP45 and a PRMT polypeptide with a plurality of test compounds; b) detecting a readout of the interaction between the NIP45 and PRMT polypeptides in the presence and absence of a test compound, and c) selecting a compound of interest that modulates the interaction between the NIP45 and PRMT polypeptides; to thereby identify a compound that modulates an interaction between NIP45 and PRMT polypeptide.

In one embodiment, the indicator composition is a cell based composition. In another embodiment, the indicator composition is a cell free composition.

In one embodiment, the readout of the interaction between the NIP45 and PRMT1 polypeptides is the binding of NIP45 to PRMT1 or the methylation of one or more arginine residues of NIP45. In another embodiment, the readout of the interaction between the first and second polypeptides is modulation of gene transcription. In one embodiment, the gene is selected from the group consisting of IL-4 and IFN-γ.

In one embodiment, the test compounds are present in a library of small molecules. In one embodiment, the test compound decreases the interaction between NIP45 and PRMT1 as compared to the interaction between NIP45 and PRMT1 in the absence of the test compound, and the test compound is identified as an agent that reduces interaction between NIP45 and PRMT1. In another embodiment, the test compound increases the interaction between NIP45 and PRMT1 as compared to the interaction between NIP45 and PRMT1 in the absence of the test compound, and the test compound is identified as an agent that increases interaction between NIP45 and PRMT1.

In yet another aspect, the invention pertains to a method of identifying a compound that modulates cytokine production in a cell, comprising; a) providing a cell containing one or more constructs which comprise: a cytokine promoter operably linked to a reporter gene, a nucleotide sequence encoding PRMT1, and a nucleotide sequence encoding at least one activator of cytokine gene transcription; b) stimulating the cell with an activating signal; c) contacting the cell with a plurality of test compounds; d) measuring the expression or activity of the reporter gene; and e) selecting a compound of interest that modulates the expression or activity of the reporter gene, to thereby identify a compound that modulates cytokine production in a cell.

In one embodiment, the cytokine promoter is an IFNγ promoter. In another embodiment, the cytokine promoter is an IL-4 promoter. In one embodiment, the activator of cytokine gene transcription is T-bet. In another embodiment, the activator of cytokine gene transcription is selected from the group consisting of NFATc2 and NIP45.

In one embodiment, the cell further comprises a construct comprising a nucleotide sequence encoding c-maf.

In yet another aspect, the invention pertains to a method of modulating cytokine production in a non-T cell comprising contacting a non-T cell with an agent that modulates the expression and/or activity of at least one molecule selected from the group consisting of: NIP45, PRMT1, and NFAT, such that cytokine production in the non-T cell is modulated.

In one embodiment, the cell is selected from the group consisting of: a dendritic cell, an NK cell, and a mast cell.

In yet another aspect, the invention pertains to a method of modulating cytokine production comprising contacting a T cell with an agent that modulates PRMT1 expression and/or activity such that cytokine production is modulated.

In one embodiment, the T cell is a CD4⁺ T cell. In another embodiment, the T cell is a CD8⁺ T cell.

In one embodiment, IFNγ production is modulated. In another embodiment, IL-4 production is modulated.

In one embodiment, PRMT1 activity is increased, thereby increasing j cytokine production. In another embodiment, PRMT1 activity is decreased, thereby decreasing cytokine production.

In another aspect, the invention pertains to a method of modulating IFNγ production, comprising contacting a cell with an agent that modulates PRMT1 expression and/or activity such that IFNγ production is modulated.

In one embodiment, the cell is ah NK cell or a dendritic cell.

In one aspect, the invention pertains to a method of modulating IL-4, comprising contacting a cell with an agent that modulates PRMT1 expression and/or activity such that IL-4 production is modulated.

In one embodiment, the cell is an NK cell or a mast cell.

In another aspect, the invention pertains to a method of modulating the relative number of Th1 or Th2 cell, comprising contacting a population of T cells with an agent that modulates PRMT1 activity such that the relative number of Th1 or Th2 cells is modulated.

In still another aspect, the invention pertains to a method of treating a subject that would benefit from the modulation of cytokine production comprising contacting an immune cell from the subject with an agent that modulates PRMT1 expression and/or activity in the immune cell such that cytokine production is modulated and the subject that would benefit from the modulation of cytokine production is treated.

In one embodiment, PRMT1 activity is increased, thereby increasing cytokine production. In a further embodiment, the patient is suffering from an immunodeficiency.

In another embodiment, PRMT1 activity is decreased, thereby decreasing cytokine production. In a further embodiment, the subject is suffering from an autoimmune or allergic condition.

### BRIEF DESCRIPTION OF THE DRAWINGS

FIG. 1 is photograph of yeast colonies, in triplicate, transformed with the NIP45 plasmid and either NFATp-RHD as/“bait” or control baits, Max, CDK2 or pEG202, together with the LacZ reporter plasmid pSH18, indicating that only those colonies containing the NIP45 plasmid and the NFATp-RHD bait expressed the LacZ reporter gene.

FIG. 2 is a photograph of an immunoprecipitation/Western blot experiment demonstrating that NIP45 and NFATp interact in HepG2 cells.

FIG. 3 is a schematic diagram comparing the structures of the original NIP45 cDNA clone isolated from the yeast two-hybrid screen (top) and the longest NIP45 cDNA clone isolated from a D10.G4 lambda zap II library (bottom).

FIG. 4 depicts the nucleotide and predicted amino acid sequences of the original NIP45 cDNA isolate.

FIG. 5 depicts the hydrophobicity plot of the NIP45 cDNA.

FIG. 6 is a photograph of an RNA blot analysis of NIP45 transcript levels in various tissues.

FIG. 7A is a photograph of immunofluorescence analysis of BHK cells transfected with an expression construct encoding an HA-epitope tagged NIP45 protein and probed with a monoclonal antibody specific for the HA peptide as the primary antibody and an indocarbocyanine labeled goat anti-mouse secondary reagent.

FIG. 7B is a photograph of the same cells depicted in FIG. 7A counterstained with the DNA staining dye Hoechst 33258.

FIG. 7C is a photograph of immunofluorescence analysis of unstimulated BHK cells transfected with an expression construct encoding NFAT4 and probed with an anti-NFAT4 specific antibody as the primary antibody and an indocarbocyanine labeled goat anti-mouse secondary reagent.

FIG. 7D is a photograph of the same cells depicted in FIG. 7C counterstained with the DNA staining dye Hoechst 33258.

FIG. 7E is a photograph of immunofluorescence analysis of ionomycin-treated BHK cells transfected with an expression construct encoding NFAT4 and probed with an anti-NFAT4 specific antibody as the primary antibody and an indocarbocyanine labeled goat anti-mouse secondary reagent.

FIG. 7F is a photograph of the same cells depicted in FIG. 7D counterstained with the DNA staining dye Hoechst 33258.

FIG. 8 depicts CAT assay results (left) and a bar graph quantitating the relative fold induction of CAT activity (right) in HepG2 cells transfected with a 3X NFAT-CAT reporter gene construct (containing three NFAT binding sites) and either a control expression plasmid or an NFAT family expression plasmid (NFATp, NFATc, NFAT3 or NFAT4), alone (−) or in combination with a NIP45 expression plasmid (⁺).

FIG. 9 depicts CAT assay results (left) and a bar graph quantitating the relative fold induction of CAT activity (right) in HepG2 cells transfected with an IL-4-CAT reporter gene, construct (extending to −732 bp of the IL-4 promoter) and combinations of NFATp, NIP45 and/or c-Maf expression constructs, as indicated.

FIG. 10 is a bar graph depicting the level of IL-4 (in pg/ml) in the supernatants of M12 B lymphoma cells transiently cotransfected with expression plasmids for NFATp, c-Maf and a pCI vector control (top bar) or expression plasmids for NFATp, c-Maf and NIP45 (bottom bar).

FIGS. 11A-11C depict the effects of MTA on T helper cytokine production. (A) T helper cells were isolated from the lymph nodes of DO11.10 TCR transgenic mice and grown under Th1 or Th2 conditions for 7 days. Th1 and Th2 cells were treated with PMA/ionomycin for 3 hrs or pretreated with 1 mM MTA for 60 min prior to PMA/ionomycin stimulation. RNA was isolated with analyzed by RNase protection analysis for transcript level of indicated cytokines. L32 and GAPDH included in the multitemplate probe set served as an internal control. (B) Cells were grown as in (A) either pretreated with 1 mM MTA or left untreated and intracellular cytokine analysis was performed after stimulation with PMA/ionomycin for 2 hrs and an additional 2 hrs with 3 mM monensin. (C) MTA inhibits IL-4 promoter activity. Jurkat cells were transfected with IL-4 reporter vector (2.5 μg) and expression vectors for c-Maf (2.5 μg), NFATc2 (2.5 μg), and PRMT1 (10 μg) along with a TK-renilla luciferase vector (5 ng) as an internal control. Transfectants were left unstimulated or pretreated with 1 mM MTA before a 6 hr PMA/ionomycin stimulation. Luciferase values were calculated relative to TK renilla luciferase internal controls and are expressed relative to unstimulated reporter activity. Similar results were obtained in at least three independent experiments.

FIGS. 12A-12C show that T helper cells highly express PRMT1. (A) PRMT expression in T helper cells. Thp, Th1, Th2, and 293 cell protein lysates (30 μg) were analyzed by western blot with antibodies to PRMT1, PRMT2, PRMT3, CARM1, PRMT5, and PRMT6. The location of PRMT6 is indicated by an arrow. Equal loading was confirmed using an antibody to HSP90. (B) PRMT1 transcripts are upregulated by TCR stimulation. T helper cells from DO11.10 TCR transgenic mice were unstimulated or stimulated with plate-bound anti-CD3 and anti-CD28 for 1 days or 3 days. On day 3, cells were split with into fresh media with IL-2 until day 5 or day 6. For D6R samples, cells from day 5 were restimulated with plate-bound anti-CD3 for 24 hrs. RNA was prepared and analyzed by northern blot with a probe to PRMT1. Northern blots were reprobed with a β-actin probe to assess loading. (C) TCR induced PRMT1 expression is rapid and cyclosporin A (CsA) sensitive. T helper cells isolated from the lymph nodes of DO11.10 TCR transgenic mice were untreated or pretreated with CsA for 1 hr prior to stimulation with anti-CD3 and anti-CD28 for 0, 3, and 6 hours. RNA was harvested and Northern blot analysis for PRMT1 expression was performed. Equal loading was determined by reprobing with a β-actin probe.

FIGS. 13A-13D depict the regulation of the IFNγ and IL-4 promoters by PRMT1. (A) Jurkat cells were transfected with the 9.2 kb IFNγ luciferase reporter 5 μg) and expression vectors for T-bet (5 μg) and PRMT1 (10 μg). Cells were unstimulated or stimulated with PMA/ionomycin for 6 hrs prior to luciferase assays. (B) Jurkat cells were transfected with the IL-4 luciferase reporter (2.5 μg) and expression vectors for NFATc2 (2.5 μg), c-Maf (2.5 μg), and PRMT1 (10 μg). Cells were treated as in (A). (C) Jurkat cells were transfected with the IL-4 luciferase reporter (2.5 μg) and expression vectors for JunB (2.5 μg), c-Maf (2.5 μg), and PRMT1 (10 μg). Cells were treated as in (A). (D) Jurkat cells were transfected with an luciferase reporter driven by NFAT consensus sites and expression vectors for NFATc2 (2.5 μg) and PRMT1 (10 μg). Cells were treated as in (A). (Luciferase units were normalized to TK-renilla luciferase activity. Results are representative to at least three independent experiments.)

FIGS. 14A-14C show that NIP45 also augments transcription from IFNγ and IL-4 promoters. (A) Jurkat cells were transfected with the 9.2 kb IFNγ luciferase reporter (5 μg) and expression vectors for T-bet (5 μg) and NIP45 (5 μg) or with the IL-4 luciferase reporter (2.5 μg) and expression vectors for NFATc2 (2.5 μg), c-Maf (2.5 μg), and NIP45 (5 μg). Cells were unstimulated or stimulated with PMA/ionomycin for 6 hrs prior to luciferase assays. (Luciferase units were normalized to TK-renilla luciferase activity. Results are representative to at least three independent experiments.) (B) The amino terminus (a.a. 6-32) of NIP45 contains 11 arginine residues within consensus motifs for arginine methylation. Putative methylated arginines are indicated in bold. (C) NIP45 is methylated but ΔN-NIP45, NFATc2, T-bet, and c-Maf are not 293 cells were transfected with expression vectors for NIP45, ΔN-NIP45, NFATc2, T-bet, and c-Maf and were immunoprecipitated from cell extracts. Immunoprecipitates were subjected to in vitro methylation by recombinant PRMT1. Westerns were performed on immunoprecipitate samples to confirm expression. c-Maf expression is indicated by an arrow.

FIGS. 15A-15D show that NIP45 is methylated by PRMT1. (A) PRMT1, PRMT3, CARM1, and PRMT 5 proteins were immunoprecipitated from 293 cell extracts using anti-Flag agarose. These proteins were used for in vitro methylation assays with recombinant GST-NIP45. Western blot analysis of the Flag epitope tagged expression constructs was performed with a monoclonal antibody against the Flag epitope. (B) PRMT1-His and GST-CARM1 were used in in vitro methylation assays with GST, GST-NIP45, and GST-ΔN-NIP45 (GST-ΔN) and transferred to PVDF membrane. Histone H3 was used as a positive control for CARM1 activity and the input was verified by probing the PVDF with a polyclonal GST antibody. (C) Wild-type ES cells and PRMT 1−/− ES cells were transfected with control vector or Flag-tagged NIP45. Lysates were immunoprecipitated with anti-Flag agarose. Resolved proteins were immunoblotted with a polyclonal anti-dimethyl-arginine (asymmetric) antibody. Immunoblots were restripped and probed with a monoclonal antibody against the Flag epitope to determine equal loading. (D) Th1 and Th2 lysates from day 7 differentiation cultures were immunoprecipitated with a mixture of two monoclonal antibodies against NIP45 or the appropriate isotype control. Immunoblots were probed a polyclonal anti-dimethyl-arginine (asymmetric) antibody and were reprobed with a polyclonal NIP45 antibody to determine equal loading.

FIGS. 16A-16C depict the interaction of NIP45 and PRMT. (A) Jurkat cell lysates were used in pulldown assays with 25 μg of recombinant GST, GST-NIP45, and GST-ΔN-NIP45. The presence of PRMT1 or CARM1 in pulldown samples was determined by immunoblotting with a monoclonal PRMT1 antibody and a polyclonal CARM1 antibody. PVDF membranes were reprobed with anti-GST antibodies to determine equal amount of recombinant proteins in pulldown samples. (B) Flag-NIP45or Flag-ΔN-NIP45 or vector control and HA-PRMT1 expression constructs were transfected in 293 cells. Lysates were immunoprecipitated with anti-Flag agarose and interaction with PRMT1 was determined by probing immunoblots with a monoclonal anti-HA antibody. Equal loading was assessed by probing with an anti-Flag antibody. (C) Lysates from unstimulated or 60 min PMA/ionomycin stimulated day 7 Th1 or Th2 differentiation cultures were immunoprecipitated with a monoclonal PRMT1 antibody or appropriate isotype control. Interaction with endogenous NIP45 was determined by immunoblotting PRMT1 antibody to determine equal loading.

FIGS. 17A-17B show that MTA inhibits NIP45 and NFATc2 interaction. (A) Jurkat cells were transfected with the IL-4 promoter (2.5 μg) and expression vectors encoding NFATc2 (2.5 μg), c-Maf (2.5 μg), NIP45 (5 μg), ΔN-NIP45 (5 μg), and PRMT1 (10 μg). Cells were unstimulated or stimulated with PMA/ionomycin for 6 hrs-prior to luciferase assays. (Luciferase units were normalized to TK-renilla luciferase activity. Results are representative to at least three independent experiments.) (B) 293 cells were transfected with HA-NFATc2 and NIP45-MycHis expression vectors and left untreated or treated with 1 mM MTA. Lysates were immunoprecipitated with anti-Myc agarose or isotype control. Immunoblots were probed with a monoclonal anti-HA antibody and reprobed with a monoclonal anti-Myc (9E10) antibody to determine equal loading.

FIGS. 18A-18D show that, NIP45 deficient mice have defects in Th cell cytokine production as well as non-T cell cytokine production.

### DETAILED DESCRIPTION OF THE INVENTION

This invention is based, at least in part, on the finding that NIP45 is a target of arginine methyl transferases (PRMTs) and that NIP45 and PRMTs are involved in cytokine production in both T and non-T cells. NIP45 (NFAT Interacting Protein 45) is a 45 kDa protein that interacts with NFAT proteins. A cDNA encoding NIP45 was isolated based upon the interaction of NIP45 with the Rel homology domain (RHD) of NFATp using a two-hybrid interaction trap assay in yeast (see Example 1). Coimmunoprecipitation experiments demonstrated that NIP45 and NFAT interact in vivo in mammalian cells (see Example 2). The cDNA encoding NIP45 has been sequenced and characterized (see Example 3). Examination of the tissue expression pattern of NIP45 mRNA revealed that the NIP45 transcript is preferentially expressed in spleen, thymus and testis (see Example 4). Subcellular localization studies demonstrated that NIP45 protein is evenly distributed throughout the cell nucleus (see Example 5). Functional studies showed that NIP45 synergizes with NFAT to stimulate transcription from promoters containing NFAT binding sites and, moreover, synergizes with NFAT and c-Maf to stimulate transcription from the IL-4 promoter (see Example 6). Moreover, NIP45, NFAT and c-Maf can act in concert to induce expression of the endogenous IL-4 gene in cells that do not normally express IL-4 (e.g., B cells.) (see Example 7).

In addition, the present invention demonstrates that arginine methylation plays an important role in cytokine production (Example 8). PRMT1 is the predominantly expressed arginine methyltransferase in T helper cells, and expression of PRMT1 is induced by TCR stimulation (Example 9). PRMT1 activates the IFN-γ and IL-4 promoters (Example 10), and one target of PRMT1 activity is the amino-terminus of the NFAT interacting protein, NIP45. Arginine methylation of NIP45 acts to modulate the interaction between NIP45 and NFAT, resulting in alterations of cytokine gene expression (Examples 11-14).

So that the invention may be more readily understood, certain terms are first defined.

### I. DEFINITIONS

As used herein, the term “NFAT family protein” (also referred to as simply “NFAT”) refers to the family of Nuclear Factors of Activated T cell transcription factors, including NFATp, NFATc, NFAT4/x/3′ and NFAT3/c4.

As used herein the term “Rel Homology Domain of an NFAT family protein” (abbreviated as RHD domain) refers to a domain within NFAT family proteins having approximately 70% sequence similarity within the RHD of the Rel/NFκB family of transcription factors.

As used herein a “NIP45-interacting molecule” or “NIP45-binding molecule”, used interchangeably herein, includes molecules, e.g., a polypeptide, that interacts with NIP45. Non-limiting examples of NIP45-interacting molecules are PRMTs, e.g. PRMT1, and NFAT family members.

The term “interact” as used herein is meant to include detectable interactions between molecules, such as can be detected using, for example, a yeast two hybrid assay or coimmunoprecipitation. The term interact is also meant to include “binding” interactions between molecules. Interactions may be protein-protein or protein-nucleic acid in nature.

As used herein, “PRMT”, includes e.g., PRMT1, PRMT2, PRMT3, PRMT5, PRMT6, and CARM1. In a preferred embodiment, the term “PRMT” refers to PRMT1. PRMTs are polypeptide arginine methyltransferases that induce methylation, e.g., monomethylation, of arginine residues as a reaction intermediate. PRMTs catalyze the sequential transfer of a methyl group from S-adenosylmethionene to the side chain nitrogens of arginine residues within proteins to form methylated arginine derivatives and S-adenosyl-L-homocysteine. PRMTs are categorized as Type I (e.g., PRMT 1, PRMT3, PRMT6, and CARM1) and Type 2 (e.g., PRMT). Type I protein arginine methyltransferases generate asymmetric dimethylation of arginine residues; type II protein arginine methyltransferases generate symmetric dimethyl arginine residues ((McBride, A. E., and Silver, P. A. (2001) Cell 106:5-8, Frankel, A., et al. (2002) J Biol Chem 277: 3537-3543). PRMTs are also described, e.g., in USSN 20030017489.

As used herein the term “PRMT activity” or “PRMT biological activity” includes modulation of methylation of at least one arginine residue on a polypeptide.

As used herein, the term “NIP45-related activity of PRMT” includes one or more of the following: the ability of a PRMT to bind to NIP45, the ability of a PRMT to modulate binding of NIP45 to NFAT family proteins, the ability of a PRMT to methylate at least one arginine residue of NIP45, the ability to modulate (e.g., inhibit or enhance) cytokine production (for example, T cell receptor (TCR) initiated cytokine production or cytokine production by non-T cells such as mast cells), or the ability to modulate various immune responses downstream of cytokine production (e.g., the ability to modulate immune cell effector function, the ability to modulate the relative number of Th1 or Th2 cells).

As used herein, the various forms of the term “modulate” include stimulation (e.g., increasing or upregulating a particular response or activity) and inhibition (e.g., decreasing or downregulating a particular response or activity).

As used herein, the term “contacting” (i.e., contacting a cell e.g. an immune cell, with an compound) is intended to include incubating the compound and the cell together in vitro (e.g., adding the compound to cells in culture) or administering the compound to a subject such that the compound and cells of the subject are contacted in vivo. The term “contacting” is not intended to include exposure of cells to a modulator or compound that may occur naturally in a subject (i.e., exposure that may occur as a result of a natural physiological process).

As used herein the term “upstream regulatory regions” or “upstream regulatory sequences” includes those sequences 5′ of a gene, e.g., promoters and/or enhancers, that control transcription of the gene. Upstream regulatory regions of many genes are known in the art and one of ordinary skill in the art can identify such regions.

For example, given the sequence of genomic DNA upstream of, for example PRMT1 available at GI:38083885, one of ordinary skill in the art can identify promoters and/or enhancers of PRMT.

As used herein, the term “test compound” includes a compound that has not previously been identified as, or recognized to be one or more of the following: a modulator of the activity of a NIP45-interacting molecule, e.g., PRMT, in immune cells; a modulator of a NIP45-related activity of PRMT; or a modulator of the interaction between NIP45 and a NIP45 interacting polypeptide, e.g., PRMT.

“T-bet” (T box expressed in T cells) is a member of the T box family of transcription factors whose founding member is the brachyury gene). T-box proteins comprise a T box domain which binds to DNA at a T box binding site. Different cell types and different genes respond to T-bet, which serves to modulate a variety of cellular responses. T-bet is constitutively expressed selectively in thymocytes and Th1 cells. T-bet acts by promoting the Th1 phenotype in naïve T helper precursor cells (Thp), both by initiating Th1 cell genetic programs and by repressing the opposing programs in Th2 cells. T-bet accomplishes the former by directly driving the transcription of the IFNγ gene as well as the IL-12Rβ2 chain. T-bet also controls IFN-γ production in CD8+ T cells, as well as in cells of the innate immune system, e.g., NK cells and dendritic cells. See, e.g., Szabo, S. J., et al. (2000) Cell 100(6):655-69; Szabo, S. J., et al. (2002) Science 295(5553):338-42; Peng, S. L., et al. (2002) Proc Natl Acad Sci USA. 99(8):5545-50; and Glimcher, L. H., et al. (2004) Nat Rev Immunol. 4(11):900-11.

The term “library of test compounds” is intended to refer to a panel comprising a multiplicity of test compounds.

As used herein, the term “cell free composition” refers to an isolated composition which does not contain intact cells. Examples of cell free compositions include cell extracts and compositions containing isolated proteins.

As used herein, an “antisense” nucleic acid molecule comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a nucleic acid molecule of the invention is a compound that mediates RNA interference, RNAi. RNA interfering agents include, but are not limited to, nucleic acid molecules including RNA molecules which are homologous to the target gene or genomic sequence, e.g., NIP45 or a NIP45-interacting molecule, or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries known in the art for use in antisense RNA can be employed.

As used herein, the term “immune response” includes immune cell-mediated (e.g., T cell and/or B cell-mediated) immune responses that are influenced by modulation of immune cell activation. Exemplary immune responses include B cell responses (e.g., antibody production), T cell responses (e.g., proliferation, cytokine production and cellular cytotoxicity), and activation of cytokine responsive cells, e.g., macrophages. In a preferred embodiment of the invention, an immune response is T cell mediated. As used herein, the term “downregulation” with reference to the immune response includes a diminution in one or more immune responses (e.g., modulation of T cell-mediated immune responses) while the term “upregulation” with reference to the immune response includes an increase in any one or more immune responses, e.g., T cell responses. It will be understood that upregulation of one type of immune response may lead to a corresponding downregulation in another type of immune response. For example, upregulation of the production of certain cytokines (e.g., IL-10) can lead to downregulation of cellular immune responses.

As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes.

As used herein, the term “T cell” includes CD4⁺ T cells and CD8⁺ T cells. The term T cell also includes both T helper 1 (Th1) type T cells and T helper 2 (Th2) type T cells, also referred to herein as “effector T cells”. The terms “antigen presenting cell” and “APC”, as used interchangeably herein, include professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

As used herein, the term “receptor” includes immune cell receptors that bind antigen, complexed antigen (e.g., in the context of MHC molecules), or antibodies. Activating receptors include, e.g., T cell receptors (TCRs), B cell receptors (BCRs), cytokine receptors, LPS receptors, complement receptors, and Fc receptors. For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

As used herein, the term “dominant negative” includes polypeptide molecules e.g., portions or variants thereof) that compete with native (i.e., wild-type) polypeptide molecules, but which compete with the native polypeptide and lack at least one activity of the native polypeptide, thereby downmodulating the activity of the native polypeptide.

As used herein, the term “nucleic acid molecule” includes DNA molecules (e.g., cDNA or genomic DNA) and RNA molecules (e.g., mRNA). The nucleic acid molecule may be single-stranded or double-stranded, but preferably is double-stranded DNA.

As used herein, the term “nucleic acid” includes wild-type nucleic acid molecules or fragments or equivalents thereof (e.g., fragments or equivalents of NIP45or NIP45-interacting molecules, e.g., PRMT or NFAT). The nucleotide sequences of the wild-type NIP45, NFATc1, M7, NFATc2, NFATc3 and PRMT1 are known in the art and described in, for example, Mak, C. H., et al. (1998) Immunogenetics 48:32-39, Pan, S., et al. (1997) Biochem Biophys Res Comm 240:314-323, McCaffrey, P. G., et al. (1993) Science 262:750-754, Ho, S. N., et al. (1995) J Biol Chem 270:19898-19907, and Pawlak, M. R., et al. (2000) Mol Cell Biol 20:4859-4869, respectively, the contents of which are incorporated herein by reference. The amino acid sequence of NIP45 protein has been determined (shown in SEQ ID NO: 2) and a cDNA encoding NIP45 protein has been isolated (the nucleotide sequence of which is shown in SEQ ID NO: 1). The nucleotide sequence of NFATc1 is shown in SEQ ID NO:3, the amino acid sequence in SEQ ID NO:4. The nucleotide sequence of NFATc2 is shown in SEQ ID NO:5, the amino acid sequence in SEQ ID NO:6. The nucleotide sequence of NFATc3 is shown in SEQ ID NO:7, the amino acid sequence in SEQ ID NO:8. The nucleotide sequence of PRMT1 is shown in SEQ ID NO:9, the amino acid sequence in SEQ ED NO:10. The nucleotide sequence of T-bet is shown in SEQ ID NO:23, the amino acid sequence in SEQ ID NO:24.

As used herein, a “naturally-occurring” nucleic acid molecule refers to an RNA or DNA molecule having a nucleotide sequence that occurs in nature (e.g., encodes a natural protein).

The term “equivalent” is intended to include nucleotide sequences encoding polypeptides that are functionally equivalent (e.g., to NIP45 or NIP45-interacting molecule proteins) i.e., proteins which maintain at least one biological activity of the native nucleic acid molecule. In one embodiment, a functionally equivalent NIP45 amino acid molecule has ability to interact with NFAT, in particular the NFAT Rel homology domain. In another embodiment, NIP45 has the ability to interact with PRMT. In another preferred embodiment, a functionally equivalent NIP45-interacting molecule has the ability to bind NIP45 in an immune cell, e.g., a T cell. In another preferred embodiment, a functionally equivalent PRMT protein has at least one PRMT biological activity, preferably at least one NIP45-related biological activity, e.g., the ability to modulate the binding of NIP45 and NFAT in an immune cell, e.g., a T cell.

An used herein, an “isolated nucleic acid molecule”, refers to a nucleic acid molecule that is free of gene sequences which naturally flank the nucleic acid in the genomic DNA of the organism from which the nucleic acid is derived (i.e., gene sequences that are located adjacent to the isolated nucleic molecule in the genomic DNA of the organism from which the nucleic acid is derived). For example, in various embodiments, an isolated NIP45 or NIP45-interacting molecule nucleic acid molecule may contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, may be free of other cellular material.

As used herein, the term: “hybridizes under stringent conditions” is intended to describe conditions for hybridization and washing under which nucleotide sequences at least 60% homologous to leach other typically remain hybridized to each other. Preferably, the conditions are such that at least sequences at least 65%, more preferably at least 70%, even more preferably at least 75%, and yet more preferably 80% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, an “isolated protein” or “isolated polypeptide” refers to a protein or polypeptide that is substantially free of other proteins, polypeptides, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the protein is derived, or substantially free from chemical precursors or other chemicals when chemically synthesized. The language “substantially free of cellular material” includes preparations of, for example, NIP45 or NIP45-interacting molecules, protein in which the protein is separated from cellular components of the cells from which it is isolated or recombinantly produced.

The nucleic acids of the invention can be prepared by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “transgenic cell” refers to a cell containing a transgene.

As used herein, the term “transgenic animal” refers to a non-human animal, e.g., a swine, a monkey, a goat, or a rodent, e.g., a mouse, in which one or more, and preferably essentially all, of the cells of the animal include a transgene, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, the term “rodent” refers to all members of the phylogenetic order Rodentia.

As used herein, a “homologous recombinant animal” refers to a type of transgenic non-human animal, preferably a mammal, more preferably a mouse, in which an endogenous gene, e.g., NIP45 or a NIP 45-interacting molecule, has been altered by homologous recombination between the endogenous gene and an exogenous DNA molecule introduced into a cell of the animal, e.g., an embryonic cell of the animal, prior to development of the animal.

As used herein, the term “misexpression” includes a non-wild-type pattern of gene expression. Expression as used herein includes transcriptional, post transcriptional e.g., mRNA stability, translation, and post translational stages. Misexpression includes: expression at non-wild-type levels, i.e., over or under expression; a pattern of expression that differs from wild-type in terms of the time or stage at which the gene is expressed, increased or decreased expression (as compared with wild-type) at a predetermined developmental period or stage; a pattern of expression that differs from wild-type in terms of decreased expression (as compared with wild-type) in a predetermined cell type or tissue type; a pattern of expression that differs from wild-type in terms of the splicing size, amino acid sequence, post-translational modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus. Misexpression includes any expression from a transgenic nucleic acid. Misexpression includes the lack or non-expression of a gene or transgene, e.g., that can be induced by a deletion of all or part of the gene or its control sequences.

As used herein, the term “knockout” refers to an animal or cell therefrom, in which the insertion of a transgene disrupts an endogenous gene in the animal or cell therefrom. For example, the disruption can essentially eliminate NIP45 or a NIP45-interacting molecule in the animal or cell. In preferred embodiments, misexpression of the gene encoding the NIP45 or a NIP45-interacting protein is caused by disruption of the gene encoding NIP45 or a NIP45-interacting molecule. For example, the gene can be disrupted through removal of DNA encoding all or part of the protein.

In preferred embodiments, the animal can be heterozygous or homozygous for a misexpressed gene, e.g., it can be transgenic animal heterozygous or homozygous for a transgene encoding NIP45 or a NIP45-interacting molecule.

In preferred embodiments, the animal is a transgenic mouse with a transgenic disruption of the gene encoding NIP45 or a NIP45-interacting molecule, preferably an insertion or deletion, which inactivates the gene product.

In another aspect, the invention features, a nucleic acid molecule which, when introduced into an animal or cell, results in the misexpression of the NIP45 or a NIP45-interacting molecule gene in the animal or cell. In preferred embodiments, the nucleic acid molecule, includes an NIP45 or a NIP45-interacting molecule nucleotide sequence which includes a disruption, e.g., an insertion or deletion and preferably the insertion of a marker sequence.

As used herein, the term “marker sequence” includes a nucleic acid molecule that (a) is used as part of a nucleic acid construct (e.g., the targeting construct) to disrupt the expression of the gene of interest (e.g., the NIP45 or a NIP45-interacting molecule) and (b) is used to identify those cells that have incorporated the targeting construct into their genome. For example, the marker sequence can be a sequence encoding a protein which confers a detectable trait on the cell, such as an antibiotic resistance gene, e.g., neomycin resistance gene, or an assayable enzyme not typically found in the cell, e.g., alkaline phosphatase, horseradish peroxidase, luciferase, beta-galactosidase and the like.

As used herein, “disruption of a gene” refers to a change in the gene sequence, e.g., a change in the coding region. Disruption includes: insertions, deletions, point mutations, and rearrangements, e.g., inversions. The disruption can occur in a region of the native NIP45 or a NIP45-interacting molecule DNA sequence (e.g., one or more exons) and/or the promoter region of the gene so as to decrease or prevent expression of the gene in a cell as compared to the wild-type or naturally occurring sequence of the gene. The “disruption” can be induced by classical random mutation or by site directed methods. Disruptions can be transgenically introduced. The deletion of an entire gene is a disruption. Preferred disruptions reduce NIP45 or a NIP45-interacting molecule levels to about 50% of wild-type, in heterozygotes or essentially eliminate NIP45 or a NIP45-interacting molecule in homozygotes.

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. Preferably, antibodies of the invention bind specifically or substantially specifically to NIP45 or a NIP45-interacting molecule, e.g., PRMT or NFAT molecules, (i.e., have little to no cross reactivity with non-PRMT, non-NIP45, or non-NFAT molecules). The terms “monoclonal antibody” and “monoclonal antibody composition”, as used herein, refer to a population of antibody molecules that contain only one species of an antigen binding site capable of immunoreacting with a particular epitope of an antigen. A monoclonal antibody composition thus typically displays a single binding affinity for a particular antigen with which it immunoreacts.

In one embodiment, small molecules may be used as test compounds. The term “small molecule” is a term of the art and includes molecules that are less than about 7500, less than about 5000, less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., Cane et. al. 1998. Science 282:63), and natural product extract libraries. In another embodiment, the compounds are small, organic non-peptidic compounds. In a further embodiment, a small molecule is not biosynthetic. For example, a small molecule is preferably not itself the product of transcription or translation.

Various aspects of the invention, are described in further detail in the following subsections:

### II. SCREENING ASSAYS

The invention provides methods also referred to herein as “screening assays”) for identifying modulators, i.e., candidate or test compounds or agents (e.g., peptidomimetics, small molecules or other drugs) which modulate, for example one or more of NIP45 activity (e.g., the ability to bind to NFAT; the ability to modulate cytokine production in an immune cell (e.g., a T cell or a non-T cell, such as an NK cell or a mast cell) or the ability to bind to PRMT); PRMT activity in an immune cell (e.g., methylation of at least one arginine residue on a polypeptide; modulation of gene transcription); NIP45-related PRMT activity (e.g., methylation of at least one residue on NIP45; modulation of the ability of NIP45 to bind to NFAT; modulation of cytokine production) or for testing or optimizing the activity of such agents.

The assays can be used to identify agents that modulate the function of NIP45 and/or a NIP45-binding molecule. For example, such agents may interact with NIP45 or the NIP45-binding molecule (e.g., to inhibit or enhance their activity). The function of NIP45 or the NIP45-binding molecule can be affected at any level, including transcription, protein expression, protein localization, and/or cellular activity. The subject assays can also be used to identify, e.g., agents that alter the interaction of NIP45or the NIP45-binding molecule with a binding partner, substrate, or cofactors.

The subject screening assays can measure the activity of NIP45 or a NIP45-binding protein directly (e.g., arginine methylation of NIP45, binding of NIP45 to a NIP45-binding protein such PRMT or NFAT, activation of gene transcription by NIP45), or can measure a downstream event controlled by modulation of NIP45 or a NIP45-binding protein (e.g., by measuring the amount of cytokine produced by a cell or by measuring an effect of such cytokine production on a cell or organism).

The subject screening assays employ indicator compositions. These indicator compositions comprise the components required for performing an assay that detects and/or measures a particular event. The indicator compositions of the invention provide a reference readout and changes in the readout can be monitored in the presence of one or more test compounds. A difference in the readout in the presence and the absence of the compound indicates that the test compound is a modulator of the molecule(s) present in the indicator composition.

The indicator composition used in the screening assay can be a cell that expresses a NIP45 polypeptide or a NIP45-binding molecule, e.g., the PRMT1, protein. For example, a cell that naturally expresses or, more preferably, a cell that has been engineered to express the protein by introducing into the cell an expression vector encoding the protein may be used. Preferably, the cell is a mammalian cell, e.g., a human cell. In one embodiment, the cell is a T cell. In another embodiment, the cell is a non-T cell. Alternatively, the indicator composition can be a cell-free composition that includes the protein (e.g., a cell extract or a composition that includes e.g., either purified natural or recombinant protein).

In another embodiment, the indicator composition comprises more than one polypeptide. For example, in one embodiment the subject assays are performed in the presence of NIP45 and at least one NIP45-binding molecule, e.g., PRMT. The indicator composition may further comprise a gene encoding at least one activator of cytokine gene transcription, e.g., T-bet or an NFAT polypeptide, e.g., NFAtc1, NFATc2. In another embodiment, the indicator composition may further comprise a maf gene. In an exemplary embodiment, an indicator composition comprises expression vector(s) encoding PRMT, NIP45, NFATc2 and c-maf.

Compounds that modulate the expression and/or activity of NIP45 and/or a NIP45 binding molecule, e.g., PRMT, identified using the assays described herein can be useful for treating a subject that would benefit from the modulation of cytokine production. Exemplary conditions that can benefit from modulation of cytokine production include autoimmune disorders as well as immunodeficiency disorders.

The subject screening assays can be performed in the presence or absence of other agents. In one embodiment, the subject assays are performed in the presence of an agent that provides a stimulatory signal to a cell. For example, in one embodiment, assays are performed in the presence of an agent that delivers (e.g., an antibody that recognizes the T cell receptor or an associated molecule) or mimics a T cell receptor-mediated signal (e.g., PMA and Ionomycin). In another embodiment, the screening assays of the invention are performed in the presence of an agent that inhibits the activity of a component of the assay. For example, in one embodiment, the assays are performed in the presence of 5′-methyl-thioadenosine (MTA), an agent that specifically inhibits protein methyltransferase activity.

In one embodiment, secondary assays can be used to confirm that the modulating agent effects a PRMT molecule, or an NFAT molecule in a NIP45 related manner. For example, compounds identified in a primary screening assay can be used in a secondary screening assay to determine whether the compound affects a NIP45-related activity. Accordingly, in another aspect, the invention pertains to a combination of two or more of the assays described herein. For example, a modulating agent can be identified using a cell-based or a cell-free assay, and the ability of the agent to modulate the activity of PRMT can be confirmed in vivo, e.g., in an animal such as, for example, in an animal model of a disorder or a NIP45 transgenic animal.

Moreover, a modulator of cytokine production, expression and/or activity identified as described herein (e.g., an antisense nucleic acid molecule, or a specific antibody, or a small molecule) may be used in an animal model to determine the efficacy, toxicity, or side effects of treatment with such a modulator. Alternatively, a modulator identified as described herein may be used in an animal model to determine the mechanism of action of such a modulator.

In one embodiment, the screening assays of the invention are high throughput or ultra high throughput (e.g., Fernandes P B, Curr Opin Chem Biol. 1998 2:597; Sundberg S A, Curr Opin Biotechnol. 2000, 11:47).

In one embodiment, secondary assays can be used to confirm that the modulating agent effects a PRMT molecule or an NFAT molecule in a NIP45 related manner. For example, compounds identified in a primary screening assay can be used in a secondary screening assay to determine whether the compound affects a NIP45-related activity.

Exemplary cell based and cell free assays of the invention are described in more detail below.

A. Cell Based Assays

The indicator compositions of the invention may be cells that express a NIP45 or a NIP45-interacting molecule (e.g., a PRMT or an NFAT protein). For example, a cell that naturally expresses endogenous polypeptide, or, more preferably, a cell that has been engineered to express one or more exogenous polypeptides, e.g., by introducing into the cell an expression vector encoding the protein may be used in a cell based assay.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell (e.g., a murine cell, or a human cell). In a preferred embodiment, the cell is a human cell.

Preferably a cell line is used which expresses low levels of endogenous NIP45 and/or NIP45-interacting polypeptide and is then engineered to express recombinant protein.

Preferably, a cell is capable of producing IFN-γ or IL-4 (either naturally or upon expression of transgenic sequences). For example, IFN-γ is naturally secreted by CD4⁺ T cells, CD 8⁺ T cells, natural killer cells and dendritic cells, while IL-4 is naturally produced by CD4⁺ T cells, and mast cells.

Recombinant expression vectors that may be used for expression of polypeptides are known in the art. For example, the cDNA is first introduced into a recombinant expression vector using standard molecular biology techniques. A cDNA can be obtained, for example, by amplification using the polymerase chain reaction (PCR) or by screening an appropriate cDNA library.

Following isolation or amplification of a cDNA molecule encoding the gene of interest, e.g., NIP45 or a NIP45-interacting polypeptide, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they air operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule in a form suitable for expression of the nucleic acid in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory-sequences) or those which direct expression of the nucleotide sequence only under certain conditions (e.g., inducible regulatory sequences).

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:134-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA may be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from mat encoding NIP45 or a NIP45-interacting polypeptide, e.g.

PRMT or, more preferably, on the same vector. Cells stably transfected with the introduced nucleic acid can be identified by drug selection e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, may be used). Use of a recombinant expression vector that allows for constitutive expression of the genes in the indicator cell is preferred for identification of compounds that enhance or inhibit the activity of the molecule. In an alternative embodiment, within the expression vector the coding sequences are operatively linked to regulatory sequences of the endogenous gene (i.e., the promoter regulatory region derived from the endogenous gene). Use of a recombinant expression vector in which expression is controlled by the endogenous regulatory sequences is preferred for identification of compounds that enhance or inhibit the transcriptional, expression of the molecule.

For example, an indicator cell can be transfected with ah expression vector comprising a polypeptide arginine methyltransferase (PRMT), incubated in the presence and in the absence of a test compound, and the effect of the compound on the expression of the molecule or on a biological response regulated by PRMT, e.g., a NIP45-related activity of PRMT1, can be determined. The biological activities of PRMT include activities determined in vivo, or in vitro, according to standard techniques. Activity can be a direct activity, such as an association with or enzymatic activity on a target molecule (e.g., a protein such as the NIP45 protein). Alternatively, activity may be an indirect activity, such as, for example, a cellular signaling activity occurring downstream of the interaction of the protein with a target molecule or a biological effect occurring as a result of the signaling cascade triggered by that interaction. For example, indirect biological activities of PRMT described herein include: the ability to modulate cytokine production, for example, T cell receptor (TCR) initiated cytokine production or non-T cell cytokine production.

Compounds that modulate cytokine production, expression and/or activity may be identified using various “read-outs”. For example, a variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

For example, in one embodiment, gene expression of NIP45, or a NIP45-binding molecule can be measured. In another embodiment, expression of a gene controlled by NIP45 (e.g., IFN-γ, IL-4, Egr2, Egr3, c-Rel, or p65) can be measured.

To determine whether a test compound modulates expression, in vitro transcriptional assays can be performed. For example, mRNA or protein expression can be measured using methods well known in the art. For instance, one or more of Northern blotting, slot blotting, ribonuclease protection, quantitative RT-PCR, or microarray analysis (e.g., Current Protocols in Molecular Biology (1994) Ausubel F M et al., eds., John Wiley & Sons, Inc.; Freeman W M et al, Biotechniques 1999 26:112; Kallioniemi et al. 2001 Ann. Med. 33:142; Blohm and Guiseppi-Eli 2001 Curr Opin Biotechnol. 12:41) may be used to confirm that expression is modulated in cells treated with a modulating agent.

In another example, agents that modulate the expression of a PRMT can be identified by operably linking the upstream regulatory sequences (e.g., the full length promoter and enhancer) of a PRMT to a reporter gene such as chloramphenicol acetyltransferase (CAT) or luciferase and introducing in into host cells. The ability of an agent to modulate the expression of the reporter gene product as compared to control cells (e.g., not exposed to the compound) can be measured.

In another exemplary embodiment, the ability of a compound to modulate the ability of PRMT and NIP45 to control cytokine gene expression can be measured. For example, a cell comprising NIP45, or a biologically active fragment thereof (e.g., amino acids 1-32 of NIP45), PRMT, and upstream regulatory sequences (e.g., the full length promoter and enhancer) of a cytokine gene, such as IFNγ or IL-4, operably linked to a reporter gene can be used to assay for the ability of a compound to modulate cytokine gene production. In one embodiment the assays are performed in the presence of NIP45 and at least one NIP 45-binding molecule, e.g., PRMT. The assay may further comprise a gene encoding at least one activator of cytokine gene transcription, e.g., T-bet or an NFAT polypeptide, e.g., NFATc1, NFATc2. In another embodiment, the assay may further comprise a maf gene. In an exemplary embodiment, the assay comprises expression vector(s) encoding PRMT, NIP45, NFATc2 and c-maf.

Specific regulatory regions of the assay components can be known in the art or can be identified by one of skill in the art utilizing, for example, DNaseI hypersensitivity mapping and/or generation of a deletion series of mutants operably linked to a reporter gene. In one embodiment, the regulatory region of a cytokine gene, e.g., IL-4, comprises a maf response element (MARE). MARE sequences are generally 13 or 14 bp elements which contain a core TRE (T-MARE) or CRE (C-MARE) palindrome respectively. MARE sequences are found in the regulatory regions of cytokine genes e.g., the IL-4 gene. Non-limiting examples of regulatory regions of the IL-4 gene that can be utilized include, for example, about 3 kb of the upstream regulatory region of the IL-4 gene, nucleotide positions −157 to +58, −42 to −37, −59 to −28, relative to the start of transcription of the IL-4 gene, e.g., +1 (see, e.g., Hodge, M., et al. (1995) J. Immunol. 154:6397 and Ho, I. C., et. al. (1996) Cell 85:973), and −732 to +68, relative to the start of transcription of the IL-4 gene (see Examples 6 and 8). In another embodiment, the regulatory region of the IFNγ gene is operably linked to a reporter gene and is used to assay for the ability of a compound to modulate cytokine gene production. In one embodiment, a 9.2 kb IFNγ luciferase reporter construct is utilized (see, e.g., Example 10 and Szabo, S. J., et al. (2000) Cell 100:655, the contents of which are expressly incorporated herein by reference). Other non-limiting examples of regulatory regions of the IFNγ gene that can be utilized include, for example, −108 to −40, −70 to −47, −98 to −72, −251 to −214, and −565 to +64 relative to the start of transcription of the IFNγ gene (see, e.g., Aune, T. M., et al. (1997) Mol. Cell. Biol. 17:199; Penix, L., et al. (1993) J. Exp. Med. 178:1483; and Soutto, M., et al. (2002) J. Immunol. 169:4205). Alternatively, a genomic fragment containing the IFNγ gene as well as regulatory elements upstream and/or intragenically operably linked to a reporter gene can be utilized in the subject screening assays (see, e.g., Soutto, M., et al. (2002) J. Immunol. 169:6664). A change in reporter gene expression (e.g., as compared to a control not exposed to the compound) identifies the compound as a modulator of cytokine gene expression. Other techniques are well known to those of skill in the art. Additional exemplary techniques are illustrated in the instant examples.

As used interchangeably herein, the terms “operably linked” and “operatively linked” are intended to mean that the nucleotide sequence is linked to a regulatory sequence in a manner which allows expression of the nucleotide sequence in a host cell (of by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

In one embodiment, the level of expression of the reporter gene in the indicator cell in the presence of the test compound is higher than the level of expression of the reporter gene in the indicator cell in the absence of the test compound and the test compound is identified as a compound that stimulates the expression of a cytokine gene. In another embodiment, the level of expression of the reporter gene in the indicator cell in the presence of the test compound is lower than the level of expression of the reporter gene in the indicator cell in the absence of the test compound and the test compound is identified as a compound that inhibits the expression of a cytokine gene.

In another embodiment, protein expression may be measured. For example, standard techniques such as Western blotting or in situ detection can be used.

In one embodiment, the ability of a compound to modulate cytokine production, can be determined by measuring the intracellular concentration of a cytokine (using intracellular cytokine FACS). In one embodiment, the ability of a compound to modulate cytokine production can be determined by measuring the concentration of the cytokine secreted by a cell. For example, IFNγ or can be measured by measuring the effect of the supernatant on an indicator cell line (e.g., on proliferation of the indicator cell line), or, e.g., in an ELISA assay.

In one embodiment a downstream effect of modulation of cytokine production, e.g., the effect of a compound on differentiation of cells, e.g., T cells, may be used as an indicator of modulation of NIP45 or a NIP45-interacting protein. Cell differentiation can be monitored directly (e.g. by microscopic examination of the cells for monitoring cell differentiation), or indirectly, e.g., by monitoring one or more markers of cell differentiation (e.g., an increase in mRNA for a gene product associated with cell differentiation, or the secretion of a gene product associated with cell differentiation, such as the secretion of a protein (e.g., the secretion of cytokines) or the expression of a cell surface marker. Standard methods for detecting mRNA of interest, such as reverse transcription-polymerase chain reaction (RT-PCR) and Northern blotting, are known in the art. Standard methods for detecting protein secretion in culture supernatants, such as enzyme linked immunosorbent assays (ELISA), are also known in the art. Proteins can also be detected using antibodies, e.g., in an immunoprecipitation reaction, or by staining and FACS analysis.

In another embodiment, the ability of a compound to modulate immune cell function (e.g., effector T cell function) can be determined. For example, in one embodiment, the ability of a compound to modulate cellular function(s) dependent on exposure to cytokines. For example, cell proliferation, cell differentiation, cytokine production, cytotoxicity, or phagocytosis can be measured using techniques well known in the art. A number of art-recognized readouts can be used.

The ability of the test compound to modulate NIP45 or a NIP45-interacting polypeptide binding to a substrate or target molecule can also be determined. Determining the ability of the test compound to modulate, for example, PRMT, binding to a target molecule (e.g., a binding partner such as a substrate) can be accomplished, for example, by determining the ability of the molecules to be coimmunoprecipitated or by coupling the target molecule with a radioisotope or enzymatic label such that binding of the target molecule to NIP45 or a NIP45-interacting polypeptide can be determined, e.g., by detecting the labeled NIP45 target molecule in a complex. Alternatively, for example, PRMT, can be coupled with a radioisotope or enzymatic label to monitor the ability of a test compound to modulate, PRMT, binding to a target molecule in a complex.

Whereas the ability of NIP45 to bind to PRMT or NFAT is associated with increased cytokine production, the ability of NIP45 to bind to TRAF2 is associated with decreased binding of NIP45 to NFAT and decreased IL-4 production (Lieberson et al. 2001 J. Exp. Med. 194:89). Accordingly, in another embodiment, the ability of a compound to modulate the binding of NIP45 to TRAF2 can also be measured. Increased binding to TRAF2 is associated with decreased NIP45 activity and decreased cytokine production.

Determining the ability of the test compound to bind to NIP45 can be accomplished, for example, by coupling the compound with a radioisotope or enzymatic label such that binding of the compound can be determined by detecting the labeled compound in a complex. For example, (targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

In another embodiment, fluorescence technologies can be used, e.g., fluorescence polarization, time-resolved fluorescence, and fluorescence resonance energy transfer (Selvin P R, Nat. Struct. Biol. 2000 7:730; Hertzberg R P and Pope A J, Crurr Opin Chem Biol. 2000 4:445).

It is also within the scope of this invention to determine the ability of a compound to interact with NIP45, a NIP45-interacting molecule without the labeling of any of the interactants. For example, a microphysiometer may be used to detect the interaction of a compound with a NIP45, a NIP45-interacting molecule without the labeling of either the compound or the molecule (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate may be used as an indicator of the interaction between compounds.

In yet another aspect of the invention, the NIP45 or a NIP45-interacting polypeptide protein or fragments thereof may be used as “bait protein” e.g., in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with NIP45or a NIP45-interacting polypeptide (“binding proteins” or “bp”) and are involved in NIP45 or a NIP45-interacting molecule activity. Such NIP45- or NIP45-interacting molecule-binding proteins are also likely to be involved in the propagation of signals by the NIP45 or a NIP45-interacting molecule proteins. The two-hybrid system is based on the modular nature of most transcription factors, which consist of separable DNA-binding and activation domains. Briefly, the assay utilizes two different DNA constructs. In one construct, the gene that codes for a NIP45 or a NIP45-interacting molecule protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the bait and the “prey” proteins are able to interact, in vivo, forming an NIP45- or a NIP45-interacting molecule-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the NIP45 or a NIP45-interacting molecule protein.

B. Cell-Free Assays

Alternatively, the indicator composition can be a cell-free composition that includes a NIP45 and/or a NIP45-interacting molecule (e.g., PRMT1 or an NFAT protein) e.g., a cell extract from a cell expressing the protein or a composition that includes purified either natural or recombinant protein.

In one embodiment, the indicator composition is a cell free composition. Polypeptides expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies may be used to produce a purified or semi-purified protein that may be used in a cell free composition. Alternatively, a lysate or an extract of cells expressing the protein of interest can be prepared for use as cell-free composition. Cell extracts with the appropriate post-translation modifications of proteins can be prepared using commercially available resources found at, for example Promega, Inc., and include but are not limited to reticulocyte lysate, wheat germ extract and E. coli S30 extract.

In one embodiment, compounds that specifically modulate an activity of NIP45 or a NIP45-binding molecule may be identified. For example. Compounds that modulate an activity of PRMT (e.g., a NIP45 related activity) are identified based on their ability to modulate the interaction of PRMT with a target molecule to which PRMT binds, e.g., NIP45. In another embodiment, compounds that modulate an activity of NIP45 are identified based on their ability to modulate interaction of NIP45 with a NIP45-binding molecule, e.g., NFAT. Suitable assays are known in the art that allow for the detection of protein-protein interactions (e.g., immunoprecipitations and the like) or that allow for the detection of interactions between a DNA binding protein with a target DNA sequence (e.g., electrophoretic mobility shift assays, DNAse I footprinting assays and the like). By performing such assays in the presence and absence of test compounds, these assays may be used to identify compounds that modulate (e.g., inhibit or enhance) the interaction of NIP45 or a NIP45-binding molecule with a target molecule.

In the methods of the invention for identifying test compounds that modulate an interaction between a NIP45-interacting protein and NIP45, the complete NIP45 protein may be used in the method, or, alternatively, only portions of the protein may be used. For example, an isolated NIP45 domain (e.g., consisting of amino acids 1-32 or a larger subregion comprising amino acids 1-32 or a fusion protein comprising amino acids 1-32 of NIP45) may be used. An assay may be used to identify test compounds that either stimulate or inhibit the interaction between the NIP45 protein and a target molecule. A test compound that stimulates the interaction between the protein and a target molecule is identified based upon its ability to increase the degree of interaction between (e.g., NIP45 and a target molecule or PRMT and a target molecule) as compared to the degree of interaction in the absence of the test compound and such a compound would be expected to increase the activity of NIP45 in the cell. A test compound that inhibits the interaction between the protein and a target molecule is identified based upon its ability to decrease the degree of interaction between the protein and a target molecule as compared to the degree of interaction in the absence of the compound and such a compound would be expected to decrease NIP45 activity.

In one embodiment, the amount of binding of NIP45 to a NIP45-interacting molecule in the presence of the test compound is greater than the amount of binding in the absence of the test compound, in which case the test compound is identified as a compound that enhances binding of NIP45 to a NIP45 interacting molecule In another embodiment, the amount of binding of the NIP45 to the binding molecule in the presence of the test compound is less than the amount of binding of NIP45 to the binding molecule in the absence of the test compound, in which case the test compound is identified as a compound that inhibits binding of NIP45 to the binding molecule.

For example, binding of the test compound to NIP45 or a NIP45-interacting polypeptide can be determined either directly or indirectly as described above. Determining the ability of NIP45 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) may be used as an indication of real-time reactions between biological molecules.

In another embodiment, the ability of a compound to modulate the ability of NIP45 or a NIP45-interacting molecule to be acted on by an enzyme or to act on a substrate can be measured. In one embodiment, transferase assays can be used to detect the ability of PRMTs to methylate at least one arginine residue on a substrate. Such assays are well known in the art and may be performed as described (e.g., Tang et al. 2000 J. Biol. Chem. 275:7723). In another embodiment, cell lysates may be harvested and immunoprecipitated with antibodies to isolate individual polypeptides for analysis of arginine methylation. Immunoprecipitates (e.g., of NIP45) may then be subjected to an in vitro methylation assay, e.g., using recombinant PRMT1.

For example, in one embodiment, hypomethylated cell lysates can produced and the ability of PRMTs to methylate various substrates after addition of 3H—S-adenosylmethionene is evaluated. In another embodiment, an anti-methylated arginine antibody (e.g., specific for asymmetrically methylated arginines within RG repeats similar to those found in the amino-terminus of NIP45) can be used to identify polypeptides containing methylated arginine residues.

In one embodiment of the above assay methods, it may be desirable to immobilize either NIP45 or a NIP45-interacting polypeptide for example, to facilitate separation of complexed from uncomplexed forms of one or both of the proteins, or to accommodate automation of the assay. Binding to a surface can be accomplished in any vessel suitable for containing the reactants. Examples of such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or NIP45 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, proteins may be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates Pierce Chemical). Alternatively, antibodies which are reactive with protein or target molecules but which do not interfere with binding of the protein to its target molecule can be derivatized to the wells of the plate, and unbound target or NIP45 protein is trapped in the wells by antibody conjugation. Methods for detecting such complexes, in addition to those described above for the GST-immobilized complexes, include immunodetection of complexes using antibodies reactive with NIP45 or a NIP45-interacting polypeptide or target molecule, as well as enzyme-linked assays which rely on detecting an enzymatic activity associated with the NIP45 protein or binding molecule.

C. Assays Using Knock-Out Cells

In another embodiment, the invention provides methods for identifying compounds that modulate a cytokine production in cells deficient in NIP45. As described in the Examples, inhibition of NIP45 activity (e.g., by disruption of the NIP45 gene) in immune cells results, e.g., in a deficiency of IL-4 and IFN-γ production. Thus, cells deficient in NIP45 and/or a NIP45-binding molecule (e.g., PRMT or NFAT) may be used to identify agents that modulate a biological response regulated by NIP45 by means other than modulating NIP45 itself (i.e., compounds that “rescue” the NIP45 deficient phenotype). Alternatively, a “conditional knock-out” system, in which the gene is rendered non-functional in a conditional manner, may be used to create deficient cells for use in screening assays. For example, a tetracycline-regulated system for conditional disruption of a gene as described in WO 94/29442 and U.S. Pat. No. 5,650,298 may be used to create cells, or animals from which cells can be isolated, deficient in specific polypeptides in a controlled manner through modulation of the tetracycline concentration in contact with the cells. Specific cell types, e.g., lymphoid cells (e.g., thymic, splenic and/or lymph node cells) or purified cells such as T cells from such animals may be used in screening assays. In one embodiment, the entire 5.4 kB exon 2 of NIP45 can be replaced, e.g., with a neomycin cassette, resulting in an allele that produces no NIP45 protein. This embodiment is described in the appended examples.

In the screening method, cells deficient in NIP45 or a NIP45-binding molecule can be contacted with a test compound and a biological response regulated by NIP45 measured. Modulation of the response in the cells deficient in NIP45 (as compared to an appropriate control such as, for example, untreated cells or cells treated with a control agent) identifies a test compound as a modulator of the response.

In one embodiment, the test compound is administered directly to a non-human knock out animal, preferably a mouse (e.g., a mouse in which the NIP45 gene or NIP45 interacting molecule gene, such as a PRMT gene) is conditionally disrupted by means described above, or a chimeric mouse in which the lymphoid organs are deficient in the gene, to identify a test compound that modulates the in vivo responses of cells deficient in the gene. In another embodiment, cells deficient in the gene are isolated from the non-human animal and contacted with the test compound ex vivo to identify a test compound that modulates a response regulated by the gene in the cells

Preferred non-human animals include monkeys, dogs, cats, mice, rats, cows, horses, goats and sheep. In preferred embodiments, the deficient animal is a mouse. Mice deficient in the gene ban be made using methods known in the art. One example of such a method and the resulting NIP45 heterozygous and homozygous animals is described in the appended examples. Non-human animals deficient in a particular gene product typically are created by homologous recombination. In an exemplary embodiment, a vector is prepared which contains at least a portion of the gene into which a deletion, addition or substitution has been introduced to thereby alter, e.g., functionally disrupt, the endogenous gene. The gene preferably is a mouse gene. For example, a mouse gene can be isolated from a mouse genomic DNA library using the mouse cDNA as a probe. The mouse gene then may be used to construct a homologous recombination vector suitable for modulating an endogenous gene in the mouse genome. In a preferred embodiment, the vector is designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced gene has homologously recombined with the endogenous gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells may be used to breed animals in which all cells of the animal contain the homologously recombined DNA by germline transmission of the transgene. Methods for constructing homologous recombination vectors and homologous recombinant animals are: described further in Bradley, A. (1991) Current Opinion in Biotechnology 2:823-829 and in PCT International Publication Nos.: WO 90/11354 by LeMouellec et al.; WO 91/01140 by Smithies et al.; WO 92/0968 Zijlstra et al.; and WO 93/04169 by Berns et al.

In one embodiment, compounds that modulate cytokine production are identified by contacting cells deficient in one or more test compounds ex vivo with the compound and determining the effect of the test compound on a read-out. In one embodiment, deficient cells contacted with a test compound ex vivo can be readministered to a subject.

In one embodiment of the screening assay, compounds tested for their ability to modulate cytokine production are contacted with deficient cells by administering the test compound to a non-human deficient animal in vivo and evaluating the effect of the test compound on the response in the animal.

The test compound can be administered to a non-knock out animal as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions. Pharmaceutical compositions are described in more detail below.

For practicing the screening method ex vivo, cells deficient, e.g., in NIP45, can be isolated from a non-human deficient animal or embryo by standard methods and incubated (i.e., cultured) in vitro with a test compound. Cells (e.g., T cells) can be isolated from e.g., NIP45, deficient animals by standard techniques.

Following contact of the deficient cells with a test compound (either ex vivo or in vivo), the effect of the test compound on cytokine can be determined by any one of a variety of suitable methods, such as those set forth herein, e.g., including light microscopic analysis of the cells, histochemical analysis of the cells, production of proteins, induction of certain genes, e.g., cytokine genes, such as IL-4 and IFN-γ.

### III. TEST COMPOUNDS

A variety of test compounds can be evaluated using the screening assays described herein. The term “test compound” includes any reagent or test agent which is employed in the assays of the invention and assayed for its ability to influence the production, expression and/or activity of cytokines. More than one compound, e.g., a plurality of compounds, can be tested at the same time for their ability to modulate cytokine production, expression and/or activity in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening may be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68-; and in Gallop et al. (1994); J. Med. Chem. 37:1233.

Exemplary compounds which can be screened for activity include, but are not limited to, peptides, nucleic acids, carbohydrates, small organic, molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., antibodies (e.g., intracellular, polyclonal, monoclonal, humanized, anti-idiotypic, chimeric, and single chain antibodies as well as Fab, F(ab′)², Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of molecules (e.g., dominant negative mutant forms of NIP45 or a NIP45-binding protein).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques. 13:412-421), or on beads, (Lam (1991) Nature 354:82-84), chips (Fodor (1993) Nature 364:555-556), bacteria (Ladner U.S. Pat. No. 5,223,409); spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Compounds identified in the subject screening assays may be used, e.g., in methods of modulating receptor mediated signaling, cytokine production or in methods of modulating one or more of the biological responses regulated by cytokine expression, and/or activity, e.g., the modulation of immune cell effector function, or the relative number of Th1/Th2 cells. It will be understood that it may be desirable to formulate such compound(s) as pharmaceutical compositions (described supra) prior to contacting them with cells.

Once a test compound is identified that directly or indirectly modulates, e.g., production, expression and/or activity of a gene regulated by NIP45 and/or a NIP45-binding molecule, by one of the variety of methods described herein, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

The instant invention also pertains to compounds identified in the subject screening assays.

### IV. PHARMACEUTICAL COMPOSITIONS

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion, medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### V. METHODS OF USE

The present invention provides for both prophylactic and therapeutic methods of treating a subject at risk of (or susceptible to) a disorder or having a disorder that would benefit from modulation of NIP45 or a NIP45-binding molecule. Such disorders include those associated with an aberrant cytokine production, e.g., inappropriate Th1 or Th2 responses. For example, an immune system disorder or condition associated with an undesirable immune response (such as an unwanted or excessive inflammatory response, an autoimmune disorder, graft-versus-host disease (GVHD), an allogeneic transplant) or an immune system disorder or condition that would benefit from an enhanced immune response, e.g. immunosuppression.

In one aspect, the invention provides a method for preventing in a subject, a disease or condition associated with an aberrant or unwanted immune response or, alternatively, an abnormally low immune response, by administering to the subject an agent which modulates the activity of NIP45 or a NIP45-interacting polypeptide, e.g., PRMT1. Subjects at risk for such disorders can be identified, for example, using methods described herein or any one or a combination of diagnostic or prognostic assays known in the art. Administration of a prophylactic agent can occur prior to the manifestation of symptoms characteristic of the aberrant immune response, such that a disease or disorder is prevented or, alternatively, delayed in its progression. Depending on the type of immune response aberrancy, for example, a compound that stimulates the expression and/or activity of NIP45 or a NIP45 biding molecule or a compound that inhibits the expression and/or activity of NIP45 or a NIP45-binding molecule may be used for treating a subject. Agents for use can be known (e.g., sense or antisense nucleic acid molecules encoding NIP45 or NIP45 interacting molecules or the polypeptides they encode) or can be identified, e.g., using the screening assays described herein (e.g., a PRMT1 agonist or antagonist, a peptidomimetic of a PRMT1 agonist or antagonist, a PRMT1 peptidomimetic, or other small molecule).

Modulatory methods of the invention involve contacting a cell (e.g., an immune cell, e.g., a T cell or a non-T cell) with a agent that modulates the activity and/or expression of NIP45 and/or a NIP45 interacting molecule.

These modulatory methods can be performed in vitro (e.g., by contacting the cell with the agent) or, alternatively, in vivo (e.g., by administering the agent to a subject). As such, the present invention provides methods of treating an individual afflicted with a condition or disorder that would benefit from up- or down-modulation of cytokine production, e.g., a disorder characterized by an unwanted, insufficient, or aberrant immune response. In one embodiment, the method involves administering an agent (e.g., an agent identified by a screening assay described herein), or combination of agents that modulates (e.g., upregulates or downregulates) cytokine expression and/or activity.

Inhibition of cytokine production is desirable in situations in which cytokine production is abnormally upregulated and/or in which decreased cytokine production is likely to have a beneficial effect, for example in a situation of an excessive or unwanted immune response. Such situations include conditions, disorders, or diseases such as an autoimmune disorder, a transplant (e.g., a bone marrow transplant, a stem cell transplant, a heart transplant, a lung transplant, a liver transplant, a kidney transplant, a cornea transplant, or a skin transplant), graft versus host disease (GVHD), an allergy, or in inflammatory disorder. Likewise, upregulation of cytokine production is desirable in situations in which cytokine production is abnormally downregulated and/or in which increased cytokine production is likely to have a beneficial effect (e.g., in a neoplasia). In addition, INF-γ production is not detected in AIDS patients and has been shown to be important for controlling infection by intracellular parasites, e.g., Cryptosporidium, and preventing chronic disease (White, et al. (2000) J. Infect. Dis. 181: 701-709). In addition, Takayanagi, H., et al. ((2000) Nature 408:600-605) demonstrated that T-cell production of INF-γ strongly suppresses osteoclastogenesis. Further, enhance immune responses may be of benefit in treating neoplastic conditions.

As used herein, the term “autoimmunity” refers to the condition in which a subject's immune system starts reacting against his or her own tissues. Non-limiting examples of autoimmune diseases and disorders having an autoimmune component that would benefit from modulation of a cytokine production include type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis.

The terms “neoplasia,” “hyperplasia,” and “tumor” are often commonly referred to as “cancer,” which is a general name for more than 100 disease that are characterized by uncontrolled, abnormal growth of cells. Examples of malignancies include but are not limited to acute lymphoblastic leukemia; acute myeloid leukemia; adrenocortical carcinoma; AIDS-related lymphoma; cancer of the bile duct; bladder cancer; bone cancer, osteosarcomal malignant fibrous histiocytomal brain stem gliomal brain tumor; breast cancer, bronchial adenomas; carcinoid tumors; adrenocortical carcinoma; central nervous system lymphoma; cancer of the sinus, cancer of the gall bladder; gastric cancer; cancer of the salivary glands; cancer of the esophagus; neural cell cancer; intestinal cancer (e.g., of the large or small intestine); cervical cancer; colon cancer, colorectal cancer; cutaneous T-cell lymphoma; B-cell lymphoma; T-cell lymphoma; endometrial cancer; epithelial cancer; endometrial cancer; intraocular melanoma; retinoblastoma; hairy cell leukemia; liver cancer; Hodgkin's disease; Kaposi's sarcoma; acute lymphoblastic leukemia; lung cancer; non-Hodgkin's lymphoma; melanoma; multiple myeloma; neuroblastoma; prostate cancer; retinoblastoma; Ewing's sarcoma; vaginal cancer; Waldenstrom's macroglobulinemia; adenocarcinomas; ovarian cancer, chronic lymphocytic leukemia, pancreatic cancer, and Wilm's tumor.

Exemplary agents for use in upmodulating cytokine production include, e.g., nucleic acid molecules encoding NIP45 or NIP45-interacting molecule polypeptides (e.g., PRMT or NFAT), NIP45 or NIP45-interacting molecule polypeptides, and compounds that stimulate the expression and/or activity of NIP45 or a NIP45-binding molecule interaction of NIP45 with PRMT1 or NFAT family members).

Exemplary agents for use in downmodulating cytokine production (i.e., cytokine antagonists) include agents that inhibit the activity of NIP45 or NIP45-interacting molecule in an immune cell (e.g., antisense molecules, siRNA molecules, dominant negative mutants, or compounds identified in the subject screening assays).

A. Downregulation of Immune Responses

There are numerous embodiments of the invention for downregulating the function of a cytokines to thereby downregulate immune responses. Downregulation can be in the form of inhibiting or blocking an immune response already in progress, or may involve preventing the induction of an immune response. The functions of activated immune cells can be inhibited by downregulating immune cell responses or by inducing specific anergy in immune cells, or both.

For example, cytokine activity can be inhibited by contacting a cell which expresses NIP45 or NIP45-interacting molecule with an agent that inhibits the expression and/or activity NIP45 or NIP45-interacting molecule.

In another embodiment, immune responses can be downregulated in a subject by removing immune cells from the patient, contacting the immune cells in vitro with an agent (e.g., a small molecule) that downregulates NIP45 or NIP45-interacting molecule activity, and reintroducing the in vitro-stimulated immune cells into the patient.

Downregulating immune responses by inhibiting NIP45 activity or a NIP45-interacting polypeptide activity is useful in downmodulating the immune response, e.g., in situations of tissue, skin and organ transplantation, in graft-versus-host disease (GVHD), or allergies, or in autoimmune diseases such as systemic lupus erythematosus and multiple sclerosis. For example, blockage of Th1 cytokine production may result in reduced tissue destruction in tissue transplantation. Typically, in tissue transplants, rejection of the transplant is initiated through its recognition as foreign by immune cells, followed by an immune reaction that destroys the transplant. The administration of a molecule which inhibits the activity of NIP45, e.g., by blocking the interaction of NIP45 with, for example, NFAT family members or PRMT, in immune cells alone or in conjunction with another downmodulatory agent can inhibit the generation of an immune response.

An immune response can be further inhibited by the use of an additional agent that can downmodulate the immune response, as described further herein. Downmodulatory agents that may be used in connection with the downmodulatory methods of the invention include, for example, blocking antibodies against other immune cell markers, or soluble forms of other receptor ligand pairs (e.g., agents that disrupt the interaction between CD40 and, CD40 ligand (e.g., anti CD40 ligand antibodies)), antibodies against cytokines, general immunosuppressive drugs (e.g., FK506, cyclosporin, rapamycin, steroids) or inhibitors of IL-4, e.g., AG-490, a Janus tyrosine kinase (JAK) 2-JAK3 inhibitor.

Inhibition of cytokine production, in particular Th2 cytokine production, may also be useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. For example, administration of agents that inhibit an activity of NIP45 or NIP45-interacting molecule may lead to long-term relief from the disease. Additionally, co-administration of agents which block costimulation of immune cells by disrupting receptor-ligand interactions may be useful in inhibiting immune cell activation to prevent production of autoantibodies or cytokines which may be involved in the disease process. The efficacy of reagents in preventing or alleviating autoimmune disorders can be determined using a number of well-characterized animal models of human autoimmune diseases. Examples include murine experimental autoimmune encephalitis, systemic lupus erythematosus in MBL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Inhibition of Th2 cytokine production is also useful therapeutically in the treatment of allergies and allergic reactions, e.g., by inhibiting IgE production. An agent that inhibits, for example, NIP45 or NIP45-interacting molecule activity can be administered to an allergic subject to inhibit immune cell-mediated allergic responses in the subject. Inhibition of NIP45 or NIP45-interacting molecule activity can be accompanied by exposure to allergen in conjunction with appropriate MHC molecules. Allergic, reactions can, be systemic, or local in nature, depending on the route of entry of the allergen and the pattern of deposition of IgE on mast cells or basophils. Thus, immune cell-mediated allergic responses can be inhibited locally or systemically by administration of an agent that inhibits NIP45 or NIP45-interacting molecule activity.

Downregulation of immune cell activation through inhibition of cytokine production may also be important therapeutically in pathogenic infections of immune cells (e.g., by viruses or bacteria). For example, in the acquired immune deficiency syndrome (AIDS), viral replication is stimulated by immune cell activation. Inhibition of NIP45 or NIP45-interacting molecule activity may result in inhibition of viral replication and thereby ameliorate the course of AIDS.

Downregulation of immune cell activation via inhibition of cytokine production may also be useful in treating inflammatory disorders and in promoting the maintenance of pregnancy when there exists a risk of immune-mediated spontaneous abortion.

i. Exemplary Inhibitory Compounds

Since inhibition of cytokine production is associated with an decreased immune response, to downmodulate or inhibit the immune response, cells (e.g., T cells) are contacted with an agent that inhibits NIP45 or a NIP45-interacting molecule activity. The immune cells may be contacted with the agent in vitro and then the cells can be administered to a subject or, alternatively, the agent may be administered to the subject (e.g., directly to an articular site at which T growth and/or differentiation is desired). The methods of the invention using cytokine inhibitory compounds may be used in the treatment of disorders in which the immune response is diminished, blocked, inhibited, downregulated or the like.

Inhibitory compounds of the invention can be, for example, intracellular binding molecules that act to specifically inhibit the expression or activity of NIP45 or a NIP45-interacting molecule. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules, described in further detail below, include antisense nucleic acids, intracellular antibodies, peptidic compounds that inhibit the interaction of NIP45 or a NIP45-interacting molecule with a target molecule (e.g., calcineurin) and chemical agents that specifically inhibit NIP45 or a NIP45-interacting molecule activity.

a. Antisense Nucleic Acid Molecules

In one embodiment, an inhibitory compound of the invention is an antisense nucleic acid molecule that is complementary to a gene encoding NIP45 or a NIP45-interacting molecule, or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the coding strand sequences encoding NIP45 or a NIP45-interacting molecule disclosed herein, antisense nucleic acids of the invention can be designed according to the rules of Watson and Crick base pairing. The antisense nucleic acid molecule can be complementary to the entire coding region of NIP45 or a NIP45-interacting molecule mRNA, but more preferably is an oligonucleotide which is antisense to only a portion of the coding or noncoding region of NIP45 or a NIP45-interacting molecule mRNA. For example, the antisense oligonucleotide can be complementary to the region surrounding the translation start site of NIP45 or a NIP45-interacting molecule mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides may be used. Examples of modified nucleotides which may be used to generate the antisense nucleic acid include 5-fluorouracil, 5-bromouracil, 5-chlorouracil, 5-iodouracil, hypoxanthine, xantine, 4-acetylcytosine, 5-carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouraoil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/of genomic DNA encoding a NIP45 or a NIP45-interacting molecule protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid of the invention is a compound that mediates RNAi. RNA interfering agents include, but are not limited to, nucleic acid molecules including RNA molecules which are homologous to the target gene or genomic sequence, e.g., NIP45 or PRMT, or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion.

Non-limiting exemplary siRNA molecules specific for the murine NIP45 gene (SEQ ID NO.: 1) that can be utilized in the methods of the invention, include, for example:

Beginning at position 388:

| | |
|---|---|
| Sense strand siRNA: | |
| CCUCAUUCCAGAUAAUUCAtt | (SEQ ID NO.:11) |
| Antisense strand siRNA: | |
| UGAAUUAUCUGGAAUGAGGtt | (SEQ ID NO.:12) |

Beginning at position 653:

| | |
|---|---|
| Sense strand siRNA: | |
| GUGAACAAGCGUCUCCAAGtt | (SEQ ID NO.:13) |
| Antisense strand siRNA: | |
| CUUGGAGACGCUUGUUCACtt | (SEQ ID NO.:14) |

Beginning at position 1220:

| | |
|---|---|
| Sense strand siRNA: | |
| UCCGGAGAUCUCAUCGAAGtt | (SEQ ID NO.:15) |
| Antisense strand siRNA: | |
| CUUCGAUGAGAUCUCCGGAtt | (SEQ ID NO.:16) |

Non-limiting exemplary siRNA molecules specific for the murine PRMT1 gene (SEQ ID NO.:9) that can be utilized in the methods of the invention, include, for example:

Beginning at position 276:

| | |
|---|---|
| Sense strand siRNA: | |
| AGACAAGGUGGUGCUGGAUtt | (SEQ ID NO.:17) |
| Antisense strand siRNA: | |
| AUCCAGCACCACCUUGUCUtt | (SEQ ID NO.:18) |

Beginning at position 477:

| | |
|---|---|
| Sense strand siRNA: | |
| GGUGGACAUCAUCAUCAGCtt | (SEQ ID NO.:19) |
| Antisense strand siRNA: | |
| GCUGAUGAUGAUGUCCACCtt | (SEQ ID NO.:20) |

Beginning at position 990:

| | |
|---|---|
| Sense strand siRNA: | |
| GACUGGCGAGGAGAUCUUUtt | (SEQ ID NO.:21) |
| Antisense strand siRNA: | |
| AAAGAUCUCCUCGCCAGUCtt | (SEQ ID NO.:22) |

In one embodiment one or more of the chemistries described above for use in antisense RNA can be employed.

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) may be used to catalytically cleave NIP45 or a NIP45-interacting molecule mRNA transcripts to thereby inhibit translation of NIP45 mRNA. A ribozyme having specificity for a NIP45- or a NIP45-interacting molecule-encoding nucleic acid can be designed, e.g., based upon the nucleotide sequence of SEQ ID NO:1 or another nucleic acid molecule encoding another NIP45 or a NIP45-interacting molecule family polypeptide. For example, a derivative of a Tetrahymena L-19 IVS RNA can be constructed in which the nucleotide sequence of the active site is complementary to the nucleotide sequence to be cleaved in a NIP45- or a NIP45-interacting molecule-encoding mRNA. See, e.g., Cech et al. U.S. Pat. No. 4,987,071; and Cech et al. U.S. Pat. No. 5,116,742. Alternatively, NIP45 mRNA may be used to select a catalytic RNA having a specific ribonuclease activity from a pool of RNA molecules. See, e.g., Bartel, D. and Szostak, J. W., 1993, Science 261:1411-1418.

Alternatively, gene expression can be inhibited by targeting nucleotide sequences complementary to the regulatory region of NIP45 or a NIP45-interacting molecule (e.g., the NIP45 promoter and/or enhancers) to form triple helical structures that prevent transcription of the NIP45 gene in target cells. See generally, Helene, C., 1991, Anticancer Drug Des. 6(6):569-84; Helene, C. et al., 1992, Ann N.Y. Acad. Sci. 660:27-36; and Maher, L. J., 1992, Bioassays 14(12):807-15.

In yet another embodiment, the NIP45 or a NIP45-interacting molecule nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified regenerate peptide nucleic acids (see Hyrup B. et al, 1996, Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al., 1996, supra; Perry-O'Keefe et al., 1996, Proc. Natl. Acad. Sci. USA 93: 14670-675.

PNAs of NIP45 or a NIP45-interacting molecule nucleic acid molecules may be used in therapeutic and diagnostic applications. For example, PNAs may be used as antisense or antigene agents for sequence-specific modulation of gene expression by, for example, inducing transcription or translation arrest or inhibiting replication. PNAs of NIP45 or a NIP45-interacting molecule nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., 1996, supra)); or as probes or primers for DNA sequencing or hybridization (Hyrup B. et al., 1996, supra; Perry-O'Keefe supra).

In another embodiment, PNAs of NIP45 or a NIP45-interacting molecule can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of NIP45 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B., 1996, supra). The synthesis of PNA-DNA chimeras can be performed as described in Hyrup B., 1996, supra and Finn P. J. et al., 1996, Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, may be used as a between the PNA and the 5′ end of DNA (Mag, M. et al., 1989, Nucleic Acid Res. 17:5973-88). PNA monomers are then coupled in a stepwise manner to produce a chimeric molecule with a 5′ PNA segment and a 3′ DNA segment (Finn P. J. et al., 1996, supra). Alternatively, chimeric molecules can be synthesized with a 5′ DNA segment and a 3′ PNA segment (Peterser, K. H. et al., 1975, Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. US. 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., 1988, Bio-Techniques 6:958-976) or intercalating agents. (See, e.g., Zon, 1988, Pharm. Res. 5:539-549). To this end, the oligonucleotide may be conjugated to another molecule, (e.g., a peptide, hybridization triggered cross-linking agent, transport agent, or hybridization-triggered cleavage agent).

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA-species.

b. Intracellular Antibodies

Another type of inhibitory compound that may be used to inhibit the expression and/or activity of cytokine production in a cell is an intracellular antibody specific for NIP45 or a NIP45-interacting molecule discussed herein. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993). Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 262:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of transcription factor activity according to the inhibitory methods of the invention, preferably an intracellular antibody that specifically binds the transcription factor is expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N- or C-terminus of the light and heavy chain genes (see e.g., Biocca, S. et al. (1990) EMBO J. 9:101-108; Mhashilkar, A. M. et al. (1995) EMBO J. 14:1542-1551). A preferred nuclear localization signal to be used for nuclear targeting of the intracellular antibody chains is the nuclear localization signal of SV40 Large T antigen (see Biocca, S. et al. (1990) EMBO J. 9:101-108, Mhashilkar, A. M. et al. (1995) EMBO J. 14:1542-1551).

To prepare an intracellular antibody expression vector, antibody light and heavy chain cDNAs encoding antibody chains specific for the target protein of interest, e.g., NIP45 or a NIP45-interacting molecule protein, is isolated, typically from a hybridoma that secretes a monoclonal antibody specific for NIP45 or a NIP45-interacting molecule protein. For example, antibodies can be prepared by immunizing a suitable subject, (e.g., rabbit, goat, mouse or other mammal) with a NIP45 or a NIP45-interacting molecule protein immunogen. An appropriate immunogenic preparation can contain, for example, recombinantly expressed NIP45 or a NIP45-interacting molecule protein or a chemically synthesized NIP45 or a NIP45-interacting molecule peptide. The preparation can further include an adjuvant, such as Freund's complete or incomplete adjuvant, or similar immunostimulatory compound. Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol. 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet, 3:231-36). Briefly, an immortal cell line (typically a myeloma) is fused to lymphocytes (typically splenocytes) from a mammal immunized with a NIP45 protein immunogen as described above, and the culture supernatants of the resulting hybridoma cells are screened to identify a hybridoma producing a monoclonal antibody that binds specifically to the NIP45 or a NIP45-interacting molecule protein. Any of the many well known protocols used for fusing lymphocytes and immortalized cell lines can be applied for the purpose of generating an anti-NIP45 protein monoclonal antibody (see, e.g., G. Galfre et al. (1977) Nature 266:550-52; Gefter et al. Somatic Cell Genet, cited supra; Lerner, Yale J. Biol. Med., cited supra; Kenneth, Monoclonal Antibodies, cited supra). Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody that specifically binds the maf protein are identified by screening the hybridoma culture supernatants for such antibodies, e.g., using a standard ELISA assay.

Alternative to preparing monoclonal antibody-secreting hybridomas, a monoclonal antibody that binds to a NIP45 or a NIP45-interacting molecule can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and compounds particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Once a monoclonal antibody of interest specific for NIP45 or a NIP45-interacting molecule has been identified (e.g., either a hybridoma-derived monoclonal antibody or a recombinant antibody from a combinatorial library, including monoclonal antibodies to NIP45 or a NIP45-interacting molecule that are already known in the art), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. As discussed above, the sequences encoding the hydrophobic leaders of the light and heavy chains are removed and sequences encoding a nuclear localization signal (e.g., from SV40 Large T antigen) are linked in-frame to sequences encoding either the amino- or carboxy terminus of both the light and heavy chains. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit transcription factor activity in a cell, the expression vector encoding the NIP45-specific intracellular antibody is introduced into the cell by standard transfection methods as described hereinbefore.

c. NIP45- and NIP45-Interacting Molecule Derived Peptidic Compounds

In another embodiment, an inhibitory compound of the invention is a peptidic compound derived from the NIP45 or a NIP45-interacting molecule amino acid sequence. In particular, the inhibitory compound comprises a portion of NIP45 or a NIP45-interacting molecule (or a mimetic thereof) that mediates interaction of NIP45 or a NIP45-interacting molecule with a target molecule such that contact of NIP45 or a NIP45-interacting molecule with this peptidic compound competitively inhibits the interaction of NIP45 or a NIP45-interacting molecule with the target molecule. In an exemplary embodiment, the peptide compound is designed based on the region of NIP45 that mediates interaction of NIP45 with, for example, PRMT1. As described herein, amino acid residues 1-32 of the NIP45 protein mediate the interaction of the NIP45 proteins with PRMT1 and peptides spanning the region inhibit the ability of PRMT1 to bind to and methylate NIP45 proteins, without affecting the methylase activity of PRMT1 against other substrates.

In a preferred embodiment, a NIP45 or a NIP45-interacting molecule inhibitory compound is a peptidic compound, which is prepared based on a PRMT1-interacting region of NIP45. A peptide can be derived from the PRMT1-interacting region of NIP45 having an amino acid sequence that comprises the amino acid residues 1-32 of NIP45. Alternatively, longer or shorter regions of human NIP45 may be used such as a peptide.

The peptidic compounds of the invention can be made intracellularly in immune cells by introducing into the immune cells an expression vector encoding the peptide. Such expression vectors can be made by standard techniques, using, for example, oligonucleotides that encode the amino acid sequences of SEQ ID NO: 2. The peptide can be expressed in intracellularly as a fusion with another protein or peptide (e.g., a GST fusion). Alternative to recombinant synthesis of the peptides in the cells, the peptides can be made by chemicals synthesis using standard peptide synthesis techniques. Synthesized peptides can then be introduced into cells by a variety of means known in the art for introducing peptides into cells (e.g., liposome and the like).

Other inhibitory agents that may be used to specifically inhibit the activity of an NIP45 or a NIP45-interacting molecule protein are chemical compounds that directly inhibit NIP45 or a NIP45-interacting molecule activity or inhibit the interaction between NIP45 or a NIP45-interacting molecule and target molecules. Such compounds can be identified using screening assays that select for such compounds, as described in detail above.

B. Upregulation of Immune Responses

Stimulation of cytokine production as a means of upregulating immune responses is also useful in therapy. Upregulation of immune responses can be in the form of enhancing an existing immune response or eliciting an initial immune response. For example, enhancing an immune response through enhancing of cytokine production (in particular TH1 cyotkine production) is useful in cases of infections with microbes, e.g., bacteria (e.g., intracellular bacteria), viruses, or parasites. For example, in one embodiment, an agent that enhances NIP45 or a NIP45-interacting molecule activity, e.g., a small molecule or a NIP45 or a NIP45-interacting molecule peptide, is therapeutically useful in situations where upregulation of humoral and/or cell-mediated responses, resulting in more rapid or thorough clearance of a virus, would be beneficial. These conditions include viral skin diseases such as Herpes or shingles, in which case such an agent can be delivered topically to the skin. In addition, systemic viral diseases such as influenza, the common cold, and encephalitis might be alleviated by the administration of such agents systemically. In certain instances, it may be desirable to further administer other agents that upregulate immune responses, for example, agents that transduce signals via costimulatory receptors, in order further augment the immune response.

Alternatively, immune responses can be enhanced in an infected patient by removing immune cells from the patient, contacting immune cells in vitro with an agent (e.g., a small molecule) that enhances NIP45 or a NIP45-interacting molecule activity, and, reintroducing the in vitro-stimulated immune cells into the patient. In another embodiment, a method of enhancing immune responses involves isolating infected cells from a patient, e.g., virally infected cells, transfecting them with a nucleic acid molecule encoding a form of NIP45 or a NIP45-interacting molecule that is more active than the wild-type NIP45 or a NIP45-interacting molecule, such that the cells express all or a portion of the NIP45 or a NIP45-interacting molecule on their surface, and reintroducing the transfected cells into the patient. The transfected cells may be capable of preventing an inhibitory signal to, and thereby activating, immune cells in vivo.

An agent that enhances NIP45 or a NIP45-interacting molecule activity may be used prophylactically in therapy against various polypeptides, e.g., polypeptides derived from pathogens for vaccination. Immunity against a pathogen, e.g., a virus, can be induced by vaccinating with a viral polypeptide along with an agent that enhances NIP45 or a NIP45-interacting molecule activity. Nucleic acid vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. (1996) J. Biotechnol. 44:37)). Alternatively, nucleic acid vaccines can be administered by non-invasive means. For example, pure or lipid-formulated DNA can be delivered to the respiratory system or targeted elsewhere, e.g., Peyers patches by oral delivery of DNA (Schubbert (1997) Proc. Natl. Acad. Sci. USA 94:961). Attenuated microorganisms may be used for delivery to mucosal surfaces (Sizemore et al. (1995) Science 270:29).

Stimulation of an immune response to tumor cells can also be achieved by enhancing NIP45 or a NIP45-interacting molecule activity by treating a patient with an agent that for example, enhances NIP45-PRMT1 interaction. Preferred examples of such agents include, e.g., and compounds identified in the subject screening assays and peptides.

In another embodiment, the immune response can be stimulated by stimulating the expression and/or activity of NIP45 or a NIP45-interacting molecule. For example, immune responses against antigens to which a subject cannot mount a significant immune response, e.g., tumor-specific antigens, can be enhanced by administering an agent that stimulates the expression and/or activity of NIP45 or a NIP45-interacting molecule. Other NIP45 or a NIP45-interacting molecule agonists may be used as adjuvants to boost responses to foreign antigens in the process of active immunization.

In one embodiment, immune cells are obtained from a subject and cultured ex vivo in the presence of an agent that that enhances NIP45 or a NIP45-interacting molecule activity to expand the population of immune cells. In a further embodiment the immune cells are then administered to a subject. Immune cells can be stimulated to proliferate in vitro by, for example, providing the immune cells with a primary activation signal and a costimulatory signal, as is known in the art. Various forms of NIP45 or a NIP45-interacting molecule polypeptides or agents that enhance NIP45 or a NIP45-interacting molecule activity can also be used to costimulate proliferation of immune cells. In one embodiment immune cells are cultured ex vivo according to the method described in PCT Application No. WO 94/29436. The agent can be soluble, attached to a cell membrane or attached to a solid surface, such as a bead.

In an additional embodiment, in performing any of the methods described herein, it is within the scope of the invention to upregulate an immune response by administering one or more additional agents. For example, the use of other agents known to stimulate the immune response, such as cytokines, adjuvants, or stimulatory forms of costimulatory molecules or their ligands may be used in conjunction with an agent that enhances, e.g., NIP45 activity. Examples of other immunomodulating reagents include antibodies that provide a costimulatory signal, (e.g., agonists of CD28or ICOS), stimulating antibodies against immune cell markers, and/or cytokines and the like.

i. Exemplary Stimulatory Compounds

Since upregulation of cytokine production is associated with an increased immune response, a compound that specifically stimulates NIP45 or a NIP45-interacting molecule activity and/or expression may be used to enhance or upmodulate an immune response. In the stimulatory methods of the invention, a subject is treated with a stimulatory compound that stimulates expression and/or activity of a NIP45 or a NIP45-interacting molecule. The methods of the invention using NIP45 or a NIP45-interacting molecule stimulatory compounds may be used in the treatment of disorders in which the immune response is enhanced, promoted, stimulated, upregulated or the like.

Examples of stimulatory compounds include active NIP45 or a NIP45-interacting molecule protein, expression vectors encoding NIP45 or a NIP45-interacting molecule and chemical agents that specifically stimulate NIP45 or a NIP45-interacting molecule activity.

A preferred stimulatory compound is a nucleic acid molecule encoding NIP45 or a NIP 45-interacting molecule, wherein the nucleic acid molecule is introduced into the subject (e.g., T cells of the subject) in a form suitable for expression of the NIP45 protein in the cells of the subject. The amino acid sequence of NIP45 protein has been determined (shown in SEQ ID NO: 2) and a cDNA encoding NIP45 protein has been isolated (the nucleotide sequence of which is shown in SEQ ID NO: 1) (GI No.:1747518). The nucleotide sequence of NFATc1 is shown in SEQ ID NO:3, the amino acid sequence in SEQ ID NO:4 (GI No.:3643194). The nucleotide sequence of NFATc2 is shown in SEQ ID NO:5, the amino acid sequence in SEQ ID NO:6 (GI No.:1353236). The nucleotide sequence of NFATc3 is shown in SEQ ID NO:7, the amino acid sequence in SEQ ID NO:8 (GI No.:1906311). The nucleotide sequence of PRMT1 is shown in SEQ ID NO:9, the amino acid sequence in SEQ ID NO: 10 (GI No.:7141325).

For example, a NIP45 or a NIP45-interacting molecule cDNA (full length or partial NIP45 or a NIP45-interacting molecule cDNA sequence) is cloned into a recombinant expression vector and the vector is transfected into the immune cell using standard molecular biology techniques. The NIP45 or a NIP45-interacting molecule cDNA can be obtained, for example, by amplification using the polymerase chain reaction (PCR) or by screening an appropriate cDNA library. The nucleotide sequences of NIP45 or a NIP45-interacting molecule cDNA is known in the art and may be used for the design of PCR primers that allow for amplification of a cDNA by standard PCR methods or for the design of a hybridization probe that may be used to screen a cDNA library using standard hybridization methods.

Following isolation or amplification of NIP45 or a NIP45-interacting molecule cDNA, the DNA fragment is introduced into a suitable expression vector, as described above. Nucleic acid molecules encoding NIP45 or a NIP45-interacting molecule in the form suitable for expression of the NIP45 or a NIP45-interacting molecule in a host cell, can be prepared as described above using nucleotide sequences known in the art. The nucleotide sequences may be used for the design of PCR primers that allow for amplification of a cDNA by standard PCR methods or for the design of a hybridization probe that may be used to screen a cDNA library using standard hybridization methods.

Another form of a stimulatory compound for stimulating expression of NIP45 or a NIP45-interacting molecule in a cell is a chemical compound that specifically stimulates the expression or activity of endogenous NIP45 or a NIP45-interacting molecule in the cell. Such compounds can be identified using screening assays that select for compounds that stimulate the expression or activity of NIP45 as described herein.

The method of the invention for modulating NIP45 or a NIP45-interacting molecule activity in a subject can be practiced either in vitro or in vivo (the latter is discussed further in the following subsection). For practicing the method in vitro, cells (e.g., T cells) can be obtained from a subject by standard methods and incubated (i.e., cultured) in vitro with a stimulatory or inhibitory compound of the invention to stimulate or inhibit, respectively, the activity of NIP45 or a NIP45-interacting molecule. Methods for isolating immune cells are known in the art.

Cells treated in vitro with either a stimulatory or inhibitory compound can be administered to a subject to influence the growth and/or differentiation of immune cells in the subject. For example, immune cells can be isolated from a subject, expanded in number in vitro by enhancing NIP45 or a NIP45-interacting molecule activity in the cells using an enhancing agent (thereby promoting the proliferation of the cells), and then the immune cells can be readministered to the same subject, or another subject tissue compatible with the donor of the immune cells. Accordingly, in another embodiment, the modulatory method of the invention comprises culturing immune cells in vitro with a NIP45 or a NIP45-interacting molecule modulator and further comprises administering the immune cells to a subject to thereby modulate T growth and/or differentiation in a subject. Upon culture in vitro, the immune cells can differentiate into mature immune cells and thus the methods encompass administering this mature immune cells to the subject. For administration of cells or T to a subject, it may be preferable to first remove residual compounds in the culture from the cells or T before administering them to the subject. This can be done for example by gradient centrifugation of the cells or by washing of the T tissue. For further discussion of ex vivo genetic modification of cells followed by readministration to a subject, see also U.S. Pat. No. 5,399,346 by W. F. Anderson et al.

In other embodiments, a stimulatory or inhibitory compound is administered to a subject in vivo, such as directly to an articulation site of a subject. For stimulatory or inhibitory agents that comprise nucleic acids (e.g., recombinant expression vectors encoding NIP45 or a NIP45-interacting molecule, antisense RNA, intracellular antibodies or NIP45- or a NIP45-interacting molecule-derived peptides), the compounds can be introduced into cells of a subject using methods known in the art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo may be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm may be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271) A recombinant retrovirus can be constructed having a nucleotide sequences of interest incorporated into the retroviral genome. Additionally portions of the retroviral genome can be removed to render the retrovirus replication defective. The replication defective retrovirus is then packaged into virions which may be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 82:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and may be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 may be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. 1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

### VI. DIAGNOSTIC ASSAYS

In another aspect, the invention features a method of diagnosing a subject for a disorder that would benefit from modulation of the activity of one or more of: a NIP45-binding molecule, e.g., a PRMT (such as a NIP45 related activity of a PRMT); or from modulation of NIP45. Exemplary disorders include those that would benefit from modulation of cytokine production, e.g., modulation of IL-2, IFN-γ, IL-4, IL-5 and/or IL-13 production, modulation of the relative number of Th1 and Th2 cells, modulation of effector T cell function, and modulation of T cell differentiation.

In one embodiment, the expression of NIP45 or a NIP45-interacting molecule or a molecule in cells of a subject may be measured and compared to a control and a difference in expression of NIP45 or a NIP45-interacting molecule in cells of the subject as compared to the control could be used to diagnose the subject as one that would benefit from modulation of cytokine activity.

The “change in expression” or “difference in expression” may be detected by assaying levels of mRNA, for example, by isolating cells from the subject and determining the level of mRNA expression in the cells by standard methods known in the art, including Northern blot analysis, microarray analysis, reverse-transcriptase PCR analysis and in situ hybridizations. For example, a biological specimen can be obtained from the patient and assayed for, e.g., expression or activity of NIP45 or a NIP45-interacting molecule.

In another embodiment, protein expression may be measured using standard methods known in the art, including Western blot analysis, immunoprecipitations, enzyme linked immunosorbent assays (ELISAs) and immunofluorescence. Antibodies for use in such assays can be made using techniques known in the art and/or as described herein for making intracellular antibodies.

In another embodiment, a change in expression of NIP45 or a NIP45-interacting molecule or a molecule in a signal transduction pathway involving NIP45 or a NIP45-interacting molecule in cells of the subject results from one or more mutations (i.e., alterations from wild-type), e.g., the NIP45 or a NIP45-interacting molecule gene and mRNA leading to one or more mutations (i.e., alterations from wild-type) in the amino acid sequence of the protein. In one embodiment, the mutation(s) leads to a form of the molecule with increased activity (e.g., partial or complete constitutive activity). In another embodiment, the mutation(s) leads to a form of the molecule with decreased activity (e.g., partial or complete inactivity). The mutation(s) may change the level of expression of the molecule for example, increasing or decreasing the level of expression of the molecule in a subject with a disorder. Alternatively, the mutation(s) may change the regulation of the protein, for example, by modulating the interaction of the mutant protein with one or more targets e.g., resulting in a form of NIP45 that cannot be methylated or cannot interact with a NIP45-binding partner or resulting in a form that is unable to methylate. Mutations in the nucleotide sequence or amino acid sequences of proteins can be determined using standard techniques for analysis of DNA or protein sequences, for example for DNA or protein sequencing, RFLP analysis, and analysis of single nucleotide or amino acid polymorphisms. For example, in one embodiment, mutations can be detected using highly sensitive PCR approaches using specific primers flanking the nucleic acid sequence of interest. In one embodiment, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). This method can include the steps of collecting a sample of cells from a patient, isolating nucleic acid (e.g., genomic, DNA) from the cells of the sample, contacting the nucleic acid sample with one or more primers which specifically amplify a sequence under conditions such that hybridization and amplification of the sequence (if present) occurs, and detecting the presence or absence of an amplification product, or detecting the size of the amplification product and comparing the length to a control sample.

In one embodiment, the complete nucleotide sequence for NIP45 or a NIP45-interacting molecule or a molecule in a signal transduction pathway involving NIP45 or a NIP 45-interacting molecule can be determined. Particular techniques have been developed for determining actual sequences in order to study polymorphism in human genes. See, for example, Proc. Natl. Acad. Sci. U.S.A. 85, 544-548 (1988) and Nature 330, 384-386 (1987); Maxim and Gilbert. 1977. PNAS 74:560; Sanger 1977. PNAS 74:5463. In addition, any of a variety of automated sequencing procedures can be utilized when performing diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Restriction fragment length polymorphism mappings (RFLPS) are based on changes at a restriction enzyme site. In one embodiment, polymorphisms from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Moreover, the use of sequence specific ribozymes (see, for example, U.S. Pat. No. 5,498,531) may be used to score for the presence of a specific ribozyme cleavage site.

Another technique for detecting specific polymorphisms in particular DNA segment involves hybridizing DNA segments which are being analyzed (target DNA) with a complimentary, labeled oligonucleotide probe. See Nucl. Acids Res. 9, 879-894 (1981). Since DNA duplexes containing even a single base pair mismatch exhibit high thermal instability, the differential melting temperature may be used to distinguish target DNAs that are perfectly complimentary to the probe from target DNAs that only differ by a single nucleotide. This method has been adapted to detect the presence or absence of a specific restriction site, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for detecting polymorphisms in nucleic acid sequences include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the polymorphic sequence with potentially polymorphic RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In another embodiment, alterations in electrophoretic mobility may be used to identify polymorphisms. For example, single strand conformation polymorphism (SSCP) may be used to detect differences in electrophoretic mobility between mutant and wild-type nucleic acids (Orita et al. (1989) Proc Natl. Acad. Sci. USA: 86:2766, see also Cotton (1993) Mutat Res 285:125-144; and Hayashi (1992) Genet Anal Tech Appl 9:73-79). Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of a nucleic acid molecule comprising polymorphic sequences in polyacrylamide gels containing a gradient of denaturant is assayed using denaturing gradient gel electrophoresis (DGGE) (Myers et al. 1985) Nature 313:495). When DGGE is used as the method of analysis, DNA can be modified to insure that it does not completely denature, for example by adding a GC clamp of approximately 40 bp, of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting polymorphisms include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different polymorphisms when the oligonucleotides are attached to the hybridizing membrane and hybridized with labeled target DNA.

Another process for studying differences in DNA structure is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Another process exploits the fact that the incorporation of some nucleotide analogs into DNA causes an incremental shift of mobility when the DNA is subjected to a size fractionation process, such as electrophoresis. Nucleotide analogs may be used to identify changes since they can cause an electrophoretic mobility shift. See, U.S. Pat. No. 4,879,214.

Many other techniques for identifying and detecting polymorphisms are known to those skilled in the art, including those described in “DNA Markers: Protocols, Applications and Overview,” G. Caetano-Anolles and P. Gresshoff ed., (Wiley-VCH, New York) 1997, which is incorporated herein by reference as if fully set forth.

In addition, many approaches have also been used to specifically detect SNPs. Such techniques are known in the art and many are described e.g., in DNA Markers: Protocols, Applications, and Overviews. 1997. Caetano-Anolles and Gresshoff, Eds. Wiley-VCH, New York, pp 199-211 and the references contained therein). For example, in one embodiment, a solid phase approach to detecting polymorphisms such as SNPs may be used. For example an oligonucleotide ligation assay (OLA) may be used. This assay is based on the ability of DNA ligase to distinguish single nucleotide differences at positions complementary to the termini of co-terminal probing oligonucleotides (see, e.g., Nickerson et al. 1990. Proc. Natl. Acad. Sci. USA 87:8923. A modification of this approach, termed coupled amplification and oligonucleotide ligation (CAL) analysis, has been used for multiplexed genetic typing (see, e.g., Eggerding 1995 PCR Methods Appl. 4:337); Eggerding et al. 1995 Hum. Mutat. 5:153).

In another embodiment, genetic bit analysis (GBA) may be used to detect a SNP (see, e.g., Nikiforov et al. 1994. Nucleic Acids Res. 22:4167; Nikiforov et al. 1994. PCR Methods Appl. 3:285; Nikiforov et al. 1995. Anal Biochem. 227:201). In another embodiment, microchip electrophoresis may be used for high-speed SNP detection (see e.g., Schmalzing et al. 2000. Nucleic Acids Research, 28). In another embodiment, matrix-assisted laser desorption/ionization time-of-flight mass (MALDI TOF) mass spectrometry may be used to detect SNPs (see, e.g., Stoerker et al. Nature Biotechnology 18:1213).

In another embodiment, a difference in a biological activity of NIP45 or a NIP45-interacting molecule between a subject and a control can be detected. For example, an activity of NIP45 or a NIP45-interacting molecule or a molecule in a signal transduction pathway involving NIP45 or a NIP45-interacting molecule can be detected in cells of a subject suspected of having a disorder associated with aberrant biological activity of NIP45. The activity of NIP45 or a NIP45-interacting molecule or a molecule in a signal transduction pathway involving NIP45 or a NIP45-interacting molecule in cells of the subject could then be compared to a control and a difference in activity of NIP45 or a NIP45-interacting molecule or a molecule in a signal transduction pathway involving NIP45 or a NIP45-interacting molecule in cells of the subject as compared to the control could be used to diagnose the subject as one that would benefit from modulation of an NIP45 or a NIP 45-interacting molecule activity. Activities of NIP45 or a NIP45-interacting molecule or molecules in a signal transduction pathway involving NIP45 or a NIP45-interacting molecule can be detected using methods described herein or known in the art.

In preferred embodiments, the diagnostic assay is conducted on a biological sample from the subject, such as a cell sample or a tissue section (for example, a freeze-dried or fresh frozen section of tissue removed from a subject). In another embodiment, the level of expression of NIP45 or a NIP45-interacting molecule or a molecule in a signal transduction pathway involving NIP45 or a NIP45-interacting molecule in cells of the subject can be detected in vivo, using an appropriate imaging method, such as using a radiolabeled antibody.

In one embodiment, the level of expression of NIP45 or a NIP45-interacting molecule or a molecule in a signal transduction pathway involving NIP45 or a NIP45-interacting molecule in cells of the test subject may be elevated (i.e., increased) relative to the control not associated with the disorder or the subject may express a constitutively active (partially or completely) form of the molecule. This elevated expression level of, e.g., NIP45 or a NIP45-interacting molecule or expression of a constitutively active form of NIP45 or a NIP45-interacting molecule, may be used to diagnose a subject for a disorder associated with increased NIP45 or a NIP45-interacting molecule activity.

In another embodiment, the level of expression of NIP45 or a NIP45-interacting molecule or a molecule in a signal transduction pathway involving NIP45 or a NIP45-interacting molecule in cells of the subject may be reduced (i.e., decreased) relative to the control not associated with the disorder or the subject may express an inactive (partially or completely) mutant form of NIP45 or a NIP45-interacting molecule. This reduced expression level of NIP45 or a NIP45-interacting molecule or expression of an inactive mutant form of NIP45 or a NIP45-interacting molecule may be used to diagnose a subject for a disorder, such as immunodeficiency disorders characterized by insufficient cytokine production.

In one embodiment, the level of expression of gene whose expression is regulated by NIP45 or a NIP45-interacting molecule can be measured (e.g., IL-4).

In another embodiment, an assay diagnosing a subject as one that would benefit from modulation of cytokine expression, post-translational modification, and/or activity (or a molecule in a signal transduction pathway involving NIP45 or a NIP45-interacting molecule) is performed prior to treatment of the subject.

The methods described herein may be performed, for example, by utilizing pre-packaged diagnostic kits comprising at least one probe/primer nucleic acid or other reagent (e.g., antibody), which may be conveniently used, e.g., in clinical settings to diagnose patients exhibiting symptoms or family history of a disease or illness involving cytokine production, NIP45 or a NIP45-interacting molecule or a molecule in a signal transduction pathway involving NIP45 or a NIP45-interacting molecule.

### VII. KITS OF THE INVENTION

Another aspect of the invention pertains to kits for carrying out the screening assays, modulatory methods or diagnostic assays of the invention. For example, a kit for carrying out a screening assay of the invention may include an indicator composition comprising NIP45 or a NIP45-interacting molecule, means for measuring a readout (e.g., protein secretion) and instructions for using the kit to identify modulators of biological effects of NIP45 or a NIP45-interacting molecule. In another embodiment, a kit for carrying out a screening assay of the invention may include cells deficient in NIP45 or a NIP45-interacting molecule, means for measuring the readout and instructions for using the kit to identify modulators of a biological effect of NIP45 or a NIP45-interacting molecule, e.g., cytokine production.

In another embodiment, the invention provides a kit for carrying out a modulatory method of the invention. The kit can include, for example, a modulatory agent of the invention (e.g., NIP45 or a NIP45-interacting molecule inhibitory or stimulatory agent) in a suitable carrier and packaged in a suitable container with instructions for use of the modulator to modulate a biological effect of NIP45 or a NIP45-interacting molecule, e.g., cytokine production.

Another aspect of the invention pertains to a kit for diagnosing a disorder associated with a cytokine production, expression and/or activity in a subject. The kit can include a reagent for determining expression of NIP45 or a NIP45-interacting molecule (e.g., a nucleic acid probe for detecting NIP45 or a NIP45-interacting molecule mRNA or an antibody for detection of NIP45 or a NIP45-interacting molecule protein), a control to which the results of the subject are compared, and instructions for using the kit for diagnostic purposes.

### VIII. IMMUNOMODULATORY COMPOSITIONS

Agents that modulate a NIP45 or a NIP45-interacting molecule activity, (e.g., that modulate one or more of the expression, processing, post-translational modifications, or activity, expression, processing, post-translational modification NIP45or a NIP45-interacting molecule) are also appropriate for use in immunomodulatory compositions. Stimulatory or inhibitory agents of the invention may be used to up or down regulate the immune response in a subject.

The modulating agents of the invention can be given alone, or in combination with an antigen to which an enhanced immune response or a reduced immune response is desired.

In one embodiment, agents which are known adjuvants can be administered with the subject modulating agents. At this time, the only adjuvant widely used in humans has been alum (aluminum phosphate or aluminum hydroxide). Saponin and its purified component Quil A, Freund's complete adjuvant, and other adjuvants used in research and veterinary applications have potential use in human vaccines. However, new chemically defined preparations such as muramyl dipeptide, monophosphoryl lipid A, phospholipid conjugates such as those described by Goodman-Snitkoff et al. J. Immunol. 147:410-415 (1991) resorcinols, non-ionic surfactants such as polyoxyethylene oleyl ether and n-hexadecyl polyethylene ether, enzyme inhibitors include pancreatic trypsin inhibitor, diisopropylfluorophosphate (DEP) and trasylol can also be used. In embodiments in which antigen is administered, the antigen can e.g., be encapsulated within a proteoliposome as described by Miller et. al., J. Exp. Med. 176:1739-1744 (1992) and incorporated by reference herein, or in lipid vesicles; such as Novasome™ lipid vesicles (Micro Vescular Systems, Inc., Nashua, N.H.), to further enhance immune responses.

In one embodiment, a nucleic acid molecule encoding NIP45 or a NIP45-interacting molecule (e.g., a sense or antisense or siRNA molecule or a NIP45-interacting molecule or portion thereof) is administered as a DNA vaccine. This can be done using a plasmid DNA construct which is similar to those used for delivery of reporter or therapeutic genes. Such a construct preferably comprises a bacterial origin of replication that allows amplification of large quantities of the plasmid DNA; a prokaryotic selectable marker gene; a nucleic acid sequence encoding, e.g., a NIP45 polypeptide or portion thereof; eukaryotic transcription regulatory elements to direct gene expression in the host cell; and a polyadenylation sequence to ensure appropriate termination of the expressed mRNA (Davis. 1997. Curr. Opin. Biotechnol. 8:635). Vectors used for DNA immunization may optionally comprise a signal sequence (Michel et al. 1995. Proc. Natl. Acad. Sci. USA. 92:5307; Donnelly et al. 1996. J. Infect Dis. 173:314). DNA vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. 1996. J. Biotechnol. 44:37)). Alternatively, DNA vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms may be used for delivery to mucosal surfaces. (Sizemore et al. 1995. Science. 270:29)

In one embodiment, plasmids for DNA vaccination can express NIP45 or a NIP45-interacting molecule (or antagonist of NIP45 or a NIP45-interacting molecule as well as the antigen against which the immune response is desired or can encode modulators of immune responses such as lymphokine genes or costimulatory molecules (Iwasaki et al. 1997. J. Immunol. 158:4591).

### IX. ADMINISTRATION OF MODULATING AGENTS

Modulatory agents of the invention are administered to subjects in a biologically compatible form suitable for pharmaceutical administration in vivo to either enhance or suppress immune responses (e.g., T cell mediated immune responses). By “biologically compatible form suitable, for administration in vivo” is meant a form of the protein to be administered in which any toxic effects are outweighed by the therapeutic effects of the modulating agent. The term subject is intended to include living organisms in which an immune response can be elicited, e.g., mammals. Examples of subjects include humans, dogs, cats, mice, rats, and transgenic species thereof, including but not limited to the transgenic NIP45 or a NIP45-interacting molecule mouse described herein. Administration of an agent as described herein can be in any pharmacological form including a therapeutically active amount of an agent alone or in combination with a pharmaceutically acceptable carrier.

Administration of a therapeutically active amount of the therapeutic compositions of the present invention is defined as an amount effective, at dosages and for periods of time necessary to achieve the desired result. For example, a therapeutically active amount of a cytokine production modulating agent may vary according to factors such as the disease state, age, sex, and weight of the individual, and the ability of peptide to elicit a desired response in the individual. Dosage regimen may be adjusted to provide the optimum therapeutic response. For example, several divided doses may be administered daily or the dose may be proportionally reduced as indicated by the exigencies of the therapeutic situation.

The therapeutic or pharmaceutical compositions of the present invention can be administered by any suitable route known in the art including for example intravenous, subcutaneous, intramuscular, transdermal, intrathecal or intracerebral or administration to cells in ex vivo treatment protocols. Administration can be either rapid as by injection or over a period of time as by slow infusion or administration of slow release formulation. For treating tissues in the central nervous system, administration can be by injection or infusion into the cerebrospinal fluid (CSF). When it is intended that a cytokine production modulator be administered to cells in the central nervous system, administration can be with one or more agents capable of promoting penetration of cytokine production modulator across the blood-brain barrier.

The cytokine production modulator can also be linked or conjugated with agents that provide desirable pharmaceutical or pharmacodynamic properties. For example, cytokine production modulator can be coupled to any substance known in the art to promote penetration or transport across the blood-brain barrier such as an antibody to the transferrin receptor, and administered by intravenous injection. (See for example, Friden et al., 1993, Science 259:373-377 which is incorporated by reference). Furthermore, cytokine production modulator can be stably linked to a polymer such as polyethylene glycol to obtain desirable properties of solubility, stability, half-life and other pharmaceutically advantageous properties. (See for example Davis et al., 1978, Enzyme Eng 4:169-73; Burnham, 1994, Am J Hosp Pharm 51:210-218, which are incorporated by reference).

Furthermore, the cytokine production modulator can be in a composition which aids in delivery into the cytosol of a cell. For example, the agent may be conjugated with a carrier moiety such as a liposome that is capable of delivering the peptide into the cytosol of a cell. Such methods are well known in the art (for example see Amselem et al., 1993, Chem Phys Lipids 64:219-237, which is incorporated by reference). Alternatively, the NIP45 or a NIP45-interacting molecule modulator can be modified to include specific transit peptides or fused to such transit peptides which are capable of delivering the cytokine production modulator into a cell. In addition, the agent can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. NIP45 or a NIP45-interacting molecule can also be incorporated into a solid or semi-solid biologically compatible matrix which can be implanted into tissues requiring treatment.

The carrier can also contain other pharmaceutically-acceptable excipients for modifying or maintaining the pH, osmolality, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used. It is also provided that certain formulations containing the cytokine production modulator are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, olyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies may be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method for the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In one embodiment of this invention, a modulatory agent may be therapeutically administered by implanting into patients vectors or cells capable of producing, for example a biologically-active form of NIP45 or a NIP45-interacting molecule or a precursor of NIP45 or a NIP45-interacting molecule, i.e. a molecule that can be readily converted to a biological-active form of or a NIP45-interacting molecule by the body. In one approach cells that secrete NIP45 or a NIP45-interacting molecule may be encapsulated into semipermeable membranes for implantation into a patient. The cells can be cells that normally express NIP45 or a NIP45-interacting molecule or a precursor thereof or the cells can be transformed to express NIP45 or a NIP45-interacting molecule or a biologically active fragment thereof or a precursor thereof. It is preferred that the cell be of human origin and that the NIP45 or a NIP45-interacting molecule polypeptide be human NIP45 or a NIP45-interacting molecule when the patient is human. However, the formulations and methods herein may be used for veterinary as well as human applications and the term “patient” or “subject” as used herein is intended to include human and veterinary patients.

Monitoring the influence of agents (e.g., drugs or compounds) on the expression or activity of a cytokine gene can be applied not only in basic drug screening, but also in clinical trials. For example, the effectiveness of an agent determined by a screening assay as described herein to increase cytokine gene expression, protein levels, or upregulate cytokine activity, can be monitored in clinical trials of subjects exhibiting decreased cytokine gene expression, protein levels, or downregulated cytokine activity. Alternatively, the effectiveness of an agent determined by a screening assay to decrease cytokine gene expression, protein levels, or downregulate cytokine activity, can be monitored in clinical trials of subjects exhibiting increased cytokine gene expression, protein levels, or upregulated cytokine activity. In such clinical trials, the expression or activity of a cytokine gene, and preferably, other genes that have been implicated in a disorder may be used as a “read out” or markers of the phenotype of a particular cell.

For example, and not by way of limitation, genes, including NIP45 and NIP45-interacting molecules, that are modulated in cells by treatment with an agent (e.g., compound, drug or small molecule) which modulates NIP45 or NIP45-interacting molecule activity (e.g., identified in a screening assay as described herein) can be identified. Thus, to study the effect of agents on a NIP45 or NIP45-interacting molecule associated disorder, for example, in a clinical trial, cells can be isolated and RNA prepared and analyzed for the levels of expression of NIP45 or NIP45-interacting molecule and other genes implicated in the NIP45 associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NIP45 or NIP45-interacting molecule or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a cytokine protein, mRNA, or genomic DNA in the pre-administration sample; (iii) obtaining one or more post-administration samples from the subject; (iv) detecting the level of expression or activity of the NIP45 or NIP45-interacting molecule protein, mRNA, or genomic DNA in the post-administration samples; (v) comparing the level of expression or activity of the NIP45 or NIP45-interacting molecule protein, mRNA, or genomic DNA in the pre-administration sample with the NIP45 or NIP45-interacting molecule protein, mRNA, or genomic DNA in the post administration sample or samples; and (vi) altering the administration of the agent to the subject accordingly. For example, increased administration of the agent may be desirable to increase the expression or activity of NIP45 or NIP45-interacting molecule to higher levels than detected, i.e., to increase the effectiveness of the agent. Alternatively, decreased administration of the agent may be desirable to decrease expression or activity of NIP45 or NIP45-interacting molecule to lower levels than detected, i.e. to decrease the effectiveness of the agent. According to such an embodiment, NIP45 or NIP45-interacting molecule expression or activity may be used as an indicator of the effectiveness of an agent, even in the absence of an observable phenotypic response.

In a preferred embodiment, the ability of a cytokine modulating agent to modulate inflammation or apoptosis in a epithelial cell of a subject that would benefit from modulation of the expression and/or activity of a cytokine gene can be measured by detecting an improvement in the condition of the patient after the administration of the agent. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

It is likely that the level of a cytokine may be altered in a variety of conditions and that quantification of cytokine levels would provide clinically useful information. Furthermore, because it has been demonstrated herein that increased levels of cytokine expressed by a cell can shift the cell death regulatory mechanism of that cell to decrease viability, it is believed that measurement of the level of cytokine in a cell or cells such as in a group of cells, tissue or neoplasia, or the like will provide useful information regarding apoptotic state of that cell or cells.

Furthermore, in the treatment of disease conditions, compositions containing

cytokine production modulators can be administered exogenously and it would likely be desirable to achieve certain target levels of cytokine production modulators polypeptide in sera, in any desired tissue compartment or in the affected tissue. It would, therefore, be advantageous to be able to monitor the levels, of NIP45 polypeptide in a patient or in a biological sample including a tissue biopsy sample obtained form a patient and, in some cases, also monitoring the levels of NIP45 and, in some circumstances, also monitoring levels of TRAF, NFAT and/or PRMT or another NIP45-interacting polypeptide. Accordingly, the present invention also provides methods for detecting the presence of NIP45 or NIP45-interacting molecule in a sample from a patient.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods in Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

This invention is further illustrated by the following examples which should not be construed as limiting. The contents of all references, patents and published patent applications cited throughout this application are hereby incorporated by reference. Nucleotide and amino acid sequences deposited in public databases as referred to herein are also hereby incorporated by reference.

### EXAMPLE 1

### Isolation of a NIP45 cDNA Using a Yeast Two-Hybrid Interaction Trap Assay

A yeast two-hybrid interaction trap assay was used to isolate proteins that could directly bind to the RHD of NFATp. An NFATp(RHD)-Gal4 fusion protein was prepared for use as the “bait” in the yeast two-hybrid assay by cloning a 900 bp fragment of murine NFATp (McCaffrey, P. G. et al. (1993) Science 262:750-754), spanning amino acids 228 to 520, into the BamHI site of vector pEG202 (Gyuris, J. et al. (1993) Cell 75:791-803). In frame fusion of the NFAT(p) polypeptide sequences to the Gal4 sequences was confirmed by DNA sequence analysis. This bait was used to screen a cDNA library prepared from the murine T cell line D10, constructed in the plasmid pJG4-5, to select for clones encoding polypeptides that interacted with the bait, using methodologies known in the art (see Gyuris, J. et al. (1993) Cell 75:791-803).

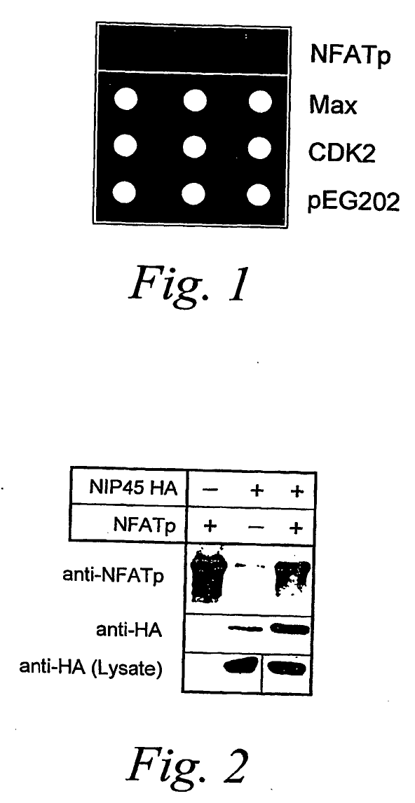

One class of interactors encoding a fusion protein with apparently high affinity for the NFATp(RHD)-Gal4 bait, as exhibited by high level of β-galactosidase activity and ability to confer leucine prototrophy, was isolated and termed NIP45 (NFAT Interacting Protein 45). FIG. 1 shows a photograph of yeast colonies (three representatives for each, plasmid combination), cotransformed with the NIP45 plasmid and either the NFATp-RHD bait or control baits (Max-Gal4, CDK2-Gal4 and the control vector pEG202, expressing only an epitope tagged Gal4 protein), together with the LacZ reporter plasmid pSH18. The yeast colonies had been selected on appropriate media, and were spotted onto plates containing Xgal and the nonrepressing carbon source galactose. Yeast colonies cotransformed with the NIP45 plasmid and the NFATp-RHD bait were blue in color, demonstrating expression of the LacZ reporter plasmid (indicative of NIP45/NFATp-RHD interaction), whereas yeast colonies transformed with the NIP45 plasmid and the control baits were white in color, indicating no interaction of NIP45 with the control baits. Transformants were also tested on galactose containing media lacking leucine, and only those containing the NIP45 plasmid and the NFATp-RHD bait grew, further indicating the specific interaction of NIP45 with NFATp-RHD. The NIP45 cDNA isolated by the two-hybrid assay was a 1.9 kb DNA fragment.

### EXAMPLE 2

### Interaction of NIP45 and NEATp In Vivo in Mammalian Cells

The ability of the NIP45 polypeptide to interact specifically with NFATp in vivo was tested in mammalian cells. The 1.9 kb NIP45 cDNA insert selected in the yeast two-hybrid system (described in Example 1) was subcloned into a mammalian expression vector which fuses the coding region to an epitope tag from a influenza hemagglutinin (HA) peptide, vector pCEP4-HA (Herrscher, R. F. et al. (1995) Genes Dev. 9:3067-3082), to create the expression vector NIP45-HA. This tagged construct was then cotransfected with an NFATp expression plasmid into HepG2 cells (which express low levels of NFATp). As controls, HepG2 cells also were cotransfected with NIP45-HA along with the parental expression vector for the NFATp construct (i.e., the expression vector without the NFATp insert) or with the NFATp expression vector along with an out of frame fusion of NIP45 with the epitope tag. Lysates were prepared from the transfected cells and immunoprecipitated with anti-NFATp antibody. Western blot analysis was then performed on the immunoprecipitated material using either anti-NFATp or anti-HA antibodies.

The results of this experiment are shown in FIG. 2. Western blot analysis of these samples using an HA-specific monoclonal antibody (mAb) demonstrated that the anti-NFATp antibody used for immunoprecipitation coimmunoprecipitated the HA-tagged NIP45 polypeptide. The lane showing transfection with only NIP45-HA (middle lane) reveals the low endogenous level of NFATp present in these cells. The amount of HA-tagged NIP45 protein immunoprecipitated was further increased by cotransfection with the NFATp expression plasmid demonstrating the specificity of this interaction (right lane). Western blot analysis of untreated lysates demonstrated that equivalent levels of NIP45-HA polypeptide were expressed in the samples tested for coimmunoprecipitation of NIP45-HA anti-NFATp antibodies. Furthermore, no immunoreactive material for either NFATp or the HA tagged protein was detected when performing immunoprecipitation using normal rabbit serum. These experiments demonstrate that NFAT and NIP45 physically associate in vivo in mammalian cells.

### EXAMPLE 3

### Structural Analysis of NIP45 cDNAs

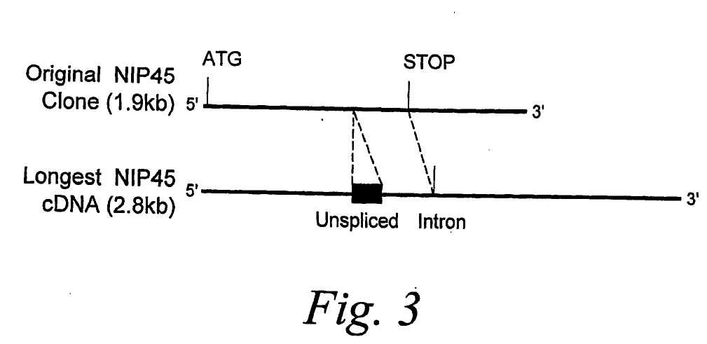

The 1.9 kb NIP45 cDNA insert from the clone isolated using the two-hybrid assay (described in Example 1) was used to screen a D10.G4 T cell lambda zap II cDNA library (Stratagene) to identify full length clones. Screening of a library containing approximately 8×10⁵ clones yielded 7 hybridizing clones most of which did not extend as far towards the 5′ end as the original isolate. Sequence analysis of the longest clone (2.8 kb), however, demonstrated identity to the original clone at the 5′ end. The structures of the original 1.9 kb cDNA isolate and the longest 2.8 kb cDNA isolate are compared in FIG. 3. The 2.8 kb cDNA isolate contained an additional segment of 180 bp located 868 bp downstream from the 5′ end of the original clone. Junction sequences at the ends of this 180 nucleotide segment indicate it to be an unspliced intron and conceptual translation of the nucleotide sequence within this region revealed an in-frame stop codon. Much of the additional sequence in this clone was at the 3′ end and represented an extensive 3′ untranslated region followed by a poly-A⁺ tail (see FIG. 3). Such extensive 3′ untranslated regions have been observed in many genes. Allowing for the splicing of the small intron and translation of the single large open reading frame, the 2.8 kb cDNA clone is predicted to encode an identical polypeptide to that of the original 1.9 kb isolate.

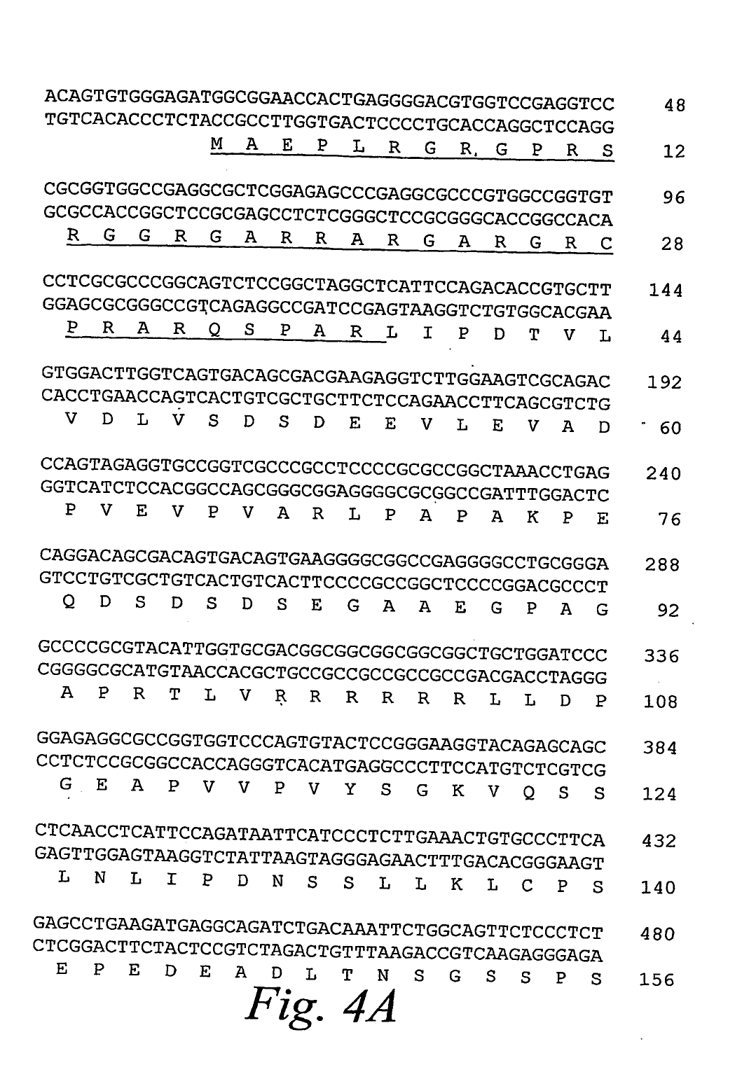

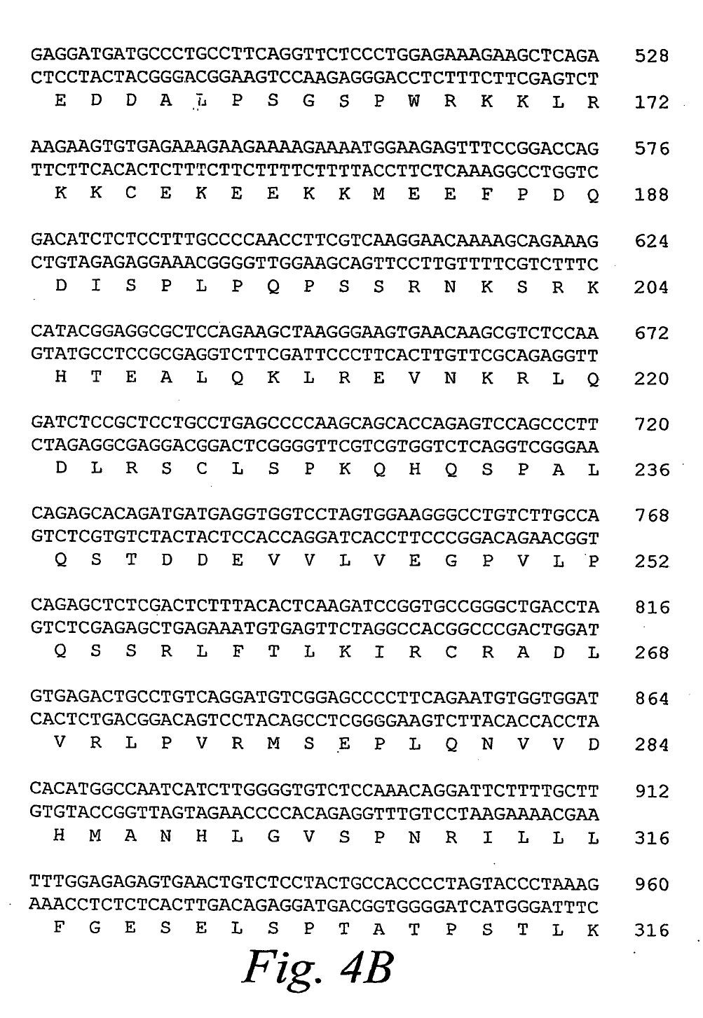

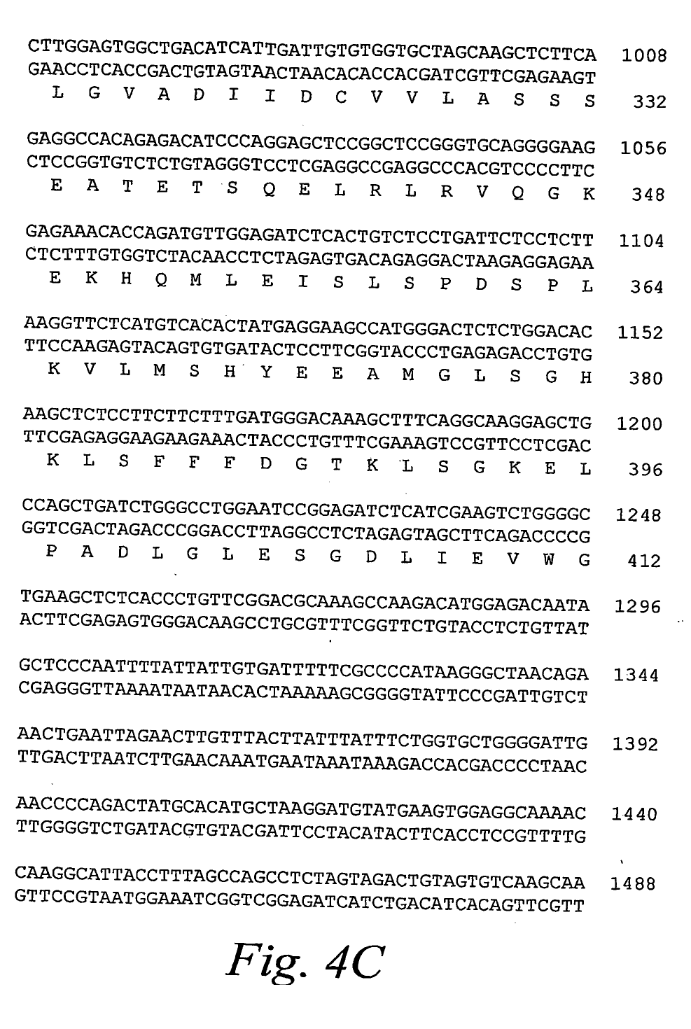

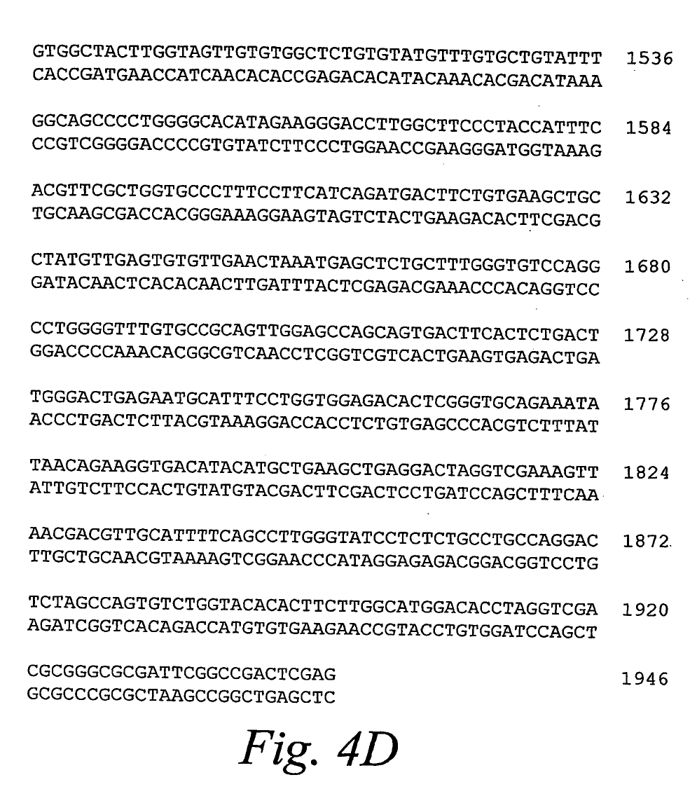

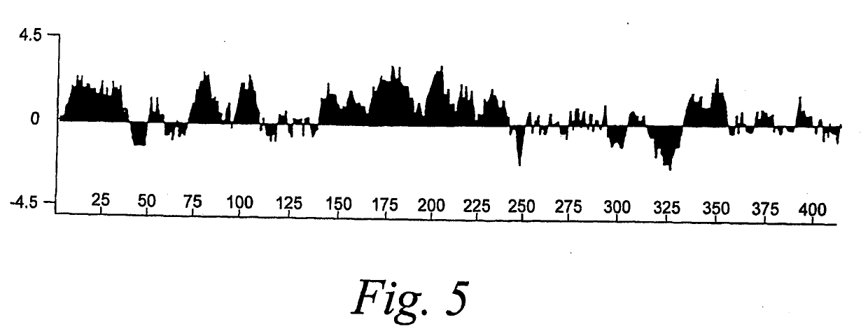

The nucleotide and predicted amino acid sequences of the 1.9 kb cDNA isolate are shown in FIG. 4 (and in SEQ ID NOs: 1 and 2, respectively). The coding region is shown from the first initiation codon through the first in frame stop codon. The nucleotide and amino acid positions are indicated to the right of the primary sequence. Conceptual translation of the 1.9 kb nucleotide sequence predicted a polypeptide of 412 amino acids with a molecular mass of 45 Kd, and hence the protein has been termed NFAT Interacting Protein 45 (NIP45). Inspection of the amino acid sequence of NIP45 revealed a highly basic domain at the N-terminus, in which 13 of 32 amino acid are basic. This region is underlined in FIG. 4. This basic region appears as a hydrophilic stretch in the hydrophobicity plot shown in FIG. 5.

### EXAMPLE 4

### Tissue Expression of NIP45 mRNA

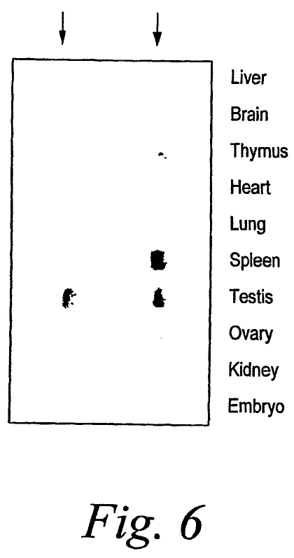

Northern blot analysis of RNA from different murine tissues was performed to investigate the tissue expression of NIP45 mRNA. 10 μg of total RNA from various tissues was separated on denaturing agarose gels, blotted and hybridized with a radiolabelled 1.4 kb NIP45 cDNA fragment. Samples were controlled for equivalent loading of RNA by comparison of ethidium bromide fluorescence. The results of the Northern blot analysis are shown in FIG. 6. The hybridizations revealed a transcript of approximately 3.1 kb, which is of comparable size to the longest cDNA clones. RNA from testis contained an additional 1.4 Kb hybridizing species. The highest levels of NIP45 transcripts were seen in spleen, thymus and testis. The preferential expression in lymphoid organs may indicate a specific function for NIP45 in the immune system. The low intensity hybridization signal and the rare occurrence of NIP45 cDNA clones in the T cell cDNA library indicate that the NIP45 RNA is a relatively rare message.

### EXAMPLE 5

### Subcellular Localization of NIP45

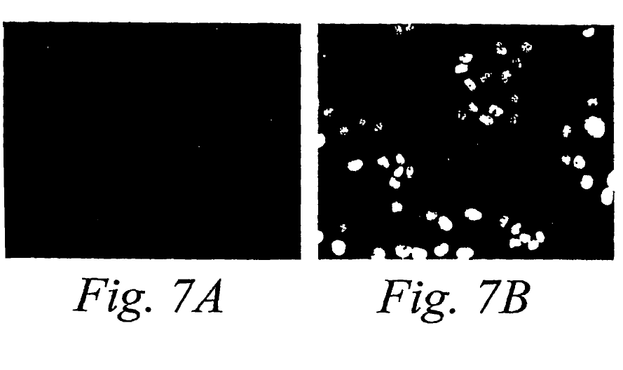

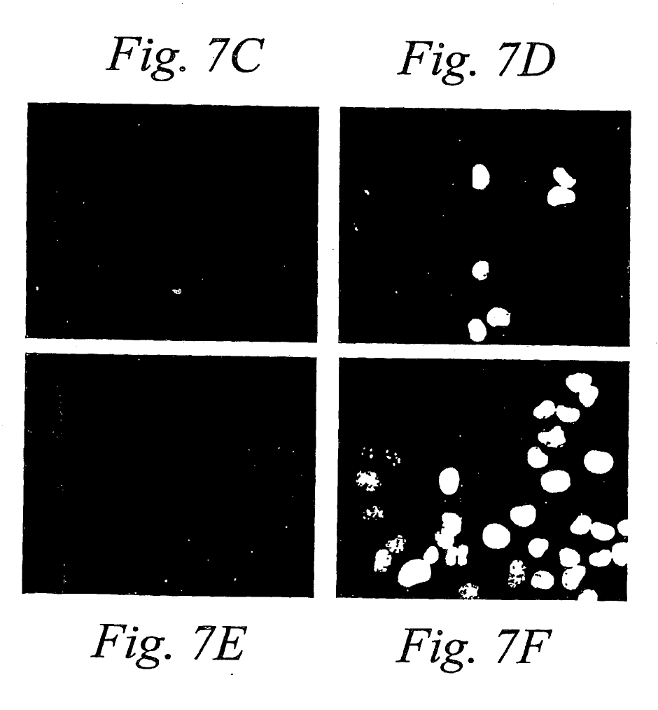

Subcellular localization of epitope tagged NIP45 protein was determined by indirect immunofluorescence. BHK cells were transfected with 1 μg of an expression construct encoding an HA-epitope tagged NIP45 (pCEP4-HA), using methodologies known in the art (see Heald, R. et al. (1993) Cell 74:463-474). Transfected cells were incubated overnight, fixed, permeabilized as described (Heald, R. et al. (1993) supra) and probed with an anti-HA mAb 12CA5 (Boehringer Mannheim) plus indocarbocyanine labeled donkey anti-mouse antibody (Jackson ImmunoResearch) and then counterstained with the dye Hoechst 33258. The results are shown in FIGS. 7A-B. Nuclear staining of NIP45 was observed with the indocarbocyanine labeled secondary reagent (see FIG. 7A) by comparison to the same cells counterstained with the DNA staining dye Hoechst 33258 (see FIG. 7B). The fluorescence pattern indicates that NIP45 is evenly distributed throughout the nucleus. Furthermore, this pattern matched that seen for cells transfected with NFAT4 and stimulated with ionomycin (Shibasaki, F. et al. (1996) Nature 382:370-373; see also below). Stimulation with PMA and/or ionomycin did not affect the subcellular localization of this NIP45.

Control experiments were also performed on BHK cells transfected with NFAT4. Cells were incubated overnight in culture media and either fixed directly or first stimulated with 1 mM ionomycin for 10 minutes before fixation and then processed as described above. The results are shown in FIGS. 7C-F. Unstimulated (FIGS. 7C and 7D) or ionomycin treated (FIGS. 7E and 7F) NFAT4 transfectants were probed with an anti-NFAT4 specific antibody followed by a indocarbocyanine labeled secondary reagent and Hoechst 33258. Indocarbocyanine fluorescence demonstrates the pattern of staining for cytoplasmic localized NFAT4 in unstimulated transfectants (FIG. 7C) and nuclear localized NFAT4 in stimulated cells (FIG. 7E). Adjacent panels (FIGS. 7D and 7F, respectively) show the same field exposed for detection of nuclei by staining with Hoechst 33258.

The effect of NIP45 on the nuclear translocation of NFAT4 also was investigated. HepG2 cells were transfected with either NFAT4 or NFAT4 plus NIP45 and stimulated the following day with 1 μM ionomycin for 0, 2, 4, 8 or 15 minutes. For one sample, the cells were stimulated for 15 minutes with ionomycin and then washed with fresh media and allowed to rest for an additional 15 minutes (indicated as “15 min.⁺15 min. rest” in Table 1). This analysis is designed to examine the function of NIP45 as a nuclear retention factor. Fifteen minutes has been shown to be sufficient time for NFAT4 to be exported to the cytoplasm (Shibasaki, F. et al. (1996) Nature 382:370-373). All samples were then fixed and analyzed by immunofluorescence for translocation of NFAT4 as described above. The results are summarized below in Table 1. Subcellular localization of NFAT4 in the cytoplasm is indicated by a (−) and nuclear translocation of NFAT4 is indicated by (⁺).

TABLE 1
Nuclear Translocation of NFAT4

| Time | Ionomycin | Ionomycin + NIP45 |
|---|---|---|
| 0 min. | − | − |
| 2 min. | +/− | +/− |
| 4 min. | +/− | +/− |
| 8 min. | + | + |
| 15 min. | + | + |
| 15 min. + 15 min. rest | − | − |

No difference in the rate of nuclear import or export of NFAT4 was observed in the presence of NIP45, indicating that nuclear trafficking of NFAT4 in response to changes in intracellular calcium levels was not affected by the overexpression of exogenous NIP45.

### EXAMPLE 6

### Functional Activity of NIP45 in Regulating Gene Expression

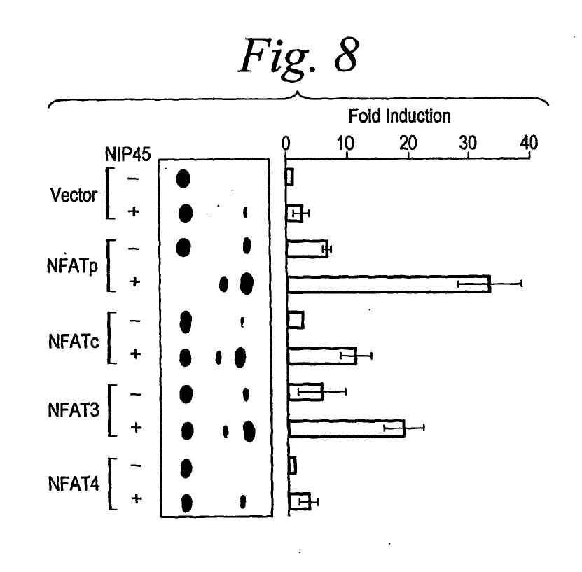

To test for a functional role of NIP45 in NFAT-driven transcription, NIP45 was expressed at high levels in HepG2 cells. HepG2 cells were chosen because they have low levels of endogenous NFAT, and ectopic expression of NFAT family member proteins has been shown to transactivate NFAT-driven transcription in this cell line in the absence of exogenous stimulation (Hoey, T. et al. (1995) Immunity 2:461-472). HepG2 cells were transfected with a 3X NFAT-CAT reporter from the IL-2 gene (Venkataraman, L. et al. (1994) Immunity 1:189-196) and control or expression plasmids for a NIP45 and NFAT family members (NFATp, NFATc, NFAT3, NFAT4). HepG2 cells were transfected by the DEAE-Dextran method as described in Hoey, T. et al. (1995) supra, and CAT assays were performed according to standard methodologies. The results are shown in FIG. 8. One representative assay for each combination is shown adjacent to a bar graph representing relative CAT activity for each group. Fold induction was calculated by normalizing the CAT activity of cells transfected with the CAT reporter and each parental expression vector to one. Values represent the relative level of CAT expression above this control transfection. All transfections were performed at least three times with one representative autoradiograph shown.

Transfection of NIP45 alone into HepG2 cells with a 3X NFAT-CAT reporter did not lead to a significant increase in CAT expression demonstrating that NIP45 cannot act on its own to transactivate an NFAT target sequence. Overexpression of NFATp alone resulted in substantial (6-fold over vector control) transactivation of the NFAT-CAT reporter, consistent with previous reports (Hoey, T. et al. (1995) supra). Cotransfection of NIP45 plus NFATp resulted in a 4-5 fold increase in CAT activity relative to transfection with NFATp alone and a 25-30 fold increase over that seen with vector alone. This increase was not observed when a mutant 3X NFAT-CAT reporter or a control MHC class II promoter reporter was used thus demonstrating its target site specificity. To confirm that the polypeptide product encoded by the NIP45 cDNA was responsible for this enhanced transactivation, a frame shift mutation was introduced in the coding region by creating a two base deletion at nucleotide 501. This alteration results in the introduction of missense mutations at amino acid 13 and termination of the polypeptide after an additional 22 residues. Assays using this NIP45Δ construct demonstrated its failure to transactivate the NFAT reporter in the presence or absence of NFATp thus confirming that the enhanced transactivation observed was due to the polypeptide expressed from NIP45 cDNA. Transactivation experiments were also performed in the B cell line M12 and the T cell clone D10 with similar although less dramatic results, which may be due to higher levels of endogenous NIP45 or NFATp in these latter cell lines. These experiments demonstrate that NIP45 substantially and specifically potentiates transcription induced by NFATp, an activity that requires interaction with NFATp.

NFAT proteins share approximately 70% identity within the RHD, raising the possibility that NIP45 could also interact with other NFAT family members. To test this, NIP45 was cotransfected as above with expression constructs encoding either NFATc, NFAT3 or NFAT4 plus the 3X NFAT-CAT reporter plasmid. The results of these experiments are also shown in FIG. 8. It has previously been demonstrated that all NFAT family members can transactivate a reporter gene containing 3 copies of an NFAT/AP1 site when overexpressed in HepG2 cells, although to different levels (Hoey, T. et al. (1995) supra). In the absence of NIP45, NFATp was the most potent transactivator of the NFAT-CAT reporter followed by NFATc and NFAT3 with only weak transactivation by NFAT4, consistent with previous data (McCaffrey, P. G. et al. (1993) Science 262:750-754). When NFATc, NFAT3 or NFAT4 were cotransfected with NIP45, NIP45 substantially potentiated both NFATc and NFAT3-driven transactivation and weakly potentiated NFAT4-mediated transactivation (FIG. 8). Cooperation with NFATc in HepG2 cells is consistent with the observation that NIP45 interacts with an NFATc RHD bait in yeast cells. Overall, NIP45 overexpression resulted in a 4-fold increase in transactivation by NFATc, a 3-fold increase in NFAT3-driven transactivation and a 2-fold increase in NFAT4-driven transcription. The ability of NIP45 to potentiate the activity of all NFAT family members is not surprising given the high degree of sequence conservation of the RHD of the NFAT family members. A sequence comparison of the NFAT RHD domains reveals a higher level of sequence identity in the amino terminal portion compared to that of the carboxyl terminus (Hoey, T. et al. (1995) supra). Thus it is likely that the NIP45/NFAT interaction site is located in the 5′ portion of the RHD.

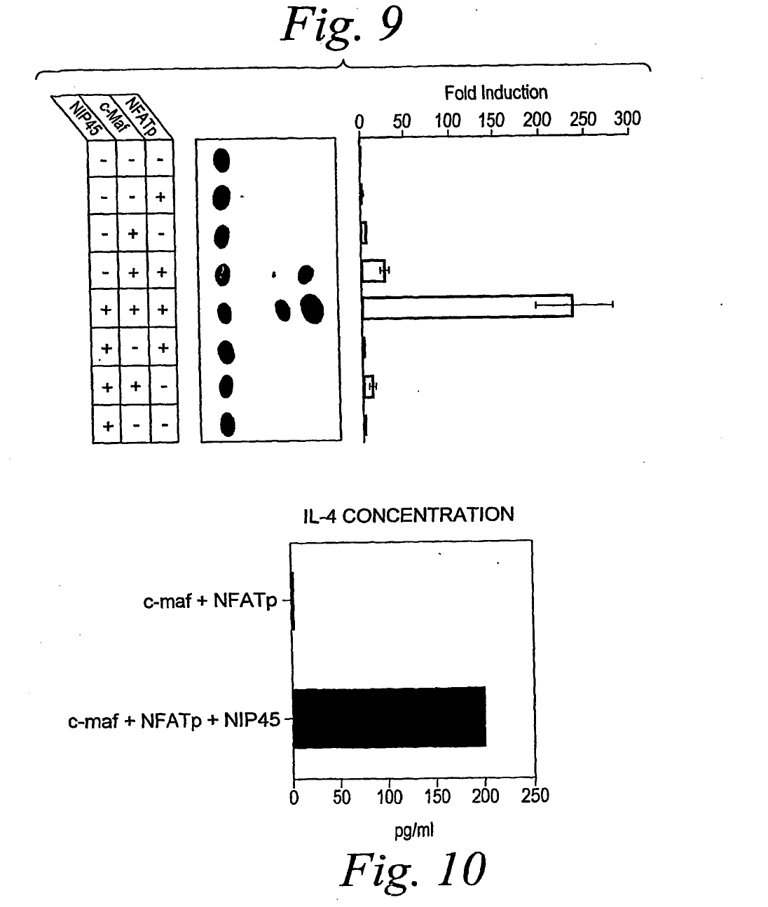

Although a reporter construct containing multiple copies of the NFAT binding site provides a sensitive method for measuring transactivation by NFAT and NIP45, it was determined if NIP45 was functional in the context of a native NFAT-dependent promoter. IL-4 expression is highly tissue specific and restricted to the Th2 subset of T cells and to mast cells. The IL-4 promoter contains multiple NFAT binding sites which have been shown to be critical for expression of IL-4 (Rooney, J. W. et al. (1995) Immunity 2:473-483). Furthermore, the proto-oncogene c-maf has been shown to direct tissue specific expression of IL-4 (U.S. Ser. No. 08/636,602). Thus, the IL-4 promoter is not active in the HepG2 cell line but can be activated by the introduction of NFATp and c-maf. In cotransfection experiments carried out as described above, HepG2 cells were transfected with an IL-4-CAT reporter construct (extending to −732 bp of the IL-4 promoter) and expression vectors or controls for NIP45, NFATp and c-Maf. The controls for NIP45 was a frame shift mutant at amino acid 13. Controls for NFATp and c-Maf were the empty expression vectors pREP4 and pMEX respectively (Ho, I. C. et al. (1996) Cell 85:973-983). The results of these experiments are shown in FIG. 9 (representative CAT assays and bar graphs are depicted as in FIG. 8). The data indicate that introduction of NIP45 together with NFATp and c-Maf results in an additional 9-fold increase in the activity of the IL-4 promoter relative to that seen for NFATp and c-Maf alone. NIP45 also increased the activity of the IL-4 promoter in the absence of transfected NFATp, an effect likely due to interaction with endogenous NFATp.

### EXAMPLE 7

### Transient Overexpression of NIP45 with NFATp and c-Maf Results in Endogenous IL-4 Production

To determine whether the combination of NIP45, NFATp and c-Maf was sufficient to induce endogenous IL-4 expression by cells that do not normally produce IL-4, M12 B lymphoma cells were transiently cotransfected with expression plasmids for NFATp and c-Maf together with NIP45 or pCI vector control. M12 cells were transiently transfected by electroporation as previously described (Ho, I. C. et al. (1996) Cell 85:973-983) by incubating 3×10⁶ cells in 0.4 ml of PBS with 5 μg of each plasmid for 10 minutes at room temperature prior to electroporation at 975 μF, 280 V. Levels of IL-4 in the supernatants harvested 72 hours later were measured by a commercially available IL-4 ELISA (Pharmingen), performed according to the manufacturer's instructions except with modification as described (Ho, I. C. et al. (1996) supra). Four independent sets of transient transfections were done and assayed for secretion of IL-4 into the culture supernatant. Results from a representative experiment from one of the four independent transfections is shown in FIG. 10. For each set of transfections, inclusion of NIP45 led to a dramatic increase in IL-4 production. Cells transfected with NIP45 produced 50-200 fold more endogenous IL-4 than cells that did not receive NIP45, in which IL-4 production was near the limit of detection.

### EXAMPLE 8

### Inhibition of Arginine Methylation Influences T Helper Cytokine Production

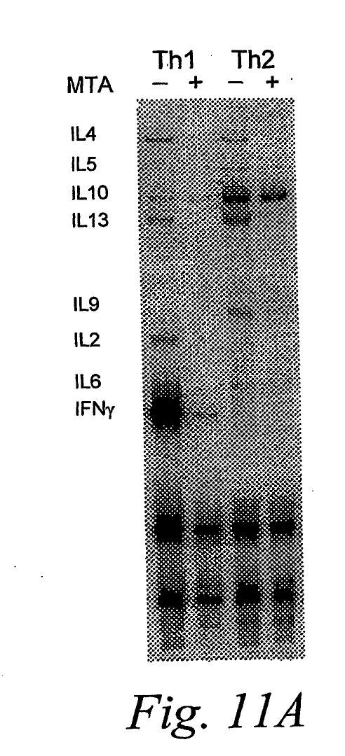

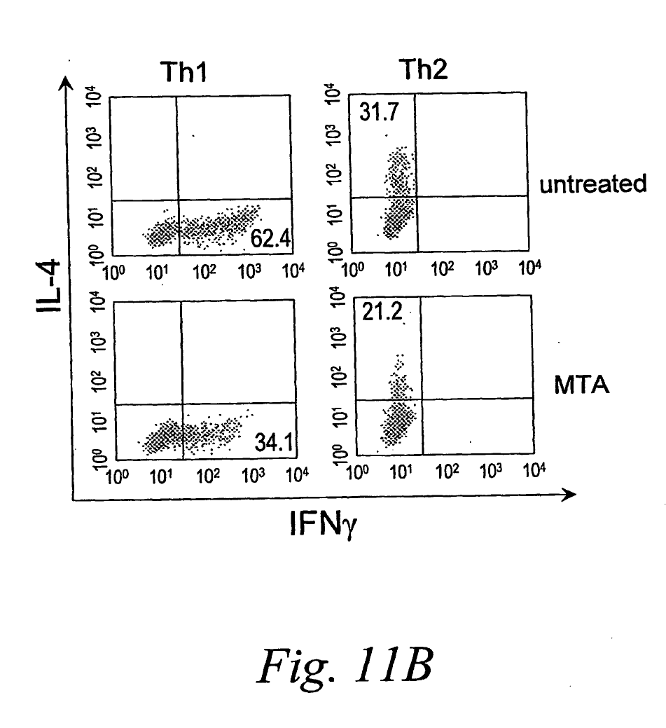

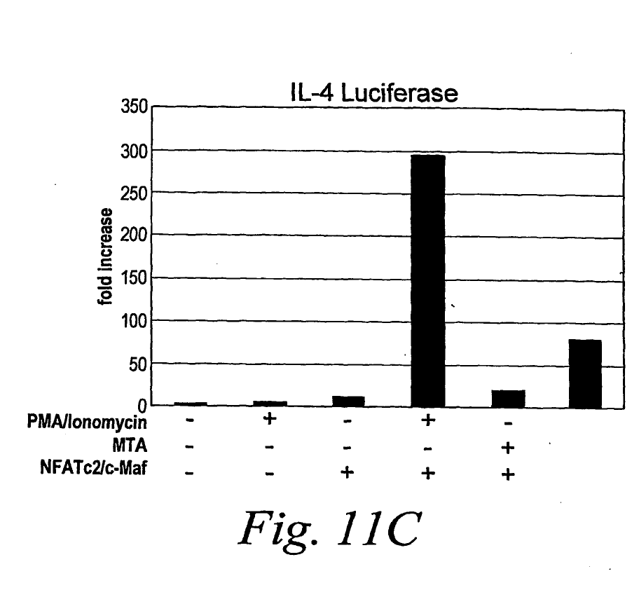

To test whether protein arginine methylation could play a role in T helper cell cytokine production, 5′-methyl-thioadenosine (MTA), a specific inhibitor of protein methyltransferases (William-Ashman, H. G., et al. (1982) Biochem Pharmacol 31:277-288) was utilized. Previous studies demonstrated that incubation of cells with MTA reduced the total amount of cellular protein methylation (Maher, P. A. (1993) J Biol Chem 268:4244-4249). DO11.10 TCR transgenic CD4⁺ lymph node cells were stimulated under Th1 or Th2 differentiating conditions for one week. Pretreatment with 1 mM MTA for 1 hour prior to a 3 hour stimulation with PMA/ionomycin led to a decrease in select cytokine mRNAs as detected by Rnase protection analysis. Specifically, decreases in IFNγ and IL-2 transcripts in Th1 cells were observed as were decreases in IL4, IL5, and IL-13 transcripts in Th2 cells, but IL-10 and IL-6 mRNA levels in Th2 cells were not affected (FIG. 11A). Because the BALB/c Th2-prone strain was used, the Th1 cultures are incompletely, polarized and still generate MTA-sensitive IL-4 transcripts (Guler, M. L., et al. (1997) J Immunol 159:1767-1774; Launois, P., et al. (1997) J Immunol 158:3317-3324). Inhibition of methyltransferase activity resulted not only in decreased levels of cytokine transcripts but also in reduction of the number of IFNγ-producing Th1 cells and IL-4 producing Th2 cells (FIG. 11B). Importantly, MTA treatment of Th1 and Th2 cells did not result in increased cell death as measured by annexin V staining (data not shown). These results suggest that PRMT directed arginine methylation regulates T helper cytokine production.

Because PRMTs could affect levels of cytokine RNA through altering promoter activation or through affecting RNA stability, the effect of MTA treatment on the activity of a Th2 selective portion of the IL4 promoter (−760 to ⁺68) was tested. This portion of the IL-4 promoter is very responsive to transactivation by the Th2 specific factor, c-Maf, in conjunction with NFAT (Szabo, S. J., et al. (1993) Mol Cell Biol 13: 4793-4805; Wenner, C. A., et al. (1997) J Immunol 158:765-773). Jurkat cells, a human T cell line were transfected with an IL-4 luciferase reporter along with NFATc2 and c-Maf expression vectors. Coexpression of NFATc2 and c-Maf greatly induced IL-4 promoter activity under PMA/Ionomycin stimulation conditions; however, pretreatment with MTA prior to PMA/ionomycin stimulation inhibited NFATc2 and c-Maf-driven IL-4 promoter transactivation (FIG. 11C), suggesting that the decrease in IL4 RNA in Th2 cells was at least partly due to inhibition of IL4 promoter activation.

### EXAMPLE 9

### PRMT is Highly Expressed in T helper Cells

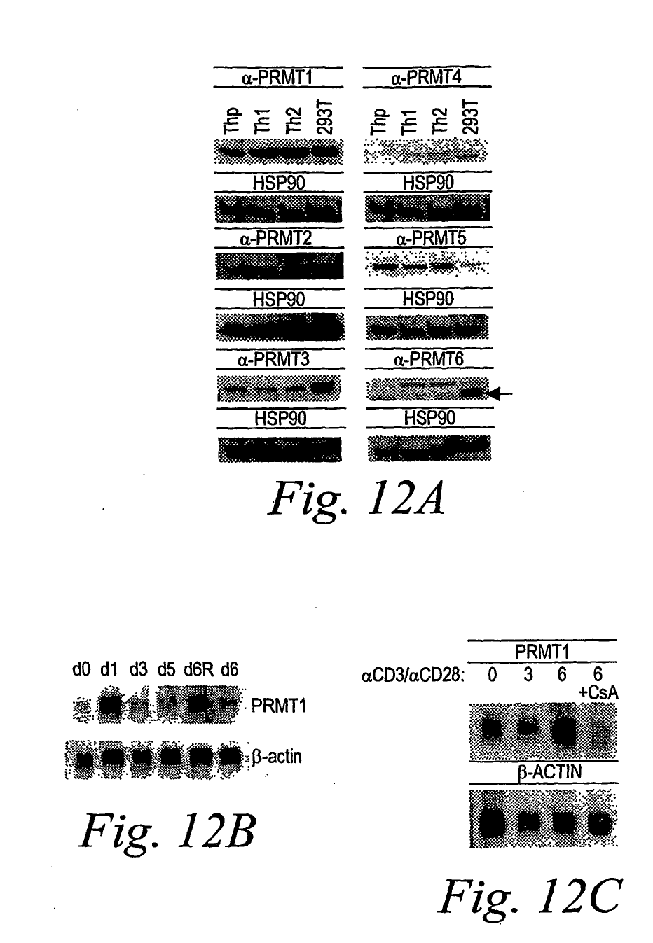

The data in Example 8 revealed an important role for arginine methylation in regulating cytokine gene expression. To determine which protein arginine methyltransferase(s) (PRMT) was the target of MTA inhibition the expression pattern of PRMTs in T helper cells as they differentiated was characterized. Cell lysates from T helper precursors (Thp) and from cells stimulated under Th1 or Th2 conditions for seven days were subjected to immunoblot analysis for PRMT, PRMT2, PRMT3, CARM1, PRMT5 and PRMT6. CARM1 and PRMT6 were expressed at low levels although CARM1 was not detected at the Thp stage; PRMT2, PRMT3, and PRMT5 were expressed at moderate levels in Thp, Th1, and Th2 cell lysates (FIG. 12A). Not only was PRMT the most highly expressed PRMT but its expression was upregulated in Th1 and Th2 cells (FIG. 12A). Induction of CARM1 expression in Th1 and Th2 cells was also observed. Because PRMT expression was both robust and regulated during Th differentiation its control was further studied. CD4 T cells were isolated from DO11.10. TCR transgenic mice and were left unstimulated or stimulated with α-CD3/α-CD28 polyclonal antibody for the indicated times. PRMT transcripts were present in unstimulated CD4⁺ T cells, but were dramatically upregulated 24 hrs after TCR stimulation (FIG. 12B). After 3 and 5 days in culture PRMT transcripts declined to levels slightly above baseline Thp expression, and when Day 5 cultures were restimulated with α-CD3, PRMT transcripts were again upregulated (sample D6R, FIG. 12B). Upregulation of PRMT transcripts was observed by 6 hours after α-CD3/α-CD28 polyclonal antibody stimulation and was cyclosporin A sensitive (FIG. 12C), indicating that PRMT1 expression was induced through TCR signaling in an NFAT-dependent manner.

These data support earlier findings that PRMT1 accounts for the majority of PRMT activity in mammalian cells (Tang, J., et al. (2000) J Biol Chem 275:7723-7730) as further evidenced by a greater than 50% reduction in the level of asymmetric dimethylarginine in deficient ES cells (Pawlak, M. R., et al. (2002) J Cell Biochem 87: 394-407). Thus, PRMT1 seemed likely to be the primary methyl transferase responsible for modulating TCR-regulated cytokine production.

### EXAMPLE 10

### PRMT Augments IFNγ and IL-4 Promoter Activity

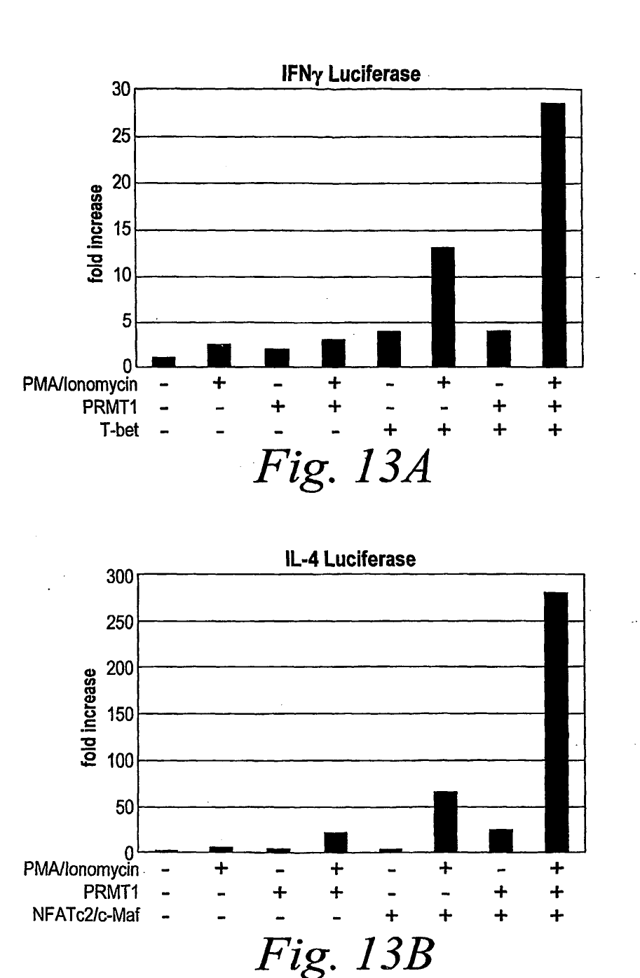

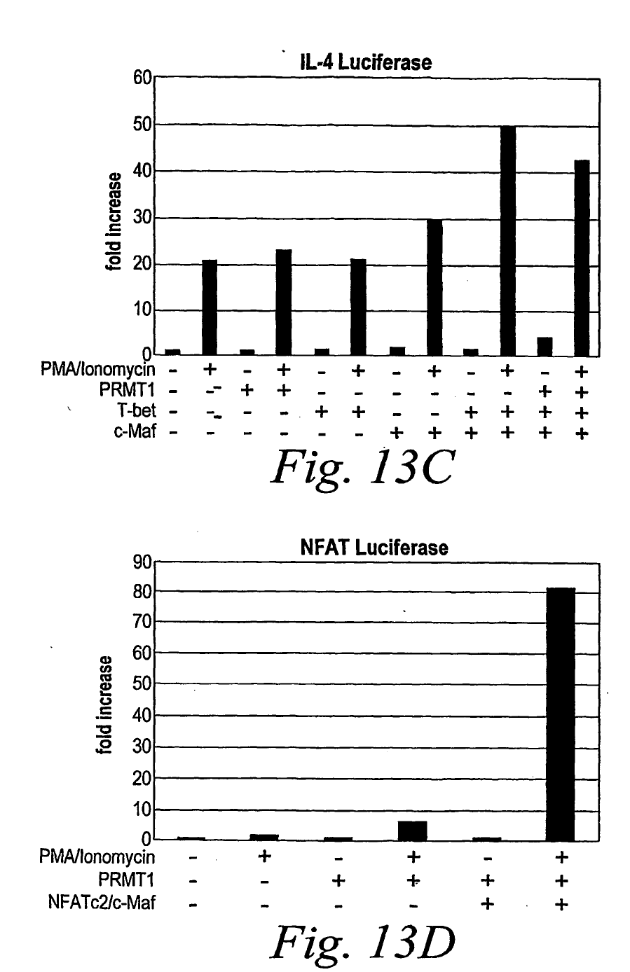

To determine whether PRMT1 could induce IFNγ and IL-4 promoter activity on its own or in conjunction with T helper transcription factors was examined. Jurkat cells were cotransfected with a 9.2 kb IFNγ luciferase reporter construct and PRMT1 alone or together with T-bet, a Th1 specific factor which regulates IFNγ expression (Szabo, S. J., et al. (2000) Cell 100:655-669). Transfection of PRMT1 alone did not transactivate the IFNγ promoter even with PMA/Ionomycin stimulation, and, as described previously, provision of T-bet induced IFNγ promoter activity under unstimulated and PMA/Ionomycin stimulation conditions (FIG. 13A). Notably, cotransfection of PRMT 1 and T-bet resulted in an enhancement of T-bet regulated IFNγ reporter, activity (FIG. 13A). Similarly, Jurkat cells transfected with PRMT1 only exhibited little induction of the IL-4 promoter over baseline even, with PMA/Ionomycin stimulation (FIG. 13B) while coexpression of NFATc2 and c-Maf greatly induced IL-4 luciferase activity (FIG. 13B). Similar to T-bet, provision of PRMT1 along with NFATc2 and c-Maf substantially augmented IL-4 promoter activity under PMA/ionomycin stimulation conditions (5-fold over NFATc2/c-Maf only) (FIG. 13B).

The IL-4 promoter contains several composite NFAT/AP-1 sites. Whether PRMT1 coactivation is mediated through NFAT or AP-1 factors was investigated as both NFAT and JunB are involved in the regulation of IL-4 promoter activity (Li, B., et al. (1999) EMBO J 18:420-432). C-Maf, also a member of the AP-1 family, synergizes with JunB to induce IL-4 promoter activity, likely as a result of cooperative DNA binding (Li, B., et al. (1999) EMBO J 18:420-432). To test whether PRMT1 augmented JunB/c-Maf transactivation of the IL-4 promoter, PRMT1, JunB, and c-Maf were cotransfected with the IL-4 luciferase reporter. Expression of JunB, or c-Maf alone with the IL-4 promoter had little effect on PMA/ionomycin stimulated reporter activity (20-30 fold compared to 20-fold vector control), however, as previously reported, under PMA/ionomycin stimulation, cotransfection of JunB and c-Maf led to a 50 fold increase compared to vector control. PRMT1 expression alone, as shown in FIG. 13B, had no effect on promoter activity or on JunB/c-Maf induced IL-4 reporter activity (FIG. 13C). In contrast, PRMT1 synergized with NFATc2 to augment NFATc2 transactivation of an NFAT reporter element by approximately 10-fold (FIG. 13D). Thus, PRMT1-induced transcriptional enhancement occurs with both the IFNγ and IL-4 promoters and at least in the case of IL-4 promoter induction, is mediated through the NFAT transcription factor.

### EXAMPLE 11

### NIP45 is a Potential PRMT Target in T helper Cells

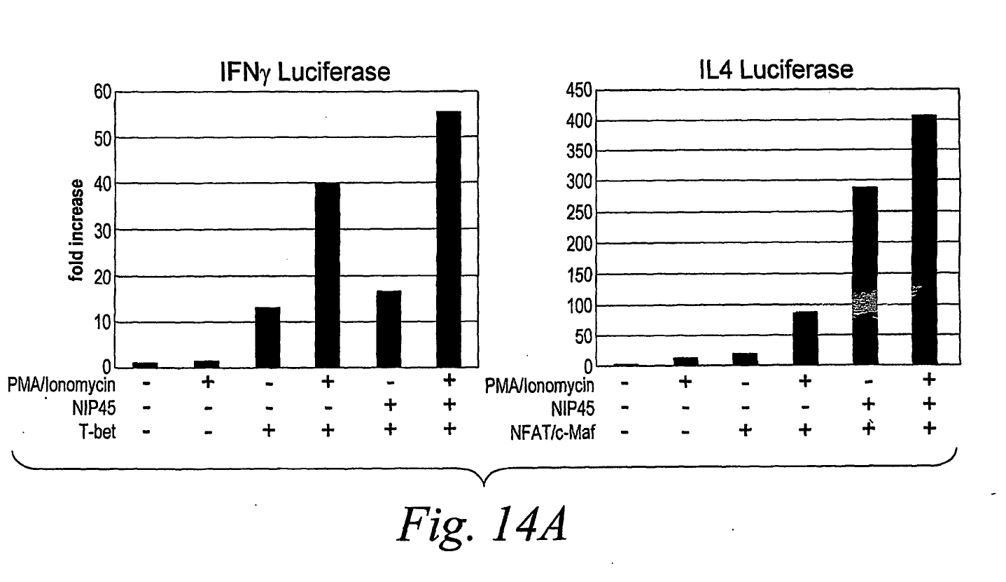

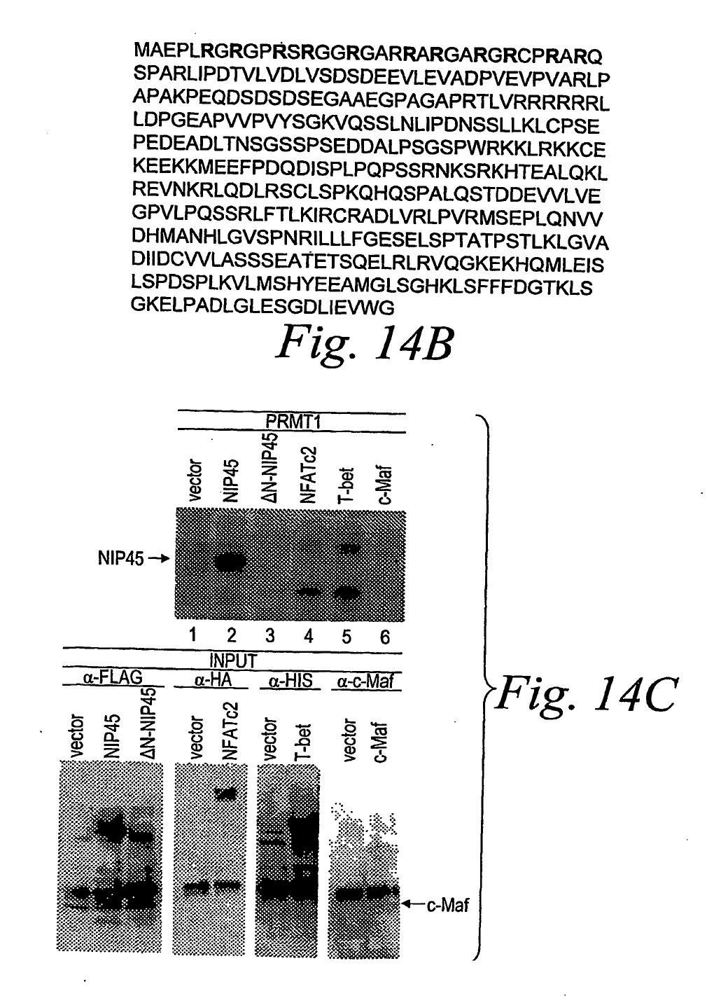

Potential targets of PRMT1 methyltransferase activity were identified. The effects of PRMT1 on the IFNγ and IL4 promoter were reminiscent of an NFAT-interacting factor previously isolated (Hodge, M. R et al. (1996) Immunity 4: 1-20). NFAT interacting protein (NIP) 45 kd is a cofactor in NFATc2/c-Maf driven IL-4 promoter activity and endogenous IL-4 expression (FIG. 14A and Hodge, M. R et al. (1996) Immunity 4:1-20), and has subsequently been observed to have a similar synergy between NIP45 and T-bet in transactivating an IFNγ reporter construct (FIG. 14A). Furthermore, the ammo-terminus (a.a. 6-32) of NIP45 contains several RXR and RG motifs which have been shown to be favorable for methylation by Type I PRMTs (McBride, A. E., and Silver, P. A. (2001) Cell 106:5-8) (FIG. 14B). Within the amino-terminus, NIP45 contains eleven arginine residues which are potentially methylated. Whether PRMT1 could methylate NIP45, NFATc2, T-bet or c-Maf was tested. Transfected cell lysates were harvested and immunoprecipitated with indicated antibodies to isolate NIP45, NFATc2, T-bet, or c-Maf. Immunoprecipitates were subjected to an in vitro methylation assay using recombinant PRMT1. PRMT1 methylated immunoprecipitated NIP45 but not ΔN-NIP45 in which amino acids 1-32 are deleted (FIG. 14C lanes 2 and 3 upper panel). Additionally, PRMT1 did not methylate NFATc2, c-Maf, or T-bet immunoprecipitates, indicating that of these factors NIP45 is the only potential target of PRMT1 (FIG. 14C lanes 2, 4, 5, 6 top panel).

### EXAMPLE 12

### NIP45 is Methylated by PRMT

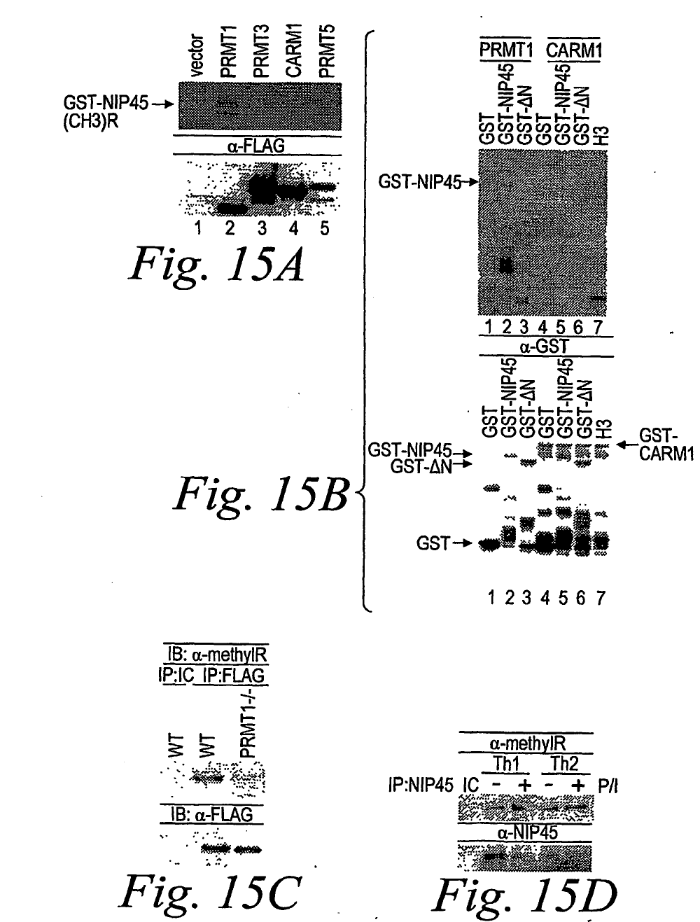

Although it was shown that PRMT1 can methylate NIP45, this did not rule out the possibility that other PRMTs can methylate NIP45 as well. In vitro methylation assays were utilized to compare the ability of affinity purified PRMT1, PRMT3, CARM1, and PRMT5 to methylate a bacterially expressed GST-NIP45 fusion protein. In this assay, only PRMT1 was capable of methylating NIP45 (FIG. 15A lane 2 upper panel). As PRMT1 is known to form homodimers, the possibility exists that it may form heterodimers with other PRMTs as well; thus the methylation of NIP45 observed in immunoprecipitates from cell lysates could be due to the methyltransferase activity of another PRMT that is recruited by PRMT1. Therefore whether PRMT1 could directly methylate NIP45 using recombinant proteins was tested. As shown in FIG. 15B, recombinant PRMT1 was able to methylate bacterially expressed GST-NIP45 (lane 2) whereas CARM1 did not (lane 5), although CARM1 did methylate histone H3 (lane 7) as described previously (chen 1999). These data suggest that PRMT1 methylation of NIP45 is direct and specific. To prove that the amino terminus of NIP45 was the target for methylation by PRMT1, a GST-ΔN-NIP45 fusion protein in which amino acids 1-32 were deleted was created. Elimination of the amino terminus resulted in a loss of PRMT1 induced methylation (FIG. 15B lane 3). Others have shown that methylation of proteins in vitro by PRMT1 correlates well with their methylation by PRMT1 in vivo (Pawlak, M. R., et al. (2002) J Cell Biochem 87:394-407). To test this notion directly, wildtype and PRMT1 deficient ES cells were transfected with NIP45 and Western blot analysis was performed using an antibody specific for asymmetrically methylated arginines within RG repeats similar to those found in the amino-terminus of NIP45. Methylation of NIP45 was greatly reduced in PRMT1 deficient ES cells (FIG. 15C upper panel). Next, in order to determine whether NIP45 was methylated in T helper cells, endogenous NIP45 was precipitated from lysates generated from cells grown under Th1 or Th2 conditions for seven days and immunoprecipitates probed with the anti-methylated arginine antibody. NIP45 was methylated in both Th1 and Th2 skewed lysates and the methylation status of NIP45 did not change after PMA/Ionomycin stimulation for 60 minutes (FIG. 15D upper panel). Therefore, NIP45 is a substrate of PRMT1, the amino terminus of NIP45 is necessary for methylation by PRMT and NIP45 is methylated in vivo.

### EXAMPLE 13

### Association Between NIP45 and PRMT

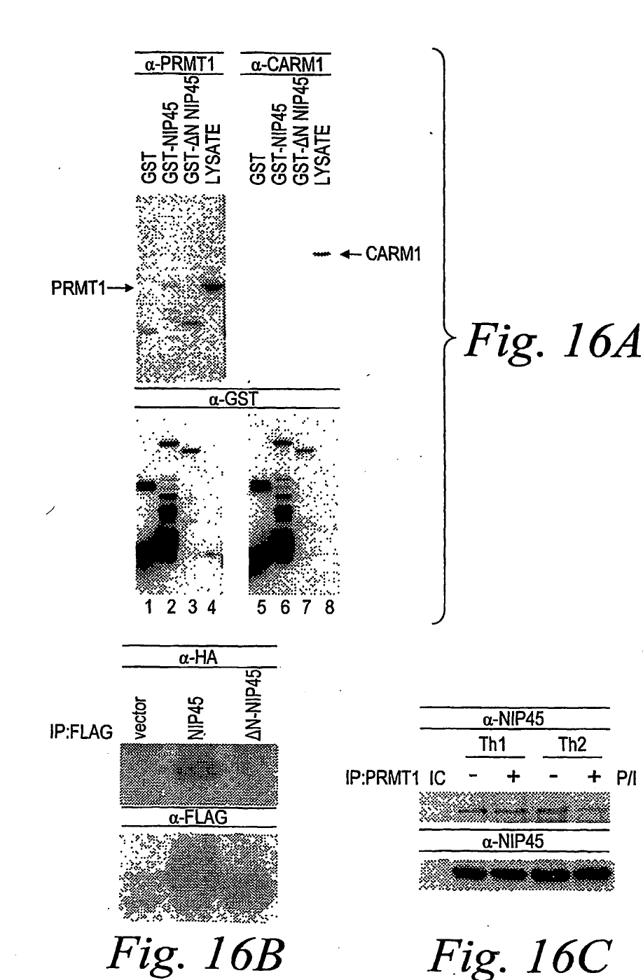

Because PRMT1 methylates NIP45, their interaction was examined more closely. To determine specificity of interaction GST-NIP45 and GST-ΔN-NIP45 fusion proteins were used in pulldown assays of Jurkat cell lysates. Similar to the in vitro methylation results, PRMT1 but not CARM1 associated with full length NIP45 (FIG. 16A lanes 2 and 6 upper panel). Furthermore, the interaction between NIP45 and PRMT1 was dependent on the amino-terminus of NIP45 (FIG. 16A lane 3). To parallel the pulldown assays, 293T cells were transfected with HA-PRMT1, FLAG-NIP45, and FLAG-ΔN NIP45 mutant. PRMT1 coimmunoprecipitated with full length NIP45 but not with the ammo-terminal deletion mutant (FIG. 16B), indicating that not only is this region a potential site of PRMT1 methylation but that it is also necessary for a physical NIP45/PRMT1 interaction. Thus, the selectivity and amino-terminus dependence of the NIP45 interaction with PRMT1 in vivo mirrors the specificity observed in the in vitro methylation assays.

In order to evaluate the physiologic significance of the PRMT1 and NIP45 interaction, coimmunoprecipitation assays were performed using lysates from primary T helper cells differentiated for 7 days under Th1 or Th2 conditions unstimulated, or stimulated for 60 minutes with PMA/ionomycin. Lysates were immunoprecipitated using an anti-PRMT1 antibody as well as the appropriate isotype control and immunoblotted using anti-NIP45 mononclonal antibodies to detect the associated NIP45 protein (FIG. 16C upper panel). Blots were reprobed with anti-PRMT1 antibody to determine equal protein loading (FIG. 16C lower panel). These experiments revealed that endogenous PRMT1 and NIP45 proteins associate with each other within primary T helper cells, and that this association does not change under conditions mimicking TCR stimulation.

### EXAMPLE 14

### The Amino-Terminus of NIP45 is Important for its Function

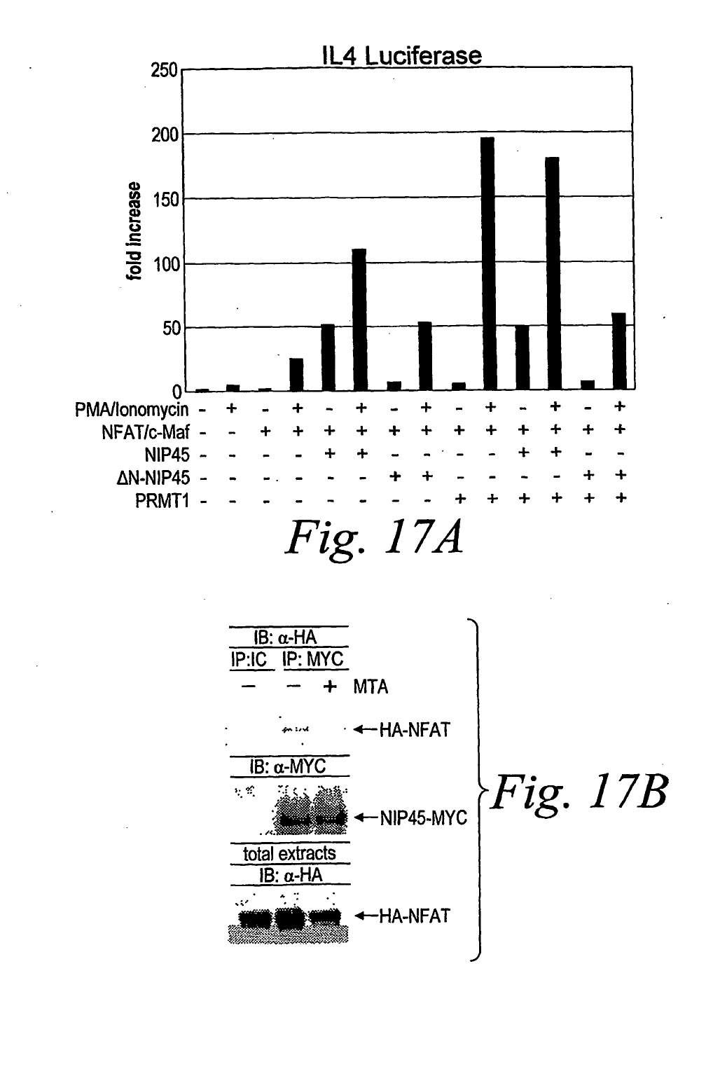

Since the amino-terminus of NIP45 is methylated and important for interaction with PRMT1, whether the arginine methylation domain of NIP45 within the ammo-terminus was necessary for NIP45 function as an NFAT cofactor was investigated. To determine the association ability of recombinant ΔN-NIP45 and full-length NIP45 with NFAT, pull-down assays with HA-NFATc2-transfected Jurkat cell lysates were performed. The ΔN-NIP45 mutant had reduced NFATc2 binding ability, demonstrating that the amino terminus of NIP45 is important for int interaction with NFAT. To assess the co-factor function of ΔN-NIP45, Jurkat cells were transfected with different combinations of PRMT1, NFATc2, c-Maf, NIP45, and ΔN-NIP45 to test their effects on IL-4 promoter activity. As above, PRMT1 greatly enhanced NFATc2 and c-maf promoter transactivation as well as IL-4 promoter activity, but did not transactivate the IL-4 promoter on its own but instead synergized with NF ATc2 and c-Maf (FIG. 17A). Deletion of the NIP45 amino terminus reduced the transactivation ability of NIP45 under unstimulated and PMA/ionomycin stimulation conditions (FIG. 17A). To ensure that deletion of the NIP45 amino terminus did not alter its subcellular localization, ΔN-NIP45 was expressed fused to green fluorescent protein in mouse embryonic fibroblasts and found nuclear localization was still intact. Therefore, the impaired function of ΔN-NIP45 was not due to aberrant cellular localization. When NIP45 and PRMT1 were cotransfected with NFATc2 and c-Maf, reporter activity was not stimulated beyond that seen with just PRMT1, NFATc2, and c-Maf; however, when ΔN-NIP45 was expressed in conjunction with PRMT1, NFATc2, and c-Maf, the dramatic augmentation of IL-4 promoter activity by PRMT1 was lost (FIG. 7A). These data suggest that the ammo-terminus of NIP45 is necessary for potent NIP45-induced augmentation of NFAT transactivation and is responsible for PRMT1 coactivation of the IL-4 promoter. Indeed, NFATc2, NIP45, and PRMT1 form a ternary complex as evidenced by the fact that PRMT1 and NFATc2 association is substantially promoted by the presence of full-length NIP45, but much less so by the ΔN-NIP45. These data show that the amino terminus of NIP45 serves a dual function in supporting NFAT interaction and recruiting PRMT1 to the NFAT transcription-activating complex.

The function of arginine methylation in regulating NIP45 activity was investigated. Arginine methylation has previously been shown to regulate subcellular localization and protein-protein interactions. Because ΔN-NIP45 is still localized to the nucleus, it was unlikely that arginine methylation of the ammo-terminus of NIP45 was necessary for its presence in the nucleus. Since NIP45 binds to NFATc2 and augments NFATc2 driven IL-4 promoter activity (Hodge, M. R et al. (1996) Immunity 4:1-20), the effects of MTA treatment on the interaction between NIP45 and NFATc2 was tested. cMyc-tagged NIP45 and HA-tagged NFATc2 were coexpressed in 293T cells which were left untreated or treated with 1 mM MTA for 60 minutes. Inhibition of arginine methylation reduced the association between NIP45 and NFATc2 (FIG. 7B), demonstrating that the function of arginine methylation of NIP45 is to modulate this interaction.

### EXAMPLE 15

### NIP45 Deficient Mice have Defects in Th Cell Cytokine Production as Well as Non-T Cell Cytokine Production

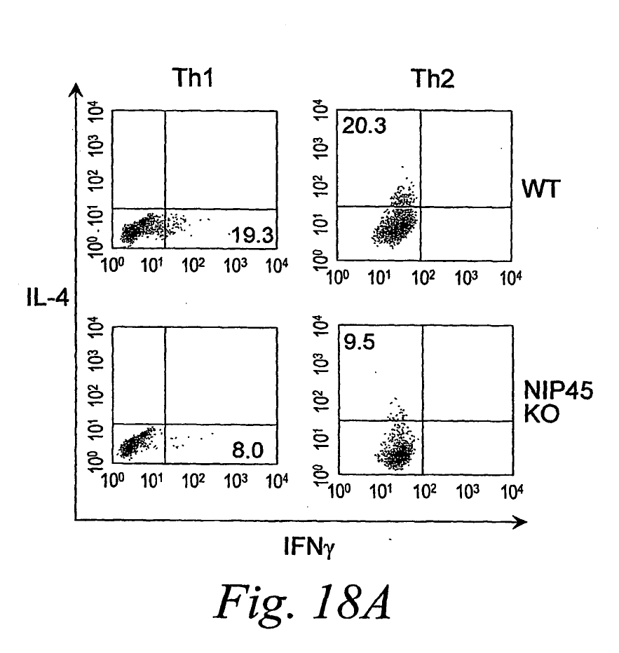

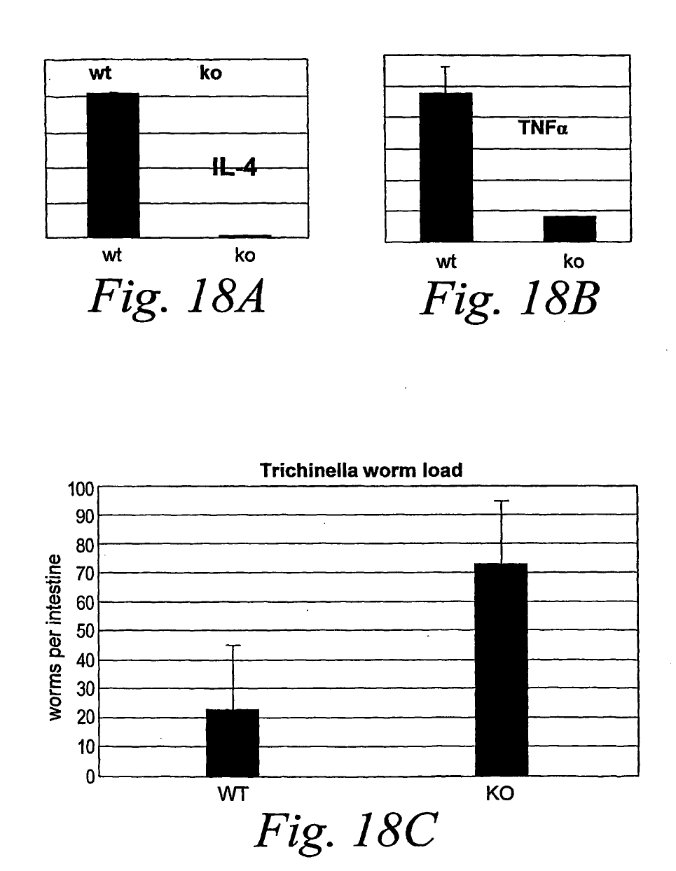

NIP45^{-/-} mice were generated by standard techniques, and the absence of NIP45 protein in lymphoid cells confirmed ablation of NIP45. There were normal numbers of T and B cells and phenotypic analysis and receptor-induced proliferation of these populations revealed no obvious defects. The ability of NIP45^{-/-} Th cells to produce cytokines was tested. FIG. 18 (A) depicts the results of intracellular cytokine (ICC) FACS of naive CD4⁺ T cells isolated by FACS sorting for CD4⁺ Mel14⁺ cells and stimulated under Th1 (IL-12 and anti-IL4) and Th2 (IL-4, and anti-IFNγ) conditions with platebound antiCD3/antiCD28. Cells were split out with IL2 on day 3 and analyzed on day 7 after stimulation with PMA/ionomycin by ICC. FIGS. 18 (B-D) show that NIP45 deficient mice have profound defects in mast cell cytokine production and function.

BMMC from NIP45^{+/+} (wt) and ^{-/-} (ko) mice were cultured for 3 weeks in IL3 and 1×10⁶ cells/ml were stimulated with PMA/ionomycin. Supernatants were collected 24 hrs later and assayed for cytokine production (A and B). NIP45 wt and ko mice were infected with T. spiralis larvae. Twelve days later mice were sacrificed and intestinal worm burden was determined (C). Wild-type animals had an average of slightly greater than 20 worms/intestine, while knock out animals had slightly greater than 70 worms/intestine.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of identifying a compound that modulates cytokine production, comprising:
a) providing an indicator composition comprising a type I polypeptide arginine methyltransferase (PRMT1) polypeptide;
b) contacting the indicator composition with a plurality of test compounds;
c) selecting from the library of test compounds a compound of interest that modulates an activity of PRMT1;
to thereby identify a compound that modulates cytokine production.

2. A method of identifying a compound that modulates T cell receptor-mediated signaling, comprising:
a) providing an indicator composition comprising a type I polypeptide arginine methyltransferase (PRMT1) polypeptide;
b) contacting the indicator composition with a plurality of test compounds;
c) selecting from the library of test compounds a compound of interest that modulates an activity of PRMT1;
to thereby identify a compound that modulates T cell receptor-mediated signaling.

3. The method of claim 1, wherein the activity of PRMT1 is a NIP45-related activity.

4. A method of identifying a compound that modulates cytokine production, comprising:
a) providing an indicator composition comprising the upstream regulatory regions controlling expression of a type I polypeptide arginine methyltransferase (PRMT1) polypeptide operably linked to a reporter gene;
b) contacting the indicator composition with a plurality of test compounds;
c) selecting from the library of test compounds a compound of interest that modulates the expression of the reporter gene;
to thereby identify a compound that modulates cytokine production.

5. The method of claim 4, further comprising determining the effect of the compound of interest on a NIP45-related activity of PRMT1.

6. The method of claim 1, wherein the indicator composition is a cell that expresses the PRMT1 polypeptide.

7. The method of claim 6, wherein the cell has been engineered to
express the PRMT1 polypeptide by introducing into the cell an expression vector encoding the PRMT1 polypeptide.

8. The method of claim 1, wherein the indicator composition is a cell free composition.

9. The method of claim 1, wherein the step of determining the effect of the compound of interest on an activity of PRMT1 comprises measuring the ability of the PRMT1 to methylate one or more arginine residues of a target polypeptide.

10. The method of claim 9, wherein the target polypeptide is NIP45.

11. The method of claim 6, wherein the cell further comprises a NIP45 polypeptide.

12. The method of claim 6, wherein the cell further comprises an NFAT polypeptide.

13. The method of claim 3, wherein the step of determining the effect of the compound of interest on a NIP45-related activity of PRMT1 comprises measuring cytokine production or cytokine gene transcription.

14. The method of claim 13, wherein the cytokine is IFN-γ or IL-4.

15. A method for identifying a compound that modulates cytokine production in a non-T cell, comprising;
a) providing non-T cell comprising a NIP45 molecule;
b) contacting the non-T cell with a plurality of test compounds; and
c) selecting from the library of test compounds a compound of interest that modulates the activity of NIP45;
to thereby identify a compound that modulates cytokine production in a non-T cell.

16. The method of claim 15, wherein the cell further comprises PRMT1.

17. The method of claim 15, wherein the cell further comprises NFAT.

18. The method of claim 15, wherein the activity of NIP45 is selected from the group consisting of: binding to NFAT, binding to PRMT1, and activation of gene transcription.

19. The method of claim 18, wherein the gene is selected from the group consisting of: IL-4, IFN-γ, Egr2, Egr3, c-Rel, and p65.

20. A method for identifying a compound that modulates gene expression comprising:
a) contacting an indicator composition comprising a first polypeptide comprising amino acids 1-32 of NIP45 and a second polypeptide which is a PRMT1 polypeptide with a plurality of test compounds;
b) detecting an activity of the first polypeptide or a NIP45-related activity of the second polypeptide in the presence and absence of a test compound, and
c) selecting a compound of interest that modulates an activity of the first or second polypeptide;
to thereby identify a compound that modulates gene expression.

21. The method of claim 20, wherein the indicator composition is a cell.

22. The method of claim 20, wherein the cell further comprises an NFAT polypeptide and the activity of the first polypeptide is detected by measuring the binding of the first polypeptide to the NFAT polypeptide.

23. The method of claim 22, wherein the NFAT polypeptide is selected from the group consisting of: NFATc1, NFATc2, and NFATc3.

24. The method of claim 20, wherein the activity of the first polypeptide is detected by measuring transcription from a promoter.

25. The method of claim 24, wherein the promoter is an IL-4 or IFN-γ promoter.

26. The method of claim 24, wherein the promoter is selected from the group consisting of: the Egr2, Egr3, c-Rel, and p65 promoter.

27. The method of claim 20, wherein the indicator composition is present in a cell free system.

28. The method of claim 20, wherein the NIP45-related activity of the second polypeptide is detected by measuring the methylation of one or more arginine residues of NIP45.

29. The method of claim 20, wherein the NIP45-related activity of the second polypeptide is detected by measuring the interaction between the second polypeptide and the first polypeptide.

30. The method of claim 20, wherein the test compounds are present in a library of small molecules.

31. The method of claim 28, wherein the test compound decreases the degree of arginine methylation of NIP45 as compared to the degree of arginine methylation of NIP45 in the absence of the test compound, and the test compound is identified as an agent that reduces cytokine production.

32. The method of claim 28, wherein the test compound increases the degree of arginine methylation of NIP45 as compared to the degree of arginine methylation of NIP45 in the absence of the test compound, and the test compound is identified as an agent that increases cytokine production.

33. A method for identifying a compound that modulates an interaction between NIP45 and a PRMT polypeptide, comprising:
a) contacting an indicator composition comprising a polypeptide comprising amino acids 1-32 of NIP45 and a PRMT polypeptide with a plurality of test compounds;
b) detecting a readout of the interaction between the NIP45 and PRMT polypeptides in the presence and absence of a test compound, and
c) selecting a compound of interest that modulates the interaction between the NIP45 and PRMT polypeptides;
to thereby identify a compound that modulates an interaction between NIP45 and PRMT polypeptide.

34. The method of claim 33, wherein the indicator composition is a cell based composition.

35. The method of claim 33, wherein the indicator composition is a cell free composition.

36. The method of claim 33 wherein the readout of the interaction between the NIP45 and PRMT1 polypeptides is the binding of NIP45 to PRMT1 or the methylation of one or more arginine residues of NIP45.

37. The method of claim 33, wherein the readout of the interaction between the first and second polypeptides is modulation of gene transcription.

38. The method of claim 37, wherein the gene is selected from the group consisting of IL-4 and IFN-γ.

39. The method of claim 33, wherein said test compounds are present in a library of small molecules.

40. The method of claim 33, wherein the test compound decreases the interaction between NIP45 and PRMT1 as compared to the interaction between NIP45 and PRMT1 in the absence of the test compound, and the test compound is identified as an agent that reduces interaction between NIP45 and PRMT1.

41. The method of claim 33, wherein the test compound increases the interaction between NIP45 and PRMT1 as compared to the interaction between NIP45 and PRMT1 in the absence of the test compound, and the test compound is identified as an agent that increases interaction between NIP45 and PRMT1.

42. A method for identifying a compound that modulates cytokine production in a cell, comprising;
a) providing a cell containing one or more constructs which comprise: a cytokine promoter operably linked to a reporter gene, a nucleotide sequence encoding PRMT1, and a nucleotide sequence encoding at least one activator of cytokine gene transcription;
b) stimulating the cell with an activating signal;
c) contacting the cell with a plurality of test compounds;
d) measuring the expression or activity of the reporter gene; and
e) selecting a compound of interest that modulates the expression or activity of the reporter gene,
to thereby identify a compound that modulates cytokine production in a cell.

43. The method of claim 42, wherein the cytokine promoter is an IFNγ promoter.

44. The method of claim 42, wherein the activator of cytokine gene transcription is T-bet.

45. The method of claim 42, wherein the cytokine promoter is an IL-4 promoter.

46. The method of claim 42, wherein the activator of cytokine gene transcription is selected from the group consisting of NFATc2 and NIP45.

47. The method of claim 42, wherein the cell further comprises a construct comprising a nucleotide sequence encoding c-maf.

48.-66. (canceled)
